(19) United States
(12) Patent Application Publication
(10) Pub. No.: US 2022/0133701 A1
(43) Pub. Date: May 5, 2022

(54) METHODS OF TREATING PAIN

(71) Applicant: Yeda Research and Development Co. Ltd., Rehovot (IL)

(72) Inventors: Michael FAINZILBER, Rehovot (IL); Letizia MARVALDI, Rehovot (IL)

(73) Assignee: Yeda Research and Development Co. Ltd., Rehovot (IL)

(21) Appl. No.: 17/576,954

(22) Filed: Jan. 16, 2022

Related U.S. Application Data
(63) Continuation of application No. PCT/IL2020/050801, filed on Jul. 16, 2020.

(30) Foreign Application Priority Data
Jul. 16, 2019 (IL) 268111

(51) Int. Cl. A61K 31/437; A61K 31/496; A61P 25/04

(52) U.S. Cl. CPC A61K 31/437; A61P 25/04; A61K 31/496

(57) ABSTRACT

Methods of treating pain are provided. Accordingly, there is provided a method of treating nociceptive or neuropathic pain in a subject in need thereof, the method comprising administering to the subject a therapeutically effective amount of an agent which binds importin alpha3 or a polynucleotide encoding same and inhibits expression and/or activity of said importin α3.

### RELATED APPLICATIONS

This application is a Continuation of PCT Patent Application No. PCT/IL2020/050801 having International filing date of Jul. 16, 2020, which claims the benefit of priority of Israel Patent Application No. 268111 filed on Jul. 16, 2019. The contents of the above applications are all incorporated by reference as if fully set forth herein in their entirety.

### Sequence Listing Statement

The ASCII file, entitled 90387SequenceListing.txt, created Jan. 16, 2022, comprising 5,447 bytes, submitted concurrently with the filing of this application is incorporated herein by reference.

### FIELD AND BACKGROUND OF THE INVENTION

The present invention, in some embodiments thereof, relates to methods of treating pain.

Nociceptive pain is part of a rapid warning relay instructing the motor neurons of the central nervous system to minimize a detected physical harm. It is mediated by nociceptors, on A-δ and C fibers. These nociceptors are free nerve endings that terminate just below the skin, in tendons, joints, and in body organs. They serve to detect cutaneous pain, somatic pain and visceral pain.

Neuropathic pain is produced by dysfunction of or damage to the neurons in the peripheral and central nervous systems and involves sensitization of these systems. In peripheral sensitization, there is an increase in the stimulation of peripheral nociceptors that amplifies pain signals to the central nervous system. In central sensitization, neurons that originate in the dorsal horn of the spinal cord become hyperstimulated, increasing pain signals to the brain and thereby increasing pain sensation. It is most commonly associated with chronic allodynia and hyperalgesia.

Inflammatory pain is associated with tissue damage and the resulting inflammatory process. It is adaptive in that it elicits physiologic responses that promote healing.

Narcotic analgesic substances, such as opioids and their derivatives, are the most commonly used class of anti-pain drugs. Their long term use has been limited due to their negative side effects such as constipation, sedation, respiratory depression, and principally tolerance and physical dependence, which develop rapidly after administration. The vast majority of current targets for drug development in the pain field are ion channels and neurotransmitter receptors, localized at the plasma membrane and the synapse.

Importins are a group of proteins that transport protein molecules into the nucleus by binding to specific recognition sequences, called nuclear localization sequences (NLS). Importins are expressed in all neuronal compartments, including axons, dendrites and synapses; and their dependent transport mechanisms link synapse to nucleus in a diversity of physiological contexts. Importin has two subunits, importin α and importin β, wherein members of the importin-β subfamily can bind cargo proteins and transport them by themselves, or can form heterodimers with importin-α subunits that bind NLS cargos. There are 6-7 importin α family members in any given mammal and individual cell types express different subsets of this ensemble, often in a tightly regulated manner [Pumroy, R. A. & Cingolani, G. Biochem J 466, 13-28 (2015); and Yasuhara, N. et al. Dev Cell 26, 123-135 (2013)]. Injury in peripheral neurons or activity in central neurons can activate importin-dependent transport mechanisms in axons or dendrites to link both pre- and postsynaptic compartments to soma and nucleus [Lim, A. F et al. Neurobiol Learn Mem 138, 78-84 (2017); and Rishal, I. & Fainzilber, M. Nat Rev Neurosci 15, 32-42 (2014)]. Assigning specific roles for individuals in the importin α family in brain functions is challenging due to functional redundancies in cargo binding and compensatory expression regulation of different family members [e.g. Ushijima, R. et al. Biochem Biophys Res Commun 330, 880-886 (2005); and Shmidt, T. et al. Nat Cell Biol 9, 1337-1338; author reply 1339 (2007)].

Additional background art includes International Patent Application Publication No: WO2011112732; and US Patent Application Publication No: US20170151339.

### SUMMARY OF THE INVENTION

According to an aspect of some embodiments of the present invention there is provided a method of treating nociceptive or neuropathic pain in a subject in need thereof, the method comprising administering to the subject a therapeutically effective amount of an agent which binds importin α3 or a polynucleotide encoding same and inhibits expression and/or activity of the importin α3, thereby treating the nociceptive or neuropathic pain in the subject.

According to an aspect of some embodiments of the present invention there is provided an agent which binds importin α3 or a polynucleotide encoding same and inhibits expression and/or activity of the importin α3 for use in treating nociceptive or neuropathic pain in a subject in need thereof.

According to some embodiments of the invention, the activity comprises C-Fos nuclear transport.

According to some embodiments of the invention, the agent binds an importin α3-C-Fos complex, interferes with formation of the importin α3-C-Fos complex or disintegrates the importin α3-C-Fos complex.

According to some embodiments of the invention, the agent is a small molecule.

According to some embodiments of the invention, the agent is a RNA silencing agent.

According to some embodiments of the invention, the agent is a inhibitory peptide.

According to some embodiments of the invention, the peptide comprises a portion of C-Fos comprising an amino acid sequence of a nuclear localization sequence (NLS) of C-Fos.

According to some embodiments of the invention, the peptide comprises a portion of C-Jun comprising an amino acid sequence of a nuclear localization sequence (NLS) C-Jun.

According to an aspect of some embodiments of the present invention there is provided a method of treating pain in a subject in need thereof, the method comprising administering to the subject a therapeutically effective amount of an agent capable of inhibiting expression and/or activity of a target selected from the targets listed in Table 1.

According to an aspect of some embodiments of the present invention there is provided an agent capable of inhibiting expression and/or activity of a target selected from the targets listed in Table 1 for use in treating pain in a subject in need thereof.

According to some embodiments of the invention, the target is Syngap1 or RTL1.

According to an aspect of some embodiments of the present invention there is provided a method of treating pain in a subject in need thereof, the method comprising administering to the subject a therapeutically effective amount of an agent capable of enhancing expression and/or activity of a target selected from the targets listed in Table 2.

According to an aspect of some embodiments of the present invention there is provided an agent capable of enhancing expression and/or activity of a target selected from the targets listed in Table 2 for use in treating pain in a subject in need thereof.

According to some embodiments of the invention, the agent binds the target or a polynucleotide encoding same.

According to an aspect of some embodiments of the present invention there is provided a method of treating pain in a subject in need thereof, the method comprising administering to the subject a therapeutically effective amount of a compound selected from the compounds listed in Table 3, thereby treating pain in the subject.

According to an aspect of some embodiments of the present invention there is provided a compound selected from the compounds listed in Table 3 for use in treating pain in a subject in need thereof.

According to some embodiments of the invention, the compound is sulmazole or sulfamethizole.

According to some embodiments of the invention, the compound is selected from the group consisting of sulmazole, sulfamethizole, ajmaline, pramocaine, prasterone, MK-886, diphenylpyraline, vitexin, ciclacillin, sulfamidine, ceftazidime and profenamine.

According to some embodiments of the invention, the compound is selected from the group consisting of sulmazole, sulfamethizole, pramocaine, prasterone, MK-886, diphenylpyraline, vitexin, ciclacillin, sulfamidine, ceftazidime and profenamine.

According to some embodiments of the invention, the pain is nociceptive or neuropathic pain.

According to some embodiments of the invention, the pain is acute pain.

According to some embodiments of the invention, the pain is chronic pain.

According to some embodiments of the invention, the neuropathic pain is peripheral neuropathic pain.

According to some embodiments of the invention, the neuropathic pain is central neuropathic pain.

According to some embodiments of the invention, the pain is a peripheral denervation neuropathic pain.

According to some embodiments of the invention, the pain is an acute thermal nociceptive pain or acute mechanical nociceptive pain.

According to some embodiments of the invention, the pain is an acute chemically-induced pain.

According to some embodiments of the invention, the pain is not associated with vascular inflammation.

According to an aspect of some embodiments of the present invention there is provided a method of identifying a compound for treating pain, the method comprising determining a transcriptional signature of a neuronal cell following treatment with a test compound and comparing the transcriptional signature of the neuronal cell following the treatment to a transcriptional signature of an importin alpha3 deficient neuronal cell, wherein a similar transcriptional signature indicates efficacy of the test compound for treating pain.

According to some embodiments of the invention, the importin alpha3 deficient neuronal cell is an importin alpha3 null cell.

According to some embodiments of the invention, the neuronal cell is a dorsal root ganglion cell.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D demonstrate balance, coordination and pain responses in importin α knockout mice. FIG. 1A is a graph demonstrating the results of rotarod tests which show significant balance and coordination deficits in importin α3 and α4 null mice. FIG. 1B is a graph demonstrating the results of pole tests which reveal increased time to turn (T_{Turn}) on the vertically-oriented pole for importin al and α3 null animals. FIG. 1C is a graph demonstrating the results of wire-hanging tests which highlight decreased latency to fall for importin α3 and importin α4 null mice. FIG. 1D is a graph of paw-licking latency time which shows that capsaicin (C) injection to paw pads reveal differences in paw-licking latency in treated WT (+/+) mice compared to vehicle controls (V), but no such difference in importin α3 null animals (−/−). All data is shown as mean±SEM. n≥5 animals for each genotype per test. * p<0.05; ** p<0.01; *** p<0.001, **** p<0.0001, two-tailed t-test (FIGS. 1A-C), or one-way ANOVA followed by Tukey's multiple comparison test (FIG. 1D).

FIGS. 2A-E demonstrates assessment of acute and chronic pain responses in importin α3 knockout mice. FIG. 2A is a graph demonstrating response to a heat probe. In contrast to the other importin α knockout lines, importin α3 (α3) knockout mice displayed a higher latency of paw withdrawal in response to noxious heat stimulus (58° C.) compared to wild type (WT) littermates. n=7-24. **** indicates p<0.0001, in one way ANOVA followed by Tukey's multiple comparison test. FIG. 2B is a graph of paw-licking latency time which shows that capsaicin-injected WT (+/+) mice had an increased response compared to vehicle-treated animals (V), while no difference was seen in importin α3 null (−/−) animals. n=10-13. * indicates p<0.05, in one-way ANOVA followed by Tukey's multiple comparison test. FIG. 2C is a schematic representation of the spared nerve injury model (SNI). FIG. 2D is a graph demonstrating the effects of gabapentin in the spared nerve injury (SNI) model of neuropathic pain. Gabapentin (100 mg/kg) was administered by intraperitoneal injection two months following establishment of the model, and then again one week later. Gabapentin-treated animals showed significant amelioration of the phenotype, as assessed by paw withdrawal threshold (PWT) in Von Frey tests. FIG. 2E is a graph demonstrating paw withdrawal threshold (PWT) as assessed by the Von Frey test in SNI animals. Importin α3 null (−/−) mice recover from 60 days onwards and reveal significant improvement from day 74 onwards. n≥5 animals for each genotype per test. * p<0.05; ** p<0.01; **** p<0.0001, two-way ANOVA followed by Sidak's multiple comparison test. All data is shown as average ±SEM.

FIGS. 3A-H demonstrate validation of the results obtained in the importin α3 null mice using AAV9 viral constructs for delivery of importin α3 shRNA. FIG. 3A is western blot analysis of importin α3 (Impα3) and GAPDH in protein extracts from HEK cells transduced with AAV9 expressing control shRNA (shCtrl), Importin α3 shRNA (shα3) for knockdown, GFP or α3 overexpression (α3OE) constructs. FIGS. 3B-C demonstrate the quantification of impα3 from FIG. 3A showing downregulation in shα3-treated cells compared to shCtrl (FIG. 3B), or upregulation in α3OE-expressing cells compared to GFP (FIG. 3C). GADPH served for normalization in both cases. FIG. 3D are microscope images of DRG sections from wild-type (+/+) or importin α3 null (−/−) mice one month following intrathecal injection with the indicated viral vectors co-expressing eGFP and the indicated shRNA, immunostained as indicated. Scale bar is 40 μm. FIG. 3E shows line scan measurements of intensity which reveal a reduction of impα3 signal in shα3-treated (+/+shα3, n=204) wild type animals compared to shCtrl (+/+ shCtrl, n=210). Also shown is importin α3 knockout animals transduced with shCtrl (−/− shCtrl, n=5).

FIG. 3F shows RT-qPCR quantification from DRG cultures from mice one month following intrathecal injection of the viral constructs using shCtrl versus sh importin α3 (shα3) which show significant downregulation of impα3 mRNA in shα3-treated animals compared to shCtrl. Results are shown as log 2 fold-change; normalized to GFP. FIG. 3G are microscope images of DRG neurons from cultures of shα3-treated animals compared to shCtrl immunostained as indicated, scale bar 40 μm. FIG. 3H is a quantification of impα3 intensity from FIG. 3G showing a significant reduction in nuclear levels in shα3-treated animals. All data is shown as mean±SEM; ** p<0.01; **** p<0.0001, unpaired two-tailed t-test.

FIGS. 4A-E demonstrate pain responsiveness following acute knockdown of importin α3. FIG. 4A is a graph demonstrating a reduced response of WT mice to noxious heat following intrathecal injection of AAV9 expressing an impα3 targeting shRNA (Shα3) as compared to mice injected with a control shRNA (ShCtrl). n=20. FIG. 4B is a graph demonstrating AAV9-driven overexpression of impα3 (α3 OE) restored heat sensitivity in impα3 knockout (−/−) animals, while eGFP overexpression had no such effect. n=4-8. FIG. 4C is a schematic representation of the timeline of viral knockdown followed by SNI. FIG. 4D shows photomicrographs of paws from SNI animals which reveal differences in paw aspect and clenching between mice that received control shRNA (shCtrl) versus Shα3 injected animals (left). Also shown are graphs of footprint width measurements from the Catwalk test which show a significant recovery by 60 days in Shα3-treated animals compared to ShCtrl-treated animals. n=7. FIG. 4E is a graph demonstrating paw withdrawal threshold (PWT) as assessed by the Von Frey test in SNI animals. The Von Frey tests reveal recovery in Shα3-treated mice as compared to ShCtrl-treated mice. All data is shown as mean±SEM. * p<0.05, ** p<0.01, *** p<0.001, **** p<0.0001, two-tailed unpaired t-test (FIGS. 4A, B and D) or two-way ANOVA followed by Sidak's multiple comparison test (FIG. 4E).

FIGS. 5A-D demonstrated behavioral tests following transduction with the AAV9 viral shRNA constructs. Wild-type mice injected intrathecally with control shRNA (shctrl) importin α3 shRNA (shα3) were tested three weeks later in an open field (FIG. 5A) and rotarod (FIG. 5B). No deficits were observed in either test. Importin α3 null mice (−/−) injected with the same constructs were tested for sensitivity to noxious heat (FIG. 5C) and on the rotarod (FIG. 5D). No further alteration of the noxious heat response in importin α3 null animals treated with shα3 could be observed. n≥7 animals for each group/genotype and per test. All data is presented as mean±SEM. Statistical analysis was effected by two-way ANOVA followed by Sidak's multiple comparison test (FIGS. 5A, B and D) or two-tailed unpaired t-test (FIG. 5C).

FIGS. 6A-K demonstrate transcriptome analyses and the involvement of c-Fos nuclear import by importin α3 in mediating pain responses. FIG. 6A is a heat map representation of z-score transformed normalized expression values for 164 differentially expressed genes (DEG) between importin α3 null (−/−) and WT (+/+) DRGs (n≥4 mice per group). FIG. 6B represents an FMatch (geneXplain) identification of transcription factor binding sites (TFs) enriched in differentially expressed gene (DEG) promoters from importin α3 null DRGs. FIG. 6C is a heat map representation of z-score transformed normalized expression values for 530 differentially expressed genes (DEG) comparison between importin α3 null (−/−) and WT (+/+) DRGs adult tissue 7 days versus 2.5 months after injury (n≥3 mice per group). FIG. 6D shows TF families whose binding sites were shown to be enriched in promoters of the upregulated DE genes (161 genes) and downregulated genes (369 genes). FIG. 6E details the Transcription factors (TF) included in the AP1 family highlighted in the analysis. FIGS. 6F-G show nuclear localization of c-Fos, as quantified by line scan measurement, indicating that c-Fos nuclear localization is reduced in importin α3 null (−/−) DRG sections compared to WT (+/+). Scale bar 10 μm. Shown are representative results from three independent experiments. FIG. 6H-I show nuclear localization of c-Fos in dissociated adult DRG neurons in culture, which show a significant reduction of c-Fos nuclear localization in importin α3 null (−/−) neurons. Scale bar 50 μm. n=3 or n>67 neurons quantified for each treatment from 3 independent experiments. **** p<0.0001, two-tailed unpaired t-test. FIGS. 6J-K show graphs of responses to noxious heat following administration of the c-FOS inhibitor T-5224 (20 mg/kg, i.p.). T-5224 reduced the response to noxious heat in WT (+/+) mice, but had no additional effect in importin α3 null (−/−) mice. n=6-8. * p<0.05, ANOVA followed by Tukey's multiple comparison test. All data is represented as mean±SEM.

FIGS. 7A-B show the effect of the c-Fos inhibitor T-5224 on noxious heat response. T-5224 was injected i.p. to wild-type mice at the indicated doses, with assessment of paw withdrawal threshold (PWT) in response to noxious heat over eight days post-treatment (FIG. 7A). As the most marked effect was observed one day following injection of a dose of 10 mg/kg the test was repeated at the indicated doses and PWT was assessed one day following injection. All data is shown as mean±SEM. n>8 animals for each experimental group and per test. *** p<0.001, ANOVA followed by Tukey's multiple comparison test.

FIGS. 8A-D demonstrate an in silico screen for drugs mimicking the transcriptional effects of importin α3 loss which reveals new candidate analgesics. Importin α3 KO DEG lists were used to query CMap for small molecules with similar transcriptome effects. FIG. 8A is a graph demonstrating responses of WT mice to noxious heat 1 hour following i.p. injection of Sulmazole (0.5 mg/kg, n=5) or sulfamethizole (1.25 mg/kg, n=12) as compared to vehicle (5% DMSO in PBS, n=9). FIG. 8B is a graph demonstrating significant effects of sulmazole (1.25 mg/kg, n=6), and sulfamethizole (3.12 mg/kg, n=4) compared to vehicle (5% DMSO in PBS, n=5) in the SNI model of neuropathic pain. Drugs were tested 60 days following establishing the model by two i.p. injections with 1 week interval, followed by assessment of paw withdrawal threshold (PWT) by the Von Frey test. FIGS. 8C-D show representative immunostaining microscopy photographs (FIG. 8C) and quantitation (FIG. 8D) of c-Fos nuclear localization in DRG neurons cultured following SNI. Significant reductions in c-Fos nuclear localization was observed following treatment with Sulmazole (0.5 mg/kg) or Sulfamethizole (1.25 mg/kg), compared to vehicle control. Scale bar 25 μm. n>31 neurons quantified for each treatment from 3 independent experiments. Data is shown as mean±SEM (FIG. 8A-B) or SD (FIG. 8C), * p<0.05, ** p<0.01, **** p<0.0001, ANOVA followed by Tukey's multiple comparison test (FIGS. 8A, B and D).

FIG. 9 is a graph demonstrating quantification of c-Fos nuclear localization following drug treatment in Importin α3 knockout animals. c-Fos nuclear localization was quantified in DRG neurons cultured following SNI treated with sulmazole (0.5 mg/kg) or sulfamethizole (1.25 mg /kg), compared to vehicle control. Data is shown as mean±SEM, n>19 neurons for each treatment from three independent experiments, * p<0.05, ** p<0.01, **** p<0.0001, ANOVA followed by Tukey's multiple comparison test.

FIGS. 10A-D demonstrate reduced sensitivity to noxious stimuli in importin α3 mice. FIG. 10A is a graph demonstrating reduced heat sensitivity in importin α3 knockout mice, as determined by hot plate assays effected at 52, 55 and 58° C. n≥10, ** indicates p<0.005, *** indicates p<0.001; **** indicates p<0.0001, ANOVA followed by Tukey's multiple comparison test. FIG. 10B is a graph demonstrating reduced cold sensitivity in importin α3 null mice, as determined by acetone tests. n≥15, * indicates p<0.05, two-tailed unpaired t-test. FIG. 10C is a graph demonstrating no differences in basal mechanosensitivity measured as paw withdrawal threshold (PWT) in the Von Frey test in importin α3 null versus wild type mice. n≥9. FIG. 10D is a graph demonstrating no differences in mechanosensitivity between importin α3 null and wild type mice as measured by PWT in the Von Frey test one hour following injection of capsaicin. n≥5, Kruskal-Wallis test followed by Dunn's multiple comparison test, * indicates p<0.05. Data is shown as mean±SEM.

FIG. 11 are representative paw images from SNI animals demonstrating recovery of paw morphology and reduced clenching in importin α3 knockout versus wild type animals.

FIGS. 12A-H demonstrate validation of peripheral neuron specificity of AAV-PHP.S viral constructs. Immunostaining for TuJ1 and GFP from spinal cord (lumbar section) and DRG of mice 6 weeks following intrathecal injection with AAV-PHP.S expressing GFP and either shCtrl (FIG. 12A-C) or shα3 (FIGS. 12D-F). FIGS. 12B and 12E are enlargements from the ventral horn area in FIGS. 12A and 12D, respectively. Scale bars, FIG. 12D 150 μm, FIGS. 12E-F 100 μm. FIGS. 12G-H show graph demonstrating percentage of GFP-positive neurons in the lumbar ventral horn (FIG. 12G) and L4 DRGs (FIG. 12H). n≥6 per group. Data is shown as mean±SEM.

FIGS. 13A-D demonstrate the effect of importin α3 knockdown by AAV-PHP.S delivery of shRNA in the SNI model of neuropathic pain. FIG. 13A is a schematic representations of timeline for shRNA-mediated knockdown by intrathecal injection of AAV-PHP.S in SNI. FIG. 13B is a graph demonstrating PWT in SNI animals treated with AAV-PHP.S shRNA against importin α3 (shα3) or scrambled control shRNA (shCtrl). n=9, * indicates p<0.05; ** indicates p<0.01; *** indicates p<0.001, **** indicates p<0.0001, two-way ANOVA followed by Sidak's multiple comparison test. FIG. 13C is a graph demonstrating spontaneous (unevoked) paw licking duration measured at 1 week (baseline) and 12 weeks following SNI. n≥9 per group. * indicates p<0.05, ** indicates p<0.01, Kruskal-Wallis followed by Dunn's multiple comparison tests. FIG. 13D shows representative paw images demonstrating a recovery of the paw morphology and reduced clenching in importin α3 knockdown versus control shRNA treated animals. Data is shown as mean±SEM.

FIG. 14 is a graph demonstrating expression levels of four AP1-target genes, Syngap1, Slc38, Gpr151 and Rtl1, as determined by RT-qPCR analysis comparing expression levels at one versus 11 weeks following SNI in wild-type and importin α3−/− DRGs. n=3, * indicates p<0.05, ** indicates p<0.01, one-way ANOVA followed by Sidak's multiple comparison test.

FIGS. 15A-G demonstrate c-Fos expression and interaction with Importin α3. FIG. 15A shows representative images of DRG neurons harvested from ganglia 4 hours following SNI and cultured for 24 hours prior to immunostaining for c-Fos, TRPV1 and DAPI. Scale bar 100 μm. FIG. 15B is a graph demonstrating quantification of c-FOS in nucleus and cytoplasmic compartments of TRPV1-positive neurons from the cultures shown in A. n=117, **** indicates p<0.0001, Unpaired two-tailed t-test. FIG. 15C shows representative images of L4 DRG section immunostained for importin α3, TuJ-1 and MBP. Scale bar 10 μm. FIG. 15D is a western blot analysis of N2a cells transfected with BioID fusion proteins, YFP-miniTurbo and importin α3-miniTurbo. Biotinylated proteins were affinity purified after 6 hours incubation of the cultures with 500 μM biotin and subjected to Western blotting. Blots were probed for c-Fos, importin α3, and importin β1. FIG. 15E shows western blot analysis of DRG neurons from wild type (+/+) and importin α3 knockouts (−/−) cultured for 24 hours prior to Western blot analyses as shown. FIG. 15F shows graphs demonstrating quantification of the blots shown in FIG. 15E, normalized to GAPDH protein levels. n=3, data normalized to wild-type control. **** indicates p<0.0001, one-tail t-test. FIG. 15G shows representative images demonstrating reduced nuclear localization of c-Fos in DRG neurons from sectioned ganglia of importin α3 null compared to wild type mice. Immunostaining for TuJ-1, DAPI, c-Fos. Scale bar 10 μm. Data is shown as mean±SEM.

FIGS. 16A-B demonstrate importin α3 and c-Fos interaction, as determined by proximity ligation assay (PLA). FIG. 16A shows representative images of PLA for c-Fos and importin α3 in CGRP positive DRG neurons fixed following 24 hours in culture from both naive and injury groups. PLA signals are shown in red. Scale bar 30 μm. FIG. 16B is a graph demonstrating quantification of the number of PLA signals per neuron. n≥29 neurons per group from three independent experiments, **** indicates p<0.0001,* indicates p<0.05, ANOVA followed by Tukey's multiple comparison test.

FIG. 17 shows representative images demonstrating reduced nuclear localization of c-Fos in DRG neurons from sectioned ganglia of importin α3 null compared to wild type mice. Cell body and nucleus boundaries determined by Tuj-1 and DAPI staining as indicated (see also FIG. 15G). Scale bar 10 μm.

FIG. 18 is a graph demonstrating PWT in animals treated with 10 mg/kg T-5224 one week following SNI, and assessed by the Von Frey test at the indicated time points following treatment. n=8, *** indicates p<0.001, **** indicates p<0.0001. Kruskal-Wallis followed by Dunn's multiple comparison tests. Data is shown as mean±SEM.

FIGS. 19A-G demonstrate nuclear localization of c-Fos or c-Jun in shRNA-treated neurons in culture. Nuclear localization of c-Fos (FIGS. 19A-D) or c-Jun (FIGS. 19E-G) quantified in cultured adult DRG neurons transduced with AAV9 expressing shRNAs as indicated. n>28, scale bar 100 μm, *** indicates p<0.001, **** indicates p<0.0001, ANOVA followed by Tukey's multiple comparison test. Data is shown as mean +/− SEM.

FIGS. 20A-F demonstrate that acute knockdown or dominant-negative inhibition of AP-1 transcription factors attenuates chronic pain after SNI. FIG. 20A is a graph demonstrating reduced noxious heat responses in mice after intrathecal AAV9 delivery of shRNAs targeting c-Fos (shFOS1, shFOS2) or c-Jun (shJUN). n>4. * indicates p<0.05, *** indicates p<0.001, **** indicates p<0.0001, ANOVA followed by Dunnett's multiple comparison test. FIG. 20B is a graph demonstrating reduced mechanosensitivity in shJUN, but not shFOS, treated animals. n>4, ** indicates p<0.01, ANOVA followed by Dunnett's multiple comparison test. FIG. 20C is a graph demonstrating paw withdrawal threshold (PWT) assessed by the Von Frey test in SNI animals treated with the indicated shRNAs (shFOS indicates a mixture of both). n>5, ** indicates p<0.01, *** indicates p<0.001, two-way ANOVA. FIGS. 20D-E are graphs demonstrating that AAV9 overexpression of the A-Fos dominant-negative (DN) under the neuron-specific human SynapsinI promoter reduces noxious heat responses (FIG. 20D) without effects on basal mechanosensitivity (FIG. 20E). n>6, two-tailed unpaired t-test. FIG. 20F is a graph demonstrating PWT in SNI animals treated with the A-Fos dominant-negative (DN) construct. n>6, two-way ANOVA. Asterisks indicate significant treatment effects between the groups. ** indicates p<0.01, *** indicates p<0.001. Data is shown as mean±SEM.

FIGS. 21A-B demonstrate dose dependent effects of sulmazole and sulfamethizole treatments on mechanosensitivity. Paw withdrawal threshold was assessed by the Von Frey test one hour following drug treatment. Animals were injected i.p. one week following establishing SNI with the indicated concentrations of sulmazole (FIG. 21A) or sulfamethizole (FIG. 21B). n≥4, * indicates p<0.05, ** indicates p<0.005, Kruskal-Wallis followed by Dunn's multiple comparison tests. Data is shown as mean±SEM.

FIGS. 22A-C demonstrate time dependent effects of sulmazole and sulfamethizole treatments on Mechanosensitivity. FIG. 22A-B are graphs demonstrating duration of drug effects one week after SNI, with Von Frey tests performed 1, 5 and 24 hours following i.p. injection. n≥4, Kruskal-Wallis test followed by Dunn's multiple comparison test, *** indicates p<0.001. FIG. 22C is a graph demonstrating noxious mechanosensitivity testing of SNI mice treated as shown, using Von Frey filaments of 2 grams force. Scoring from 0 to 2, with 0=no response, 1=signs of discomfort, 2=withdrawal of the leg. n≥6, Kruskal-Wallis test followed by Dunn's multiple comparison test, * indicates p<0.05.

### DESCRIPTION OF SPECIFIC EMBODIMENTS OF THE INVENTION

The present invention, in some embodiments thereof, relates to methods of treating pain.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Pain, and particularly chronic pain, is currently one of the most common unmet medical needs, due to limited analgesic efficacy of existing drugs, coupled with adverse side effects. Globally, it has been estimated that 1 in 5 adults suffer from pain and that another 1 in 10 adults are diagnosed with chronic pain each year.

Importins are a group of proteins that transport protein molecules into the nucleus by binding to specific recognition sequences, called nuclear localization sequences (NLS). Importin polypeptide has two subunits, importin α and importin β, wherein members of the importin-3 subfamily can bind NLS cargo proteins as homodimers or can form heterodimers with importin-α. There are 6-7 importin α family members in any given mammal and individual cell types express different subsets of this ensemble in a tightly regulated manner.

Whilst reducing specific embodiments of the present invention to practice, the present inventors have now uncovered that importin α3 controls nociceptive and neuropathic pain and demonstrate that downregulating importin α3 indeed has an analgesic effect. In addition, the present inventors have also uncovered that downregulation of importin α3 results in differential expression of multiple genes and that drugs mimicking the transcriptional signature of loss of the importin α3 are endowed with analgesic effects as well.

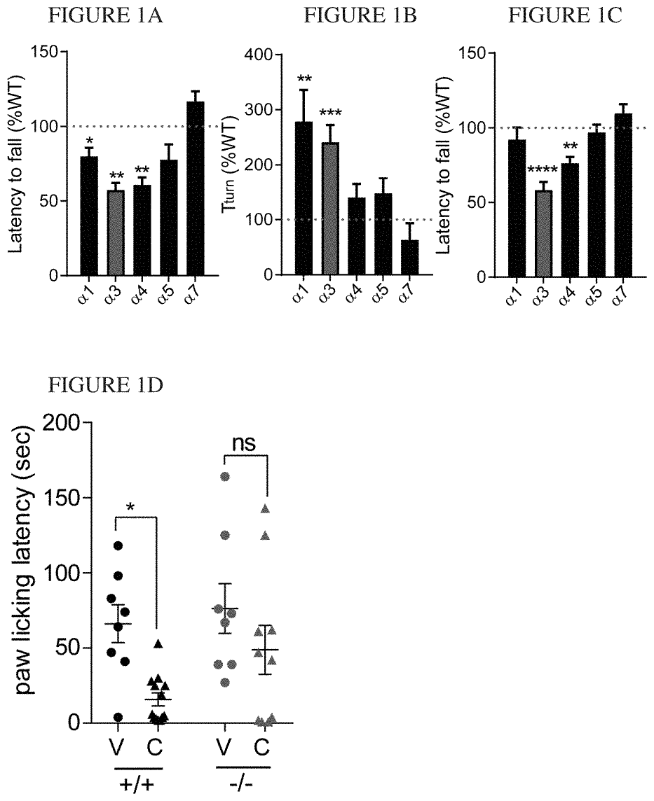

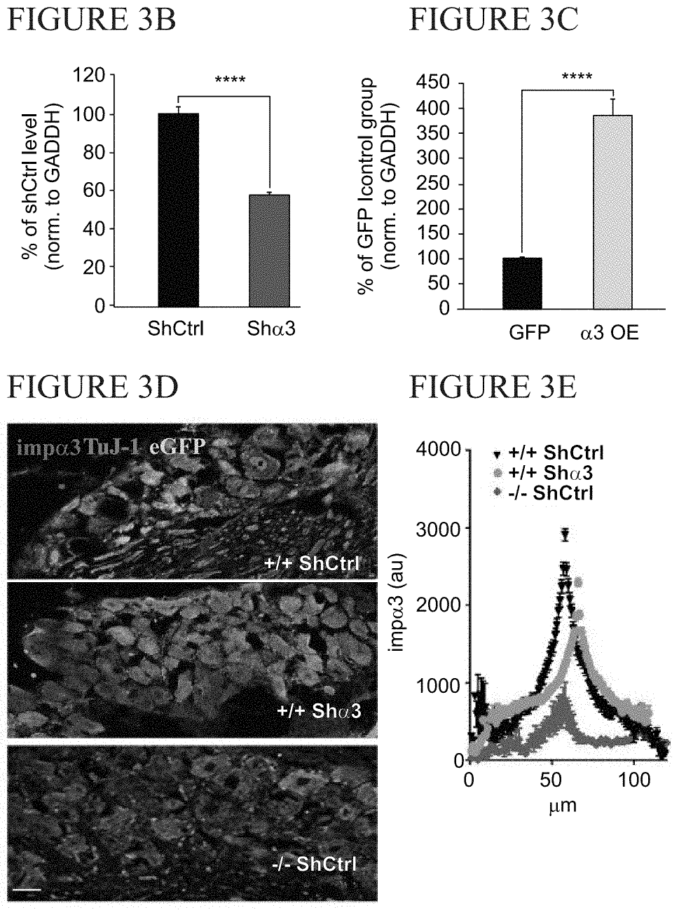

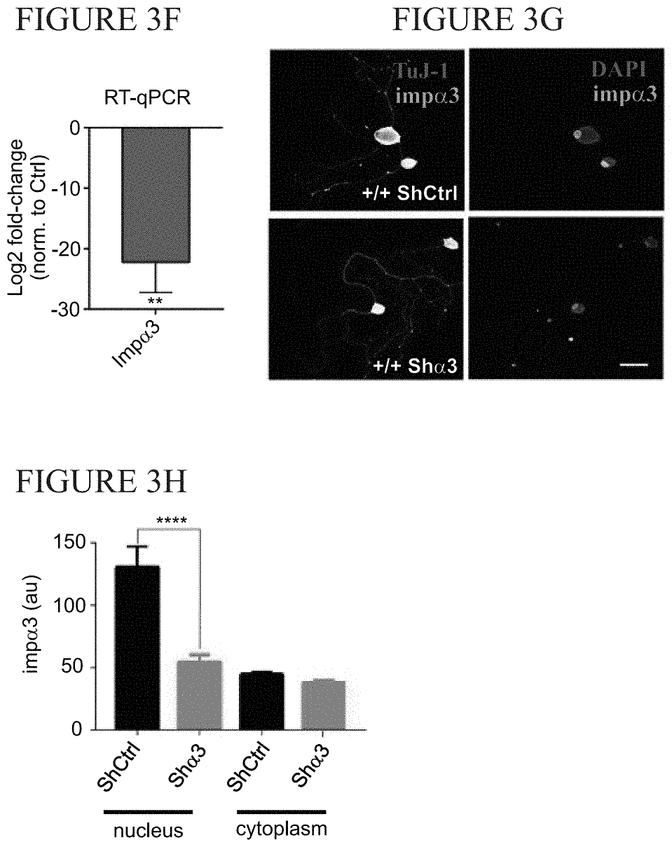

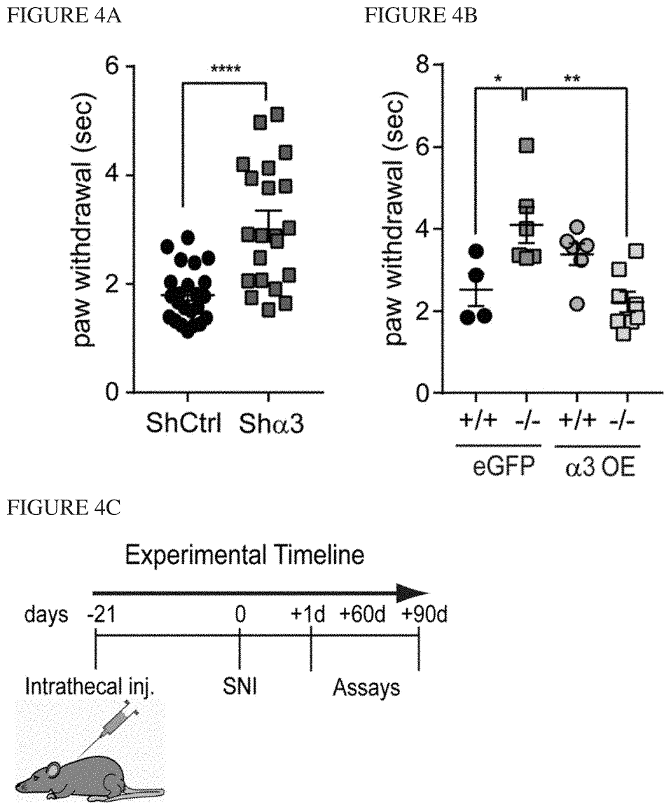

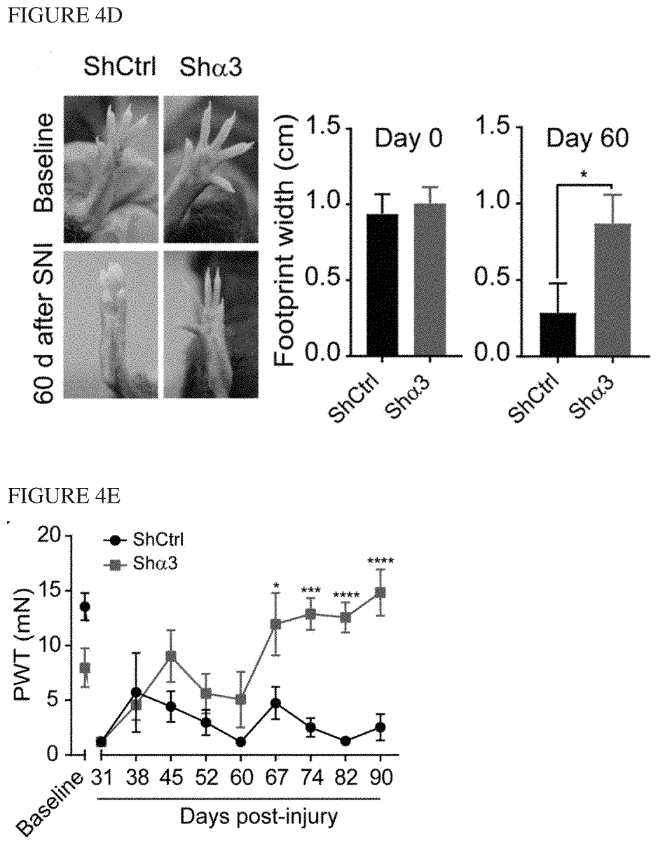

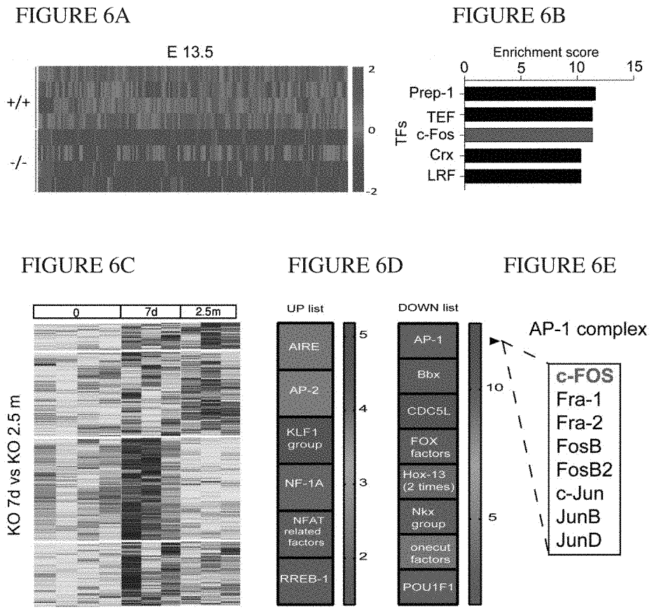

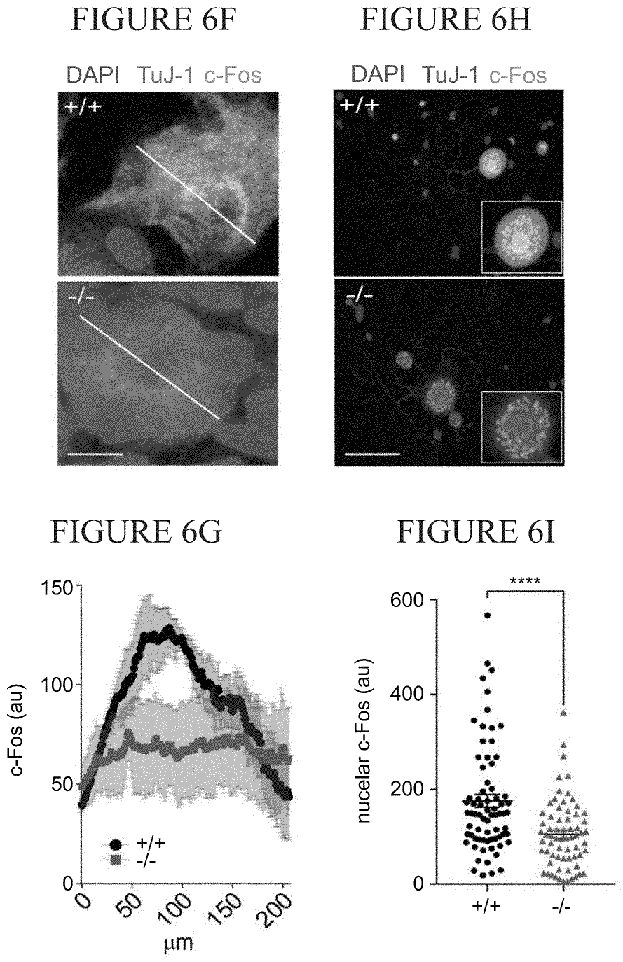

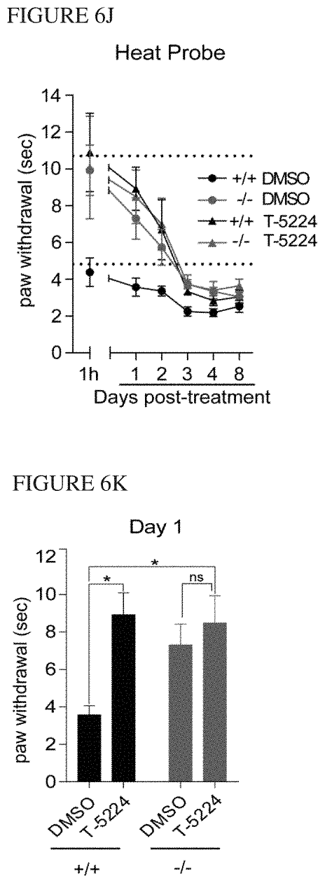

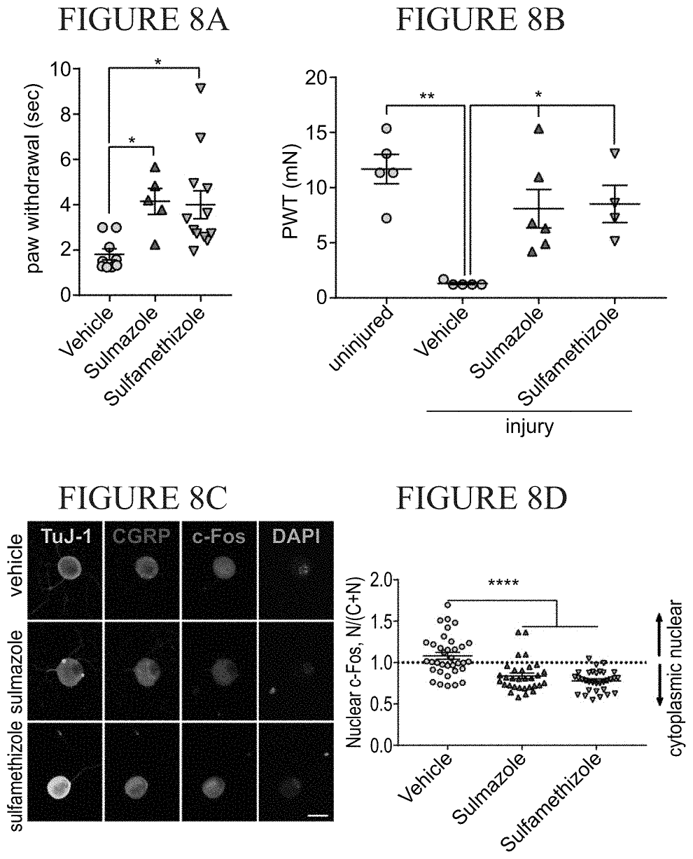

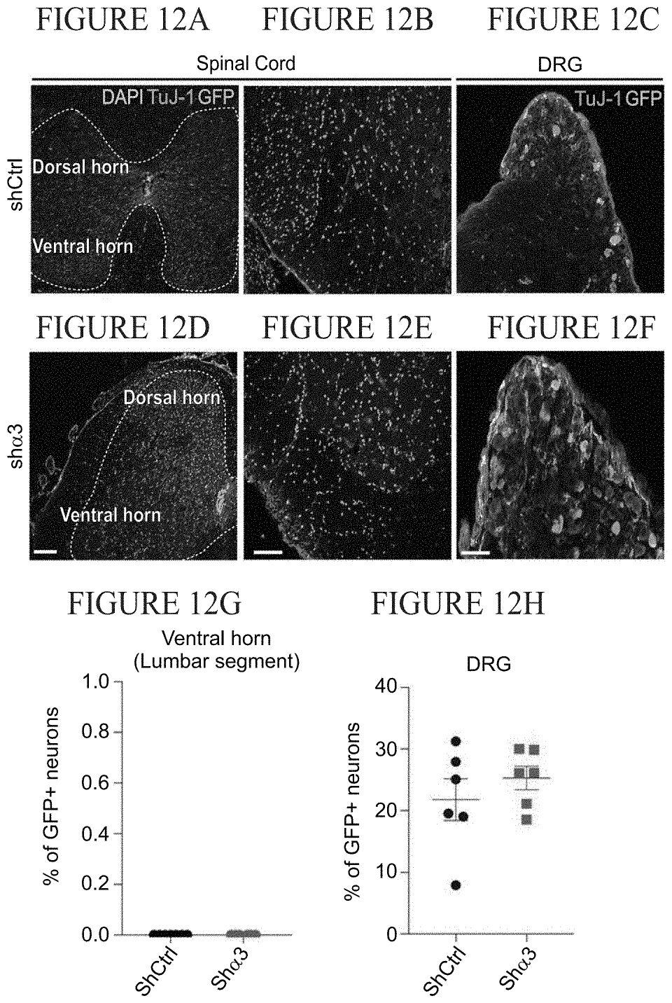

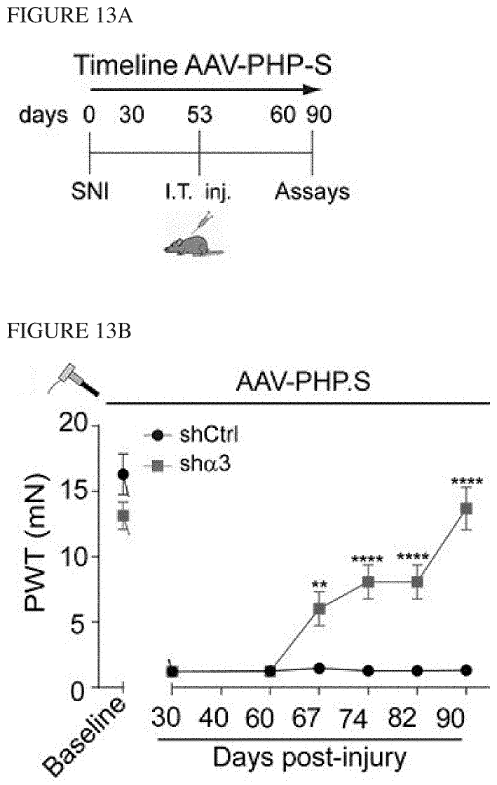

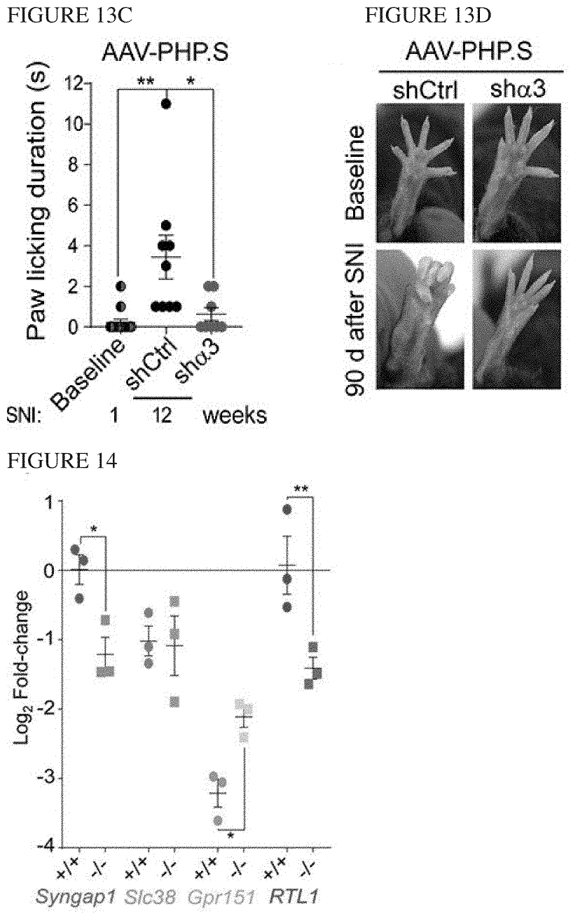

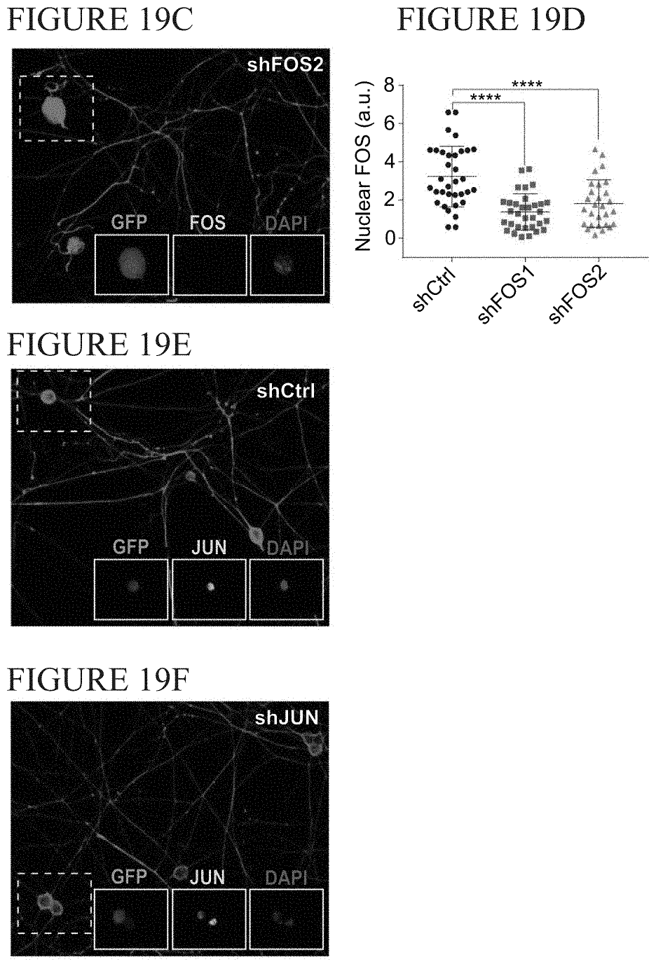

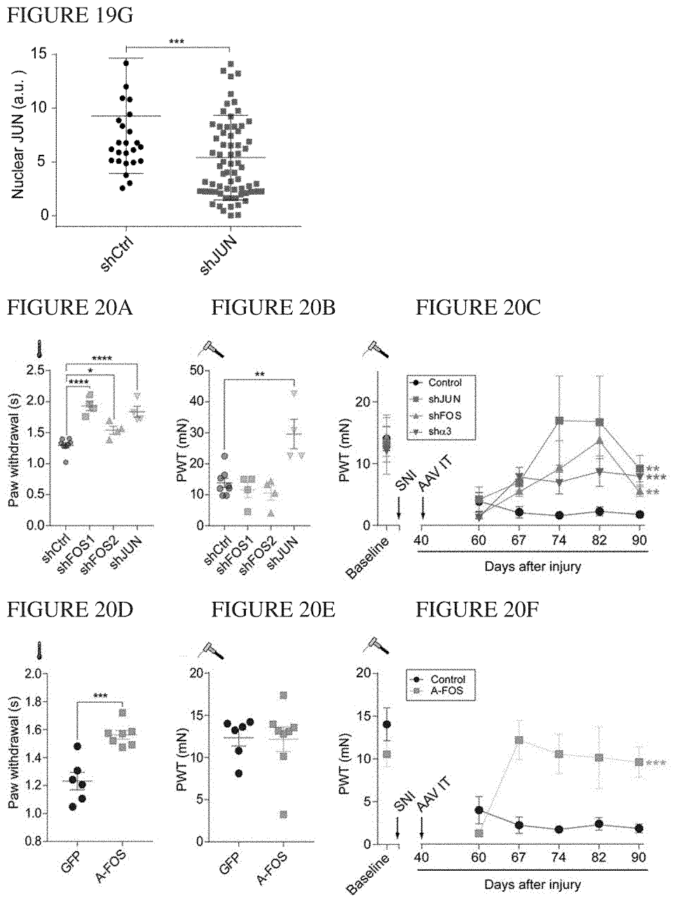

As is illustrated hereinunder and in the examples section, which follows, the present inventors show that importin α3 knockout (KO) mice present reduced sensitivity to noxious heat, chemically-induced and neuropathic pain (Example 1, FIGS. 1A-2E, 10A-D and 11). Following, these finding were corroborated in an acute knockdown model in adult animals using importin α3 shRNA (Example 1, FIGS. 3A-B, 4A, 4C-E, 5A-B and 6F-G). In addition, the present inventors show that induced expression of importin α3 increased pain responsiveness in the importin α3 knockout mice (Example 1, FIG. 4B). While elucidating the mechanisms underlying the observed reduction in pain sensitivity, the present inventors demonstrate that the effect of importin α3 on neuropathic pain arise specifically in sensory neurons (Example 2, FIGS. 13A-D). Moreover, the present inventors demonstrate significant changes in the expression of multiple genes and transcription factors in the importin α3 KO mice (Example 2, FIGS. 6A-E, 14-16B). Immunostaining further showed a significant reduction of c-Fos nuclear localization in sensory neurons of KO mice; and treatment with a c-Fos inhibitor (T-5224) reduced sensitivity to noxious heat in wild-type mice while it had no effect in importin α3 KO mice (Example 2, FIGS. 6F-K, 7A-B, 15G and 17-18). Without being bound by theory, these data suggest that the analgesic effect of importin α3 depletion is at least partially due to perturbation of the nuclear import of c-Fos. Further corroborating the involvement of the AP-1 pathway, the present inventors show that knock-down of c-Fos or c-Jun reduced sensitivity to noxious heat, chemically-induced and neuropathic pain (Example 2, FIGS. 19A-20F). In addition, based on the importin α3 KO mice transcriptome, the present inventors were able to identify multiple drugs having similar transcriptional effects as the importin α3 KO, and demonstrate that indeed several of these drugs (sulmazole and sulfamethizole) have analgesics and c-Fos localization effects (Example 3, FIGS. 8A-D and 21A-22C).

Based on the above, specific embodiments suggest that targeting importin α3 or any of the identified differentially expressed genes; and/or each of the identified drugs, can be used for treating pain, and more particularly, nociceptive and neuropathic pain.

Thus, according to an aspect of the present invention, there is provided a method of treating nociceptive or neuropathic pain in a subject in need thereof, the method comprising administering to the subject a therapeutically effective amount of an agent which binds importin α3 or a polynucleotide encoding same and inhibits expression and/or activity of said importin α3, thereby treating the nociceptive or neuropathic pain in the subject.

According to an additional or an alternative aspect of the present invention, there is provided an agent which binds importin α3 or a polynucleotide encoding same and inhibits expression and/or activity of said importin α3 for use in treating nociceptive or neuropathic pain in a subject in need thereof.

According to an additional or an alternative aspect of the present invention, there is provided a method of treating pain in a subject in need thereof, the method comprising administering to the subject a therapeutically effective amount of an agent capable of inhibiting expression and/or activity of a target selected from the targets listed in Table 1 hereinbelow.

According to an additional or an alternative aspect of the present invention, there is provided an agent capable of inhibiting expression and/or activity of a target selected from the targets listed in Table 1 hereinbelow for use in treating pain in a subject in need thereof.

TABLE 1

| Gene | Gene | Gene | Gene | Gene | Gene |
|---|---|---|---|---|---|
| Symbol | Symbol | Symbol | Symbol | Symbol | Symbol |
| Kpna4 | Shank1 | Fcrls | Sprr2j-ps | Cacna2d1 | Slfn9 |
| Gsx1 | 2210039B01Rik | Fam184b | Rnpc3 | Ildr2 | Tmem132b |
| Gm3893 | Sall1 | Wt1 | Alpk1 | Clcf1 | Gm12966 |
| Robo3 | Ldb3 | Phf21b | Nudt17 | Myh6 | Tia1 |
| Rhbg | Myh7b | Il13ra1 | Col24a1 | Hectd2 | Reg3b |
| Hoxc11 | Nrap | Gsdma | Mmp16 | Chrnb1 | Klf12 |
| Fgf15 | Smyd1 | Sema6a | Clca1 | Tmtc4 | 5930412G12Rik |
| Hotair | Nexn | Crybg1 | Plin2 | Zmym6 | Slfn10-ps |
| Hc | Krt15 | Fabp7 | Pde4b | Fam227a | 9030624G23Rik |
| Slc6a5 | Rmst | Egfr | Tnfrsf8 | Mapk6 | Sh2d1b2 |
| Gulo | Gpr179 | Phlda1 | Ephb2 | Gpr151 | Dok6 |
| Hoxd11 | Bhlhe22 | Aldh112 | Cyp4b1 | Fgf11 | Pappa2 |
| Slc32a1 | Alk | Igfbp3 | Stil | Tubalc | Tmem88b |
| BC024386 | Tnnt1 | Myo19 | Sesn2 | Olfr78 | Fam196a |
| Slc30a3 | Dtx1 | Sdc1 | Draxin | Zbtb37 | 4632427E13Rik |
| C130021I20Rik | Mapk13 | Pxdn | Nsg1 | Mdga1 | Mex3a |
| Slc27a2 | Inhbb | Pimreg | Cckar | Clec4a3 | Ccdc162 |
| Lbx1 | Slco1c1 | Slc6a4 | Gnpda2 | Adgrd1 | Scn2a |
| Skor1 | Gjd2 | Doc2b | Tes | Snhg11 | Wfdc3 |
| Gm17750 | Obscn | Bdkrb2 | Rasl11a | Lncpint | Fyb2 |
| Pax2 | Myom1 | Arg2 | Mtus2 | Liph | 5830417I10Rik |
| Nxph1 | Clec7a | Pcnx | Akr1b8 | Pard6b | Ccr5 |
| Gad1 | Ntn1 | Hhipl1 | Dusp11 | Plppr4 | St6galnac4 |
| Lhx1 | Perp | Meg3 | Chl1 | Slc38a6 | Spaca6 |
| Stra6l | Pak6 | Gadd45g | Aldh1l1 | Serpinb1a | Hist2h3c2 |
| Sall3 | Gdf10 | Ahrr | Grip2 | Pcdhb8 | Bambi-ps1 |
| Lhx5 | Tspan11 | Rasgrf2 | Ptpro | D830044D21Rik | Gm16185 |
| Aqp6 | Slc12a5 | Fst | Bcat1 | Chrm3 | Rtl1 |
| Dpep1 | Elfn2 | Stmn4 | Camk1 | Cd109 | Gm13571 |
| A530058N18Rik | Chl1 | Bmp1 | Siglece | Plaur | Ftx |
| Sst | Tmem132d | Fyb | Apba2 | Nexmif | A730081D07Rik |
| Lmx1b | Ankrd35 | Myo10 | Tmc5 | Tifa | Gm15751 |
| Cacng3 | Gpm6a | Shisa9 | Kif22 | Gap43 | 4732440D04Rik |
| Foxb1 | Fap | Ly6g | Zkscan2 | Tgif1 | Gm38399 |
| Lamp5 | Fibcd1 | Snai2 | Ptpn5 | Flrt2 | Malat1 |
| Pax8 | Fndc5 | Adamts1 | Dnhd1 | Nrip1 | Methig1 |
| Krt8 | Crabp1 | Stfa2 | Cckbr | Fndc9 | Gm9866 |
| Mab21l1 | Sult5a1 | Slc4a8 | Fgf3 | Pthlh | Gm38394 |
| Myh8 | Gramd1c | Cdkn1a | Slc7a3 | Ccr2 | Gm21781 |
| Lmo3 | Tnfrsf11a | Slc38a1 | Il2rg | Adamts16 | Gm9821 |
| Slc18a3 | Dlg2 | Kcnk16 | Anxa10 | Crh | A630023A22Rik |
| Krt14 | Lrrc4b | Celsr3 | Smad1 | Grem2 | Gm26788 |
| Dbx1 | Kcnj2 | Xdh | Slc12a3 | Mchr1 | Gm17275 |
| Cldn4 | Adamts19 | Rhoq | Aqp9 | Prag1 | Gm16793 |
| Irx6 | Slc10a4 | Epb4114a | Nktr | Abca12 | Rian |
| Myh7 | Slc16a14 | Grp | Trib3 | Neto1 | Mir124a-1hg |
| Chrnd | Adgrb2 | Anxa1 | Lmo7 | Sprr1a | 9530059O14Rik |
| Xirp1 | Kif26a | Fam111a | Galnt9 | Prokr2 | 9630001P10Rik |
| Gad2 | Bhlhe40 | Slc15a3 | Chst2 | Arhgap42 | Gm26917 |
| Gjd4 | Dnah2 | Lipk | Frrs1 | Trim15 | Gm26945 |
| Mlc1 | AW549542 | Rin1 | Mapkbp1 | Htr1f | Gm29374 |
| 5430431A17Rik | Fstl5 | Slc29a2 | Coil | Flrt3 | Gm4208 |
| Tacstd2 | Fndc1 | Gal | Ano7 | Pcdhb11 | Gm37233 |
| Wnt7b | Sorl1 | Fosl1 | Loxl2 | Prdm9 | Pcdhgb2 |
| Cartpt | Usp29 | Cyp26a1 | Igsf9b | Igfn1 | Gm7694 |
| Pdyn | Magi2 | Gfra1 | Cd207 | Cx3cr1 | Pcdhgb1 |
| Ttn | Shroom1 | Hps1 | Tet3 | Tchh | Gm37092 |
| Pax7 | Camk2n1 | Itga7 | Tpbg | Epha3 | Pcdha12 |
| Pou3f2 | Acbd4 | Adam8 | Ccl12 | Nav2 | 2210017I01Rik |
| Gabrp | Fgf9 | Podx1 | Kcnh4 | Hist1h1d | Pcdhgb4 |
| Lad1 | Rftn1 | Bach1 | Nfkbiz | Pcdh15 | Gm38020 |
| Krt5 | Fgfr3 | Pfkfb4 | Sstr1 | Glis3 | Gm20045 |
| 1110015O18Rik | Fblim1 | Col7a1 | Leng8 | Socs3 | Gm31831 |
| Dsp | H19 | Dhtkd1 | Chrna5 | Styx | 4932422M17Rik |
| Trp63 | Scpep1 | Il1r1 | Sbno2 | H2-T24 | AI506816 |
| D030068K23Rik | Cttnbp2 | Mstn | Zfp57 | Adm2 | Gm42664 |
| Krt17 | Gm38393 | Bard1 | Gadd45a | Oscar | Gm43843 |
| Barhl1 | Cfp | Ecel1 | Proser3 | Gabra5 | Gm2762 |
| Mybph | Gabrg1 | Dusp27 | Arhgap33 | Arntl | Gm34583 |
| Actn2 | Tubb6 | Pou2f1 | Inhbb | Soga1 | Gm43980 |
| Krt7 | Zim1 | Cr2 | Galnt6 | Mettl4 | Gm44168 |
| Grin2c | Rgs20 | Atf3 | Serpine1 | Nfil3 | Gm7972 |
| Casq2 | Il17ra | Lamb3 | Vgf | Scn3a | Gm45867 |
| Arx | Man1a | Ddr2 | Vash2 | Mettl7a3 | Gm30085 |
| Neb | Itpkc | Hsd17b7 | Tecta | Sez6l | C78859 |
| Myom2 | Stat5a | Acvr1c | Smim3 | Pde1a | Gm47133 |
| Esrp1 | Col5a3 | Acvr1 | Prdm1 | Reg1 | Btbd8 |
| Kremen2 | Pde1c | Cybrd1 | Slc22a23 | Plekhh1 | AC160637.1 |
| Plk5 | Wisp1 | Frmd5 | 1700003F12Rik | Ppef1 | Gm47593 |
| Hspb2 | Mthfd2 | Chac1 | Cyp24a1 | Phactr2 | Gm47595 |
| Car14 | Tyrp1 | Car1 | Atp5l | Sox7 | Gm47903 |
| Tnnt2 | Adamts4 | Car3 | Irs2 | Sox11 | E430024I08Rik |
| Chrdl2 | Phox2a | Mrgbp | Prc1 | Srrm4 | Tubb2a-ps2 |
| Fut9 | Mcoln2 | Procr | Srrm2 | Syngap1 | CT030170.4 |
| Pkp3 | Csf1 | Tnik | Rnf122 | Flnc | Gm48239 |
| Jsrp1 | Abca1 | Ptx3 | Ajap1 | Trp53i11 | |
| Slc38a5 | Fam163a | S100a11 | Cited2 | Gm5152 | |

According to specific embodiments, the target is Syngap1 or RTL1.

As used herein the term “Syngap1”, also known as Synaptic Ras GTPase-activating protein 1 or synaptic Ras-GAP 1 or SYNGAP1, refers to the polynucleotide or polypeptide expression product of the SYNGAP1 gene (Gene ID: 8831). According to specific embodiments, the Syngap1refers to the human Syngap1, such as provided in the following Accession Numbers: NM_006772, NM_001130066, NP_001123538, NP_006763.

As used herein the term “RTL1”, also known as retrotransposon like 1, refers to the polynucleotide or polypeptide expression product of the RTL1 gene (Gene ID: 388015). According to specific embodiments, the RTL1 refers to the human RTL1, such as provided in the following Accession Numbers: NM_001134888, NP_001128360.

According to an additional or an alternative aspect of the present invention, there is provided a method of treating pain in a subject in need thereof, the method comprising administering to the subject a therapeutically effective amount of an agent capable of enhancing expression and/or activity of a target selected from the targets listed in Table 2 hereinbelow.

According to an additional or an alternative aspect of the present invention, there is provided an agent capable of enhancing expression and/or activity of a target selected from the targets listed in Table 2 hereinbelow for use in treating pain in a subject in need thereof.

TABLE 2

| Gene | Gene | Gene | Gene | Gene | Gene |
|---|---|---|---|---|---|
| Symbol | Symbol | Symbol | Symbol | Symbol | Symbol |
| Six1 | Flt4 | Per3 | Nme5 | 8430408G22Rik | Serpina1e |
| Tbc1d9 | Smtn | Rbp7 | Mag | Rasd1 | Plekhf1 |
| Sh2d4b | Efcab10 | Nppb | Cdkn1c | Vwa3b | Cox7a1 |
| Kcnmb2 | Nr1d1 | Gabra4 | Egr2 | Serpinb1b | Gm10800 |
| Gm20752 | Ngb | Pxmp2 | Egr1 | Kctd19 | Abhd11os |
| Gm4349 | Nxnl2 | Gkn3 | Ciart | AU021092 | Gm13446 |
| Atp6v1c2 | Crhbp | Mfap5 | Thap3 | Kcnf1 | Gm15500 |
| Cd1d2 | Mroh2b | Ckm | Lrrc17 | Slc6a7 | Tbx3os2 |
| Tpd52l1 | Ly6h | Cpxm2 | Fmo2 | Prx | Hopxos |
| S100a4 | Gpihbp1 | Mrgprf | Plekha4 | Aldh1a1 | Pou3f1 |
| Gstt1 | Rfc4 | Tnni2 | Gstm2 | Ceacam10 | Gypc |
| Gstt3 | C1qtnf12 | Magix | Dlk1 | 22Rik2900041M | Apold1 |
| Fosb | Sox8 | Chrdl1 | Tmco4 | Klf2 | Lrrc32 |
| Nqo1 | Pdzph1 | Nkd1 | Pla2g5 | C1ra | 9130204L05Rik |
| Hlf | Rbp4 | Pllp | Cldn5 | Myh1 | Gm10717 |
| Nes | Arhgap19 | Comp | Mbp | Mpz | Gm2694 |
| Crip1 | Slc9a2 | Oaf | Reln | Capg | Gm26783 |
| Id3 | Des | Cryab | Id1 | Myh4 | Gm28875 |
| G0s2 | Efhd1 | Tagln | Ncmap | Dbp | Gm6204 |
| Cox4i2 | Bok | Gsta4 | Cdkn2a | Tmem40 | Gm42621 |
| Kif19a | Fgf7 | Mlip | Tnfaip8l1 | Mcpt4 | Gm42788 |
| Rdm1 | Syt13 | Bfsp2 | Fam178b | Rps7 | Gm5886 |
| Bcas1 | Olfml3 | Fhl3 | Lrrc75a | Tnnt3 | Gm35721 |
| Egfl8 | Vcam1 | Cryzl2 | Fam46b | Cys1 | |
| Hsd11b1 | Wdr31 | Scx | Prr15l | Klk8 | |
| Jph2 | Tpm2 | Emid1 | Chst9 | Cldn19 | |
| Kcnab3 | Tinagl1 | Cpne7 | Kcne4 | Fxyd6 | |
| Hal | Padi2 | Tbxa2r | Gjb1 | Myl9 | |

According to an additional or an alternative aspect of the present invention, there is provided a method of treating pain in a subject in need thereof, the method comprising administering to the subject a therapeutically effective amount of a compound selected from the compounds listed in Table 3 hereinbelow, thereby treating pain in the subject.

According to an additional or an alternative aspect of the present invention, there is provided a compound selected from the compounds listed in Table 3 hereinbelow for use in treating pain in a subject in need thereof.

TABLE 3

| Compound | Compound | Compound | Compound | Compound |
|---|---|---|---|---|
| AG-028671 | fludrocortisone | Minoxidil | dehydrocholic acid | LY-294002 |
| ajmaline | proguanil | valproic acid | propranolol | alclometasone |
| metronidazole | profenamine | Chlortetracycline | benserazide | piretanide |
| acetohexamide | sirolimus | cyclic adenosine | chlorzoxazone | 3- |
| adipiodone | meteneprost | monophosphate | metformin | acetamidocoumarin |
| pheneticillin | lithocholic acid | methylbenzethonium | boldine | oxolinic acid |
| pramocaine | propidium iodide | chloride | viomycin | terbutaline |
| prasterone | sulmazole | glycopyrronium bromide | tiratricol | ondansetron |
| dacarbazine | Prestwick-972 | ampyrone | trihexyphenidyl | quinpirole |
| gelsemine | CP-690334-01 | adenosine phosphate | procarbazine | metaraminol |
| MK-886 | betulin | Colistin | amylocaine | saquinavir |
| diphenylpyraline | ticlopidine | Prochlorperazine | pralidoxime | sulindac |
| vitexin | sulfadimethoxine | Benzocaine | meclofenamic acid | SC-58125 |
| ciclacillin | noretynodrel | 0317956-0000 | clonidine | estradiol |
| trimipramine | diphemanil | Citalopram | dimenhydrinate | bisoprolol |
| 0173570- | metilsulfate | Dipivefrine | co-dergocrine | physostigmine |
| 0000 | dirithromycin | Octopamine | mesilate | benzamil |
| sulfadimidine | azapropazone | Rilmenidine | AG-013608 | copper sulfate |
| sulfamethizole | Prestwick-692 | Paromomycin | 15-delta | |
| ceftazidime | oxyphenbutazone | Tocainide | prostaglandin J2 | |
| | | Dexamethasone | valdecoxib | |

According to specific embodiments, the compound is selected from the group consisting of sulmazole, sulfamethizole, ajmaline, pramocaine, prasterone, MK-886, diphenylpyraline, vitexin, ciclacillin, sulfamidine, ceftazidime and profenamine.

According to specific embodiments, the compound is selected from the group consisting of sulmazole, sulfamethizole, pramocaine, prasterone, MK-886, diphenylpyraline, vitexin, ciclacillin, sulfamidine, ceftazidime and profenamine.

According to specific embodiment, the compound is sulmazole or sulfamethizole.

“Sulmazole”, 2-(2-Methoxy-4-[methylsulfinyl]phenyl)-1H-imidazo(4,5-b)pyridine, CAS NO: 73384-60-8, can be obtained from e.g. Sigma-Aldrich.

“Sulfamethizole”, 4-Amino-N-(5-methyl-1,3,4-thiadiazol-2-yl)benzenesulfonamide, CAS NO: 144-82-1, can be obtained from e.g. Sigma-Aldrich.

According to specific embodiments, the compound is not ajmaline (CAS NO: 4360-12-7). The term “treating” or “treatment” refers to inhibiting, preventing or arresting the development of a pathology (disease, disorder or medical condition e.g. pain e.g. nociceptive pain, neuropathic pain) and/or causing the reduction, remission, or regression of a pathology or a symptom of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, e.g., human beings at any age and of any gender who suffer from the pathology. According to specific embodiments, this term encompasses individuals who are at risk to develop the pathology.

According to specific embodiments, the subject is not afflicted with an inflammatory disease.

According to specific embodiments, the subject is not afflicted with a vascular inflammatory disease (e.g., acute lung injury).

As used herein the term “pain” refers to all types of pain.

Non-limiting examples of pain include postherpetic neuralgia, diabetic neuropathy, pruritus, psoriasis, cluster headache, postmastectomy pain syndrome, rhinopathy, oral mucositis, cutaneous allergy, detrusor hyperreflexia, loin pain/hematuria syndrome, neck pain, amputation stump pain, reflex sympathetic dystrophy, pain due to skin tumor and arthritis including rheumatoid arthritis, osteoarthritis, headache, post-surgical pain, oral pain, pain caused by injury, vulvodynia, interstitial cystitis, rhinitis, burning mouth syndrome, oral mucositis, herpes neuralgia, dermatitis, pruritis, tinnitus, phantom or amputation stump pain, acquired immune deficiency syndrome neuropathy, back pain, opioid-resistant pain, visceral pain, bone injury pain, pain during labor and delivery, pain resulting from burns (including sunburn), post-partum pain, migraine, angina pain, genitourinary tract-related pain including cystitis.

According to specific embodiments, the pain is acute pain.

According to other specific embodiments, the pain is chronic pain.

According to specific embodiments, the pain is nociceptive pain.

As used herein, the term “nociceptive pain” involves direct activation of the nociceptors, such as mechanical, chemical, and thermal receptors, found in various tissues, such as bone, muscle, vessels, viscera, and cutaneous and connective tissue. Nociceptive pain occurs in the setting of an undamaged nervous system, e.g. the afferent somatosensory pathways are considered intact.

Non-limiting examples of nociceptive pain include post-operative pain, cluster headaches, dental pain, surgical pain, pain resulting from burns, sunburns, exposure to extremely cold temperatures, bruises, fractures, post-partum pain, angina pain, genitourinary tract related pain, damage by contact with toxic of hazardous chemicals.

According to specific embodiments, the pain is an acute thermal nociceptive pain or acute mechanical nociceptive pain.

According to specific embodiments, the pain is an acute chemically-induced pain.

According to specific embodiments, the pain is neuropathic pain.

As used herein the term “neuropathic pain” refers to pain initiated or caused by injury to or dysfunction of the central or peripheral nervous system. According to specific embodiments, the neuropathic pain has typical symptoms such as hyperesthesia (enhanced sensitivity to a natural stimulus), hyperalgesia (abnormal sensitivity to pain), allodynia (widespread tenderness, characterized by hypersensitivity to non-noxious tactile stimuli), and/or spontaneous burning pain.

Non-limiting examples of neuropathic pain include, but are not limited to, medication-induced neuropathy and nerve compression syndromes such as carpal tunnel, radiculopathy due to vertebral disk herniation, post-amputation syndromes such as stump pain and phantom limb pain, metabolic disease such as diabetic neuropathy, viral-related neuropathy including herpes zoster and human immunodeficiency virus (HIV) disease, tumor infiltration leading to irritation or compression of nervous tissue, neuritis, as after cancer radiotherapy, autonomic dysfunction from complex regional pain syndrome (CRPS), trigeminal neuralgia, postherpetic neuralgia, and the reflex sympathetic dystrophies including causalgia, mononeuropathies, peripheral nerve injury, central nerve injury, opioid resistant neuropathic pain, bone injury pain, pain during labor and delivery, non-specific lower back pain, multiple sclerosis-related pain, fibromyalgia, acute and chronic inflammatory demyelinating poly radiculopathy, alcoholic polyneuropathy, segmental neuropathy, ischemic optic neuropathy, geniculate neuralgia, occipital neuralgia, periodic migrainous neuralgia, chemotherapy-induced polyneuropathy, brachial plexus avulsion, post-surgical neuropathy including post-mastectomy pain or post-thoracotomy pain, idiopathic sensory neuropathy, nutrition deficiency-related neuropathy, phantom limb pain, post-radiation plexopathy, radiculopathy, for example, sciatica, toxin exposure-related neuropathy, post-traumatic neuralgia, compressive myelopathy, Parkinson's disease-related neuropathy, post-ischemic myelopathy, post-radiation myelopathy, post-stroke pain, post-traumatic spinal cord injury pain, temporomandibular disorder, myofascial pain, and syringomyelia.

According to specific embodiments, the neuropathic pain is central (originating in the brain or spinal cord) neuropathic pain.

According to specific embodiments, the central neuropathic pain is selected from the group consisting of: cerebral lesions that are predominantly thalamic, infarction, e.g. thalamic infarction or brain stem infarction, cerebral tumors or abscesses compressing the thalamus or brain stem, multiple sclerosis, brain operations, e.g. thalamotomy in cases of motoric disorders, spinal cord lesions, spinal cord injuries, spinal cord operations, e.g. anterolateral cordotomy, ischemic lesions, anterior spinal artery syndrome, Wallenberg's syndrome and syringomyelia.

According to specific embodiments, the pain is caused by spinal cord injury and/or spinal cord contusion.

According to specific embodiments, the pain is a head pain syndrome caused by central pain mechanisms.

According to specific embodiments, the neuropathic pain is peripheral (originating in the peripheral nervous system) neuropathic pain.

According to specific embodiments, the peripheral neuropathic pain is selected from the group consisting of peripheral denervation neuropathic pain, systemic diseases, e.g. diabetic neuropathy, drug-induced lesions, e.g. neuropathy due to chemotherapy, traumatic syndrome and entrapment syndrome, lesions in nerve roots and posterior ganglia, neuropathies after HIV infections, neuralgia after Herpes infections, nerve root avulsions, cranial nerve lesions, cranial neuralgias, e.g., trigeminal neuralgia, neuropathic cancer pain, phantom pain, compression of peripheral nerves, neuroplexus and nerve roots, paraneoplastic peripheral neuropathy and ganglionopathy, complications of cancer therapies, e.g. chemotherapy, irradiation, and surgical interventions, complex regional pain syndrome, type I lesions (previously known as sympathetic reflex dystrophy) and type II lesions (corresponding approximately to causalgia).

According to specific embodiments, the pain is a peripheral denervation neuropathic pain.

According to specific embodiments, the pain is a chemotherapy-induced neuropthic pain.

According to specific embodiments, the pain is not inflammatory pain.

According to specific embodiments, the pain is not associated with inflammation.

According to specific embodiments, the pain is not associated with inflammation in the vicinity of the origin of the pain.

According to specific embodiments, the pain is not associated with vascular inflammation.

As used herein, the term “target”, refers to importin α3 and to the polynucleotide or polypeptide expression product of a gene described by a Gene symbol in Tables 1-2.

As used herein the term “importin α3”, also known as Karyopherin Subunit Alpha 4, refers to the polynucleotide or polypeptide expression product of the KPNA4 gene (Gene ID: 3840). According to specific embodiments, the importin α3 refers to the human importin α3, such as provided in the following Accession Numbers: NM_002268, and NP_002259. According to specific embodiments, the importin α3 refers to the mouse importin α3, such as provided in the following Accession Numbers: NM_008467 and NP_032493.

According to specific embodiments, importin α3 activity is at least binding to c-Fos and acting as a chaperone transporting c-Fos into the nucleus (i.e. c-Fos nuclear transport).

As used herein the term “c-Fos”, refers to the polypeptide expression product of the FOS gene (Gene ID: 2353). According to specific embodiments, the c-Fos refers to the human c-Fos, such as provided in Accession Number: NP_005243. According to specific embodiments, the c-Fos refers to the mouse c-Fos, such as provided in Accession Number: NP_034364.

Hence, according to specific embodiments, the agent which inhibits activity of importin α3 binds an importin α3-C-Fos complex, interferes with formation of said importin α3-C-Fos complex or disintegrates said importin α3-C-Fos complex.

Assays for testing binding and complex formation are well known in the art and include, but not limited to immunoprecipitation, ELISA, flow cytometry, plasmon resonance, BIAcore assay and the like.

According to specific embodiments, the agent which inhibits activity of importin α3 inhibits importin α3 binding to the NLS sequence of c-Fos, as determined by e.g. immunoprecipitation, ELISA, flow cytometry or other known binding assays.

According to specific embodiments, importin α3 activity is at least binding to c-Jun and acting as a chaperone transporting c-Jun into the nucleus (i.e. c-Jun nuclear transport).

As used herein the term “c-Jun”, refers to the polypeptide expression product of the JUN gene (Gene ID: 3725). According to specific embodiments, the c-Jun refers to the human c-Jun, such as provided in Accession Number: NP_002219. According to specific embodiments, the c-Jun refers to the mouse c-Jun, such as provided in Accession Number: NP_034721.

Hence, according to specific embodiments, the agent which inhibits activity of importin α3 binds an importin α3-C-Jun complex, interferes with formation of said importin α3-C-Jun complex or disintegrates said importin α3-C-Jun complex. According to specific embodiments, the agent which inhibits activity of importin α3 inhibits importin α3 binding to the NLS sequence of c-Jun, as determined by e.g. immunoprecipitation, ELISA, flow cytometry or other known binding assays.

As mentioned, the agents disclosed herein are capable of modulating (inhibiting or enhancing, depending on the target) expression and/or activity of a target (e.g. importin α3, a target selected from the targets listed in Table 1 hereinabove, a target selected from the targets listed in Table 2 hereinvabove).

According to specific embodiments, the agent directly binds the target or a polynucleotide encoding same.

According to other specific embodiments, the agent indirectly binds the target by acting through an intermediary molecule, for example the agent binds to or modulates a molecule that in turn binds to or modulates the target.

As used herein, “modulating (i.e. inhibiting or enhancing) expression and/or activity” refers to a change (i.e. decrease or increase, respectively) of at least 5% in expression and/or biological function in the presence of the agent in comparison to same in the absence of the agent, as determined by e.g. PCR, ELISA, Western blot analysis, immunoprecipitation, flow cytometry, immuno-staining, kinase assays. As the agents of the present invention have an analgesic effect, the change can also be determined by pain models such as the noxious heat, chemically induced acute pain, and/or the spared nerve injury (SNI) model, which are further described in details in the Examples section which follows. According to a specific embodiment, the change is in at least 10%, 30%, 40% or even higher say, 50%, 60%, 70%, 80%, 90% or more than 100%.

According to specific embodiments, the change is at least 1.2 fold, at least 1.5 fold, at least 2 fold, at least 3 fold, at least 5 fold, at least 10 fold, or at least 20 fold as compared to same in the absence of the agent.

According to specific embodiments, the agent inhibits (down-regulates, decreases) expression and/or activity of the target.

Inhibiting expression and/or activity can be can be effected at the protein level (e.g., antibodies, small molecules, inhibitory peptides, enzymes that cleave the polypeptide, aptamers and the like) but may also be effected at the genomic (e.g. homologous recombination and site specific endonucleases) and/or the transcript level using a variety of molecules which interfere with transcription and/or translation (e.g., RNA silencing agents) of a target described herein.

Inhibition of expression may be either transient or permanent.

According to specific embodiments, inhibiting expression refers to the absence of mRNA and/or protein, as detected by RT-PCR or Western blot, respectively.

According to other specific embodiments inhibiting expression refers to a decrease in the level of mRNA and/or protein, as detected by RT-PCR or Western blot, respectively. The reduction may be by at least a 10%, at least 20%, at least 30%, at least 40%, at least 50%, at least 60%, at least 70%, at least 80%, at least 90%, at least 95% or at least 99% reduction.

Non-limiting examples of inhibiting agents are described in details hereinbelow.

Inhibition at the Polypeptide Level

According to specific embodiments, the inhibiting agent is an antibody.

According to specific embodiments the antibody is capable of specifically binding a target protein described herein.

According to specific embodiments, the antibody specifically binds at least one epitope of a target protein described herein.

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)2, Fv, scFv, dsFv, or single domain molecules such as VH and VL that are capable of binding to an epitope of an antigen. The antibody may be mono-specific (capable of recognizing one epitope or protein), bi-specific (capable of binding two epitopes or proteins) or multi-specific (capable of recognizing multiple epitopes or proteins).

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv Fv (scFv), a disulfide-stabilized Fv (dsFv), an Fab, an Fab′, and an F(ab′)2.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989), a compromise between Kabat and Chothia using Oxford Molecular's AbM antibody modeling software (now Accelrys®, see, Martin et al., 1989, Proc. Natl Acad Sci USA. 86:9268; and world wide web site www(dot)bioinf-org(dot)uk/abs), available complex crystal structures as defined by the contact definition (see MacCallum et al., J. Mol. Biol. 262:732-745, 1996), the “conformational definition” (see, e.g., Makabe et al., Journal of Biological Chemistry, 283:1156-1166, 2008) and IMGT [Lefranc M P, et al. (2003) IMGT unique numbering for immunoglobulin and T cell receptor variable domains and Ig superfamily V-like domains. Dev Comp Immunol 27: 55-77].

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:

(i) Fv, defined as a genetically engineered fragment consisting of the variable region of the light chain (VL) and the variable region of the heavy chain (VH) expressed as two chains;

(ii) single chain Fv (“scFv”), a genetically engineered single chain molecule including the variable region of the light chain and the variable region of the heavy chain, linked by a suitable polypeptide linker as a genetically fused single chain molecule.

(iii) disulfide-stabilized Fv (“dsFv”), a genetically engineered antibody including the variable region of the light chain and the variable region of the heavy chain, linked by a genetically engineered disulfide bond.

(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;

(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);

(vi) F(ab′) 2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds); and

(vii) Single domain antibodies or nanobodies are composed of a single VH or VL domains which exhibit sufficient affinity to the antigen.

The antibody may be monoclonal or polyclonal.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

It will be appreciated that for human therapy or diagnostics, humanized antibodies are preferably used. Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

As some of the targets described herein are localized intracellularly, the antibody or antibody fragment capable can be an intracellular antibody (also known as “intrabodies”). Intracellular antibodies are essentially SCA to which intracellular localization signals have been added (e.g., ER, mitochondrial, nuclear, cytoplasmic). This technology has been successfully applied in the art (for review, see Richardson and Marasco, 1995, TIBTECH vol. 13). Intrabodies have been shown to virtually eliminate the expression of otherwise abundant cell surface receptors and to inhibit a protein function within a cell (See, for example, Richardson et al., 1995, Proc. Natl. Acad. Sci. USA 92: 3137-3141; Deshane et al., 1994, Gene Ther. 1: 332-337; Marasco et al., 1998 Human Gene Ther 9: 1627-42; Shaheen et al., 1996 J. Virol. 70: 3392-400; Werge, T. M. et al., 1990, FEBS Letters 274:193-198; Carlson, J. R. 1993 Proc. Natl. Acad. Sci. USA 90:7427-7428; Biocca, S. et al., 1994, Bio/Technology 12: 396-399; Chen, S-Y. et al., 1994, Human Gene Therapy 5:595-601; Duan, L et al., 1994, Proc. Natl. Acad. Sci. USA 91:5075-5079; Chen, S-Y. et al., 1994, Proc. Natl. Acad. Sci. USA 91:5932-5936; Beerli, R. R. et al., 1994, J. Biol. Chem. 269:23931-23936; Mhashilkar, A. M. et al., 1995, EMBO J. 14:1542-1551; PCT Publication No. WO 94/02610 by Marasco et al.; and PCT Publication No. WO 95/03832 by Duan et al.).

To prepare an intracellular antibody expression vector, the cDNA encoding the antibody light and heavy chains specific for the target protein of interest are isolated, typically from a hybridoma that secretes a monoclonal antibody specific for the marker. Hybridomas secreting anti-marker monoclonal antibodies, or recombinant monoclonal antibodies, can be prepared using methods known in the art. Once a monoclonal antibody specific for the marker protein is identified (e.g., either a hybridoma-derived monoclonal antibody or a recombinant antibody from a combinatorial library), DNAs encoding the light and heavy chains of the monoclonal antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process and the nucleotide sequences of antibody light and heavy chain genes are determined. For example, many such sequences are disclosed in Kabat, E. A., et al. (1991) Sequences of Proteins of Immunological Interest, Fifth Edition, U.S. Department of Health and Human Services, NIH Publication No. 91-3242 and in the “Vbase” human germline sequence database. Once obtained, the antibody light and heavy chain sequences are cloned into a recombinant expression vector using standard methods.

For cytoplasmic expression of the light and heavy chains, the nucleotide sequences encoding the hydrophobic leaders of the light and heavy chains are removed. An intracellular antibody expression vector can encode an intracellular antibody in one of several different forms. For example, in one embodiment, the vector encodes full-length antibody light and heavy chains such that a full-length antibody is expressed intracellularly. In another embodiment, the vector encodes a full-length light chain but only the VH/CH1 region of the heavy chain such that a Fab fragment is expressed intracellularly. In another embodiment, the vector encodes a single chain antibody (scFv) wherein the variable regions of the light and heavy chains are linked by a flexible peptide linker [e.g., (Gly₄Ser)₃ and expressed as a single chain molecule. To inhibit marker activity in a cell, the expression vector encoding the intracellular antibody is introduced into the cell by standard transfection methods, as discussed hereinbefore.

Once antibodies are obtained, they may be tested for activity, for example via ELISA.

Another inhibiting agent which can be used along with some embodiments of the invention is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

Another inhibiting agent would be any molecule which interferes with the target protein activity (e.g., catalytic or interaction) by binding the target protein or intermediate thereof and/or cleaving the target protein. Such molecules can be a small molecule, antagonists, or inhibitory peptide.

Another inhibiting agent which can be used along with some embodiments of the invention is a molecule which prevents target activation or substrate binding.

According to a specific embodiment, the inhibiting agent is a small molecule.

According to a specific embodiment, the inhibiting agent is a peptide molecule (i.e. an inhibitory peptide, also referred to as “dominant negative”).

According to specific embodiments, the inhibitory peptide is devoid of a catalytic activity (e.g. in the case of a peptide comprising an amino acid sequence of c-Fos or c-Jun, as further described hereinbelow, the peptide does not have a transcription factor activity).

According to specific embodiments, the peptide is less than 50 amino acids in length.

According to specific embodiments, the peptide is less than 45 amino acids in length.

According to specific embodiments, the peptide is less than 30 amino acids in length.

According to specific embodiments, the peptide is 20-50 amino acids in length.

A non-limiting example of an importin α3 inhibitory peptide can be an amino acid sequence of c-FOS.

As used herein, the term “amino acid sequence of C-Fos” refers to a portion of C-Fos or a functional homologue (naturally occurring or synthetically/recombinantly produced) thereof, which maintains the ability to bind importin α3.

According to specific embodiments, the inhibitory peptide does not comprise the full length native c-Fos.

According to a specific embodiment, the inhibitory peptide is a portion of C-Fos which comprises the nuclear localization sequence (NLS) of C-Fos. Typically, such an amino acid sequence comprises amino acids residues 131-145 corresponding to Accession Number: NP_005243.

Thus, according to specific embodiments, the inhibitory peptide comprises SEQ ID NO: 20.

Another non-limiting example of an importin α3 inhibitory peptide can be an amino acid sequence of c-Jun.

As used herein, the term “amino acid sequence of C-Jun” refers to a portion of C-Jun or a functional homologue (naturally occurring or synthetically/recombinantly produced) thereof, which maintains the ability to bind importin α3.

According to specific embodiments, the inhibitory peptide does not comprise the full length native c-Fos.

According to a specific embodiment, the inhibitory peptide is a portion of C-Jun which comprises the nuclear localization sequence (NLS) of C-Jun. Typically, such an amino acid comprises amino acids residues 252-293 corresponding to Accession Number: NP_002219.

Thus, according to specific embodiments, the inhibitory peptide comprises SEQ ID NO: 21.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C.A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH3)—CO—), ester bonds (—C(═O)—O—), ketomethylene bonds (—CO—CH2-), sulfinylmethylene bonds (—S(═O)—CH2-), α-aza bonds (—NH—N(R)—CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2—CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 7 and 7 below list naturally occurring amino acids (Table 6), and non-conventional or modified amino acids (e.g., synthetic, Table 7) which can be used with some embodiments of the invention.

TABLE 6

| Amino Acid | Three-Letter Abbreviation | One-letter Symbol |
|---|---|---|
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 7

| Non-conventional amino | | | |
|---|---|---|---|
| acid | Code | Non-conventional amino acid | Code |
| ornithine | Orn | hydroxyproline | Hyp |
| α-aminobutyric acid | Abu | aminonorbornyl- | Norb |
| D-alanine | Dala | carboxylate | |
| D-arginine | Darg | aminocyclopropane- | Cpro |
| D-asparagine | Dasn | carboxylate | |
| D-aspartic acid | Dasp | N-(3-guanidinopropyl)glycine | Narg |
| D-cysteine | Dcys | N-(carbamylmethyl)glycine | Nasn |
| D-glutamine | Dgln | N-(carboxymethyl)glycine | Nasp |
| D-glutamic acid | Dglu | N-(thiomethyl)glycine | Ncys |
| D-histidine | Dhis | N-(2-carbamylethyl)glycine | Ngln |
| D-isoleucine | Dile | N-(2-carboxyethyl)glycine | Nglu |
| D-leucine | Dleu | N-(imidazolylethyl)glycine | Nhis |
| D-lysine | Dlys | N-(1-methylpropyl)glycine | Nile |
| D-methionine | Dmet | N-(2-methylpropyl)glycine | Nleu |
| D-ornithine | Dorn | N-(4-aminobutyl)glycine | Nlys |
| D-phenylalanine | Dphe | N-(2-methylthioethyl)glycine | Nmet |
| D-proline | Dpro | N-(3-aminopropyl)glycine | Norn |
| D-serine | Dser | N-benzylglycine | Nphe |
| D-threonine | Dthr | N-(hydroxymethyl)glycine | Nser |
| D-tryptophan | Dtrp | N-(1-hydroxyethyl)glycine | Nthr |
| D-tyrosine | Dtyr | N-(3-indolylethyl) glycine | Nhtrp |
| D-valine | Dval | N-(p-hydroxyphenyl)glycine | Ntyr |
| D-N-methylalanine | Dnmala | N-(1-methylethyl)glycine | Nval |
| D-N-methylarginine | Dnmarg | N-methylglycine | Nmgly |
| D-N-methylasparagine | Dnmasn | L-N-methylalanine | Nmala |
| D-N-methylasparatate | Dnmasp | L-N-methylarginine | Nmarg |
| D-N-methylcysteine | Dnmcys | L-N-methylasparagine | Nmasn |
| D-N-methylglutamine | Dnmgln | L-N-methylaspartic acid | Nmasp |
| D-N-methylglutamate | Dnmglu | L-N-methylcysteine | Nmcys |
| D-N-methylhistidine | Dnmhis | L-N-methylglutamine | Nmgln |
| D-N-methylisoleucine | Dnmile | L-N-methylglutamic acid | Nmglu |
| D-N-methylleucine | Dnmleu | L-N-methylhistidine | Nmhis |
| D-N-methyllysine | Dnmlys | L-N-methylisolleucine | Nmile |
| D-N-methylmethionine | Dnmmet | L-N-methylleucine | Nmleu |
| D-N-methylornithine | Dnmorn | L-N-methyllysine | Nmlys |
| D-N-methylphenylalanine | Dnmphe | L-N-methylmethionine | Nmmet |
| D-N-methylproline | Dnmpro | L-N-methylornithine | Nmorn |
| D-N-methylserine | Dnmser | L-N-methylphenylalanine | Nmphe |
| D-N-methylthreonine | Dnmthr | L-N-methylproline | Nmpro |
| D-N-methyltryptophan | Dnmtrp | L-N-methylserine | Nmser |
| D-N-methyltyrosine | Dnmtyr | L-N-methylthreonine | Nmthr |
| D-N-methylvaline | Dnmval | L-N-methyltryptophan | Nmtrp |
| L-norleucine | Nle | L-N-methyltyrosine | Nmtyr |
| L-norvaline | Nva | L-N-methylvaline | Nmval |
| L-ethylglycine | Etg | L-N-methylnorleucine | Nmnle |
| L-t-butylglycine | Thug | L-N-methylnorvaline | Nmnva |
| L-homophenylalanine | Hphe | L-N-methyl-ethylglycine | Nmetg |
| α-naphthylalanine | Anap | L-N-methyl-t-butylglycine | Nmtbug |
| penicillamine | Pen | L-N-methyl-homophenylalanine | Nmhphe |
| γ-aminobutyric acid | Gabu | N-methyl-α-naphthylalanine | Nmanap |
| cyclohexylalanine | Chexa | N-methylpenicillamine | Nmpen |
| cyclopentylalanine | Cpen | N-methyl-γ-aminobutyrate | Nmgabu |
| α-amino-α-methylbutyrate | Aabu | N-methyl-cyclohexylalanine | Nmchexa |
| α-aminoisobutyric acid | Aib | N-methyl-cyclopentylalanine | Nmcpen |
| D-α-methylarginine | Dmarg | N-methyl-α-amino-α- | Nmaabu |
| D-α-methylasparagine | Dmasn | methylbutyrate | |
| D-α-methylaspartate | Dmasp | N-methyl-α-aminoisobutyrate | Nmaib |
| D-α-methylcysteine | Dmcys | L-α-methylarginine | Marg |
| D-α-methylglutamine | Dmgln | L-α-methylasparagine | Masn |
| D-α-methyl glutamic acid | Dmglu | L-α-methylaspartate | Masp |
| D-α-methylhistidine | Dmhis | L-α-methylcysteine | Mcys |
| D-α-methylisoleucine | Dmile | L-α-methylglutamine | Mgln |
| D-α-methylleucine | Dmleu | L-α-methylglutamate | Mglu |
| D-α-methyllysine | Dmlys | L-α-methylhistidine | Mhis |
| D-α-methylmethionine | Dmmet | L-α-methylisoleucine | Mile |
| D-α-methylornithine | Dmorn | L-α-methylleucine | Mleu |
| D-α-methylphenylalanine | Dmphe | L-α-methyllysine | Mlys |
| D-α-methylproline | Dmpro | L-α-methylmethionine | Mmet |
| D-α-methylserine | Dmser | L-α-methylornithine | Morn |
| D-α-methylthreonine | Dmthr | L-α-methylphenylalanine | Mphe |
| D-α-methyltryptophan | Dmtrp | L-α-methylproline | Mpro |
| D-α-methyltyrosine | Dmtyr | L-α-methylserine | Mser |
| D-α-methylvaline | Dmval | L-α-methylthreonine | Mthr |
| N-cyclobutylglycine | Ncbut | L-α-methyltryptophan | Mtrp |
| N-cycloheptylglycine | Nchep | L-α-methyltyrosine | Mtyr |
| N-cyclohexylglycine | Nchex | L-α-methylvaline | Mval |
| N-cyclodecylglycine | Ncdec | L-α-methylnorvaline | Mnva |
| N-cyclododecylglycine | Ncdod | L-α-methylethylglycine | Metg |
| N-cyclooctylglycine | Ncoct | L-α-methyl-t-butylglycine | Mtbug |
| N-cyclopropylglycine | Ncpro | L-α-methyl-homophenylalanine | Mhphe |
| N-cycloundecylglycine | Ncund | α-methyl-α-naphthylalanine | Manap |
| N-(2-aminoethyl)glycine | Naeg | α-methylpenicillamine | Mpen |
| N-(2,2-diphenylethyl)glycine | Nbhm | α-methyl-γ-aminobutyrate | Mgabu |
| N-(3,3- | Nbhe | α-methyl-cyclohexylalanine | Mchexa |
| diphenylpropyl)glycine | | α-methyl-cyclopentylalanine | Mcpen |
| 1-carboxy-1-(2,2-diphenyl | Nmbc | N-(N-(2,2-diphenylethyl) | Nnbhm |
| ethylamino)cyclopropane | | carbamylmethyl-glycine | |
| phosphoserine | pSer | N-(N-(3,3-diphenylpropyl) | Nnbhe |
| phosphotyrosine | pTyr | carbamylmethyl-glycine | |
| 2-aminoadipic acid | | 1,2,3,4-tetrahydroisoquinoline- | Tic |
| | | 3-carboxylic acid | |
| | | phosphothreonine | pThr |
| | | O-methyl-tyrosine | |
| | | hydroxylysine | |

Since the present peptides are preferably utilized in therapeutics or diagnostics which require the peptides to be in soluble form, the peptides of some embodiments of the invention preferably include one or more non-natural or natural polar amino acids, including but not limited to serine and threonine which are capable of increasing peptide solubility due to their hydroxyl-containing side chain.

The peptides of some embodiments of the invention are preferably utilized in a linear form, although it will be appreciated that in cases where cyclicization does not severely interfere with peptide characteristics, cyclic forms of the peptide can also be utilized.

According to specific embodiments, the peptide is attached to a cell-penetrating peptide.

As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of some embodiments of the invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of some embodiments of the invention preferably include, but are not limited to, penetratin, transportan, pIsl, TAT(48-60), pVEC, MTS, and MAP.

The peptides of some embodiments of the invention may be synthesized by any techniques that are known to those skilled in the art of peptide synthesis such as, but not limited to, solid phase and recombinant techniques as further described in details hereinbelow. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide compounds of some embodiments of the invention involves solid phase peptide synthesis.

Large scale peptide synthesis is described by Andersson Biopolymers 2000; 55(3):227-50.

It will be appreciated that a non-functional analogue of at least a catalytic or binding portion of the target can be also used as an inhibiting agent.

Inhibition at the Nucleic Acid Level

Inhibition at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

Thus, inhibition can be achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g., importin α3) and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Methods of introducing RNA silencing agents are known in the art and include naked RNA as well as incorporation into vectors e.g. viral vactors e.g. AAV, vectors that enter the blood brain barrier (BBB).

According to a specific embodiments, the RNA silencing agent is provided as a naked RNA (not part of an expression vector).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to inhibit protein expression from mRNA.

According to one embodiment dsRNA longer than 30 bp are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

According to some embodiments of the invention, dsRNA is provided in cells where the interferon pathway is not activated, see for example Billy et al., PNAS 2001, Vol 98, pages 14428-14433; and Diallo et al, Oligonucleotides, Oct. 1, 2003, 13(5): 381-392. doi: 10.1089/154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www(dot)ambion(dot)com/techlib/tn/91/912(dot)html).

Second, potential target sites are compared to an appropriate genomic database (e.g., human, mouse, rat etc.) using any sequence alignment software, such as the BLAST software available from the NCBI server (www(dot)ncbi(dot)nlm(dot)nih(dot)gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

It will be appreciated that, and as mentioned hereinabove, the RNA silencing agent of some embodiments of the invention need not be limited to those molecules containing only RNA, but further encompasses chemically-modified nucleotides and non-nucleotides.

Non-limiting examples of importin α3 siRNA that can be used with specific embodiments of the invention can be commercially obtained from e.g. Dharmacon [e.g. ON-TARGETplus siRNAs for mouse importins: importin α3, L-058423-01 (Huenninger et al., 2010)] or ORIGENE (e.g. CAT #SR302597 KPNA4 Human siRNA Oligo Duplex).

Non-limiting examples of importin α3 shRNA that can be used with specific embodiments of the invention include SEQ ID NO: 8 or KPNA4importin alpha3 Human shRN lentiviral particles commercialy available from ORIGENE (CAT #TL311850V).

miRNA and miRNA mimics—According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Below is a brief description of the mechanism of miRNA activity.

Genes coding for miRNAs are transcribed leading to production of an miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al. (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to down-regulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” or “miRNA mimic” refers to synthetic non-coding RNAs that are capable of entering the RNAi pathway and regulating gene expression. miRNA mimics imitate the function of endogenous miRNAs and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

Preparation of miRNAs mimics can be effected by any method known in the art such as chemical synthesis or recombinant methods.

It will be appreciated from the description provided herein above that contacting cells with a miRNA may be effected by transfecting the cells with e.g. the mature double stranded miRNA, the pre-miRNA or the pri-miRNA.

The pre-miRNA sequence may comprise from 45-90, 60-80 or 60-70 nucleotides.

The pri-miRNA sequence may comprise from 45-30,000, 50-25,000, 100-20,000, 1,000-1,500 or 80-100 nucleotides.

A non-limiting example of importin α3 miRNA that can be used with specific embodiments of the invention include MiR-181b, which was found to specifically down-regulate importin-α3 expression, thereby blocking NF-κB import and signalling in epithelial cells (Sun et al., 2012, J. Clin. Invest. 122:1973-1990 [PubMed: 22622040]; Sun et al., 2014, Circ. Res. 114:32-40. [PubMed: 24084690]).

Antisense—Antisense is a single stranded RNA designed to prevent or inhibit expression of a gene by specifically hybridizing to its mRNA. Inhibition can be effected using an antisense polynucleotide capable of specifically hybridizing with an mRNA transcript encoding the target (e.g. importin α3).

Design of antisense molecules which can be used to efficiently down-regulate a target must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Jääskeläinen et al. Cell Mol Biol Lett. (2002) 7(2):236-7; Gait, Cell Mol Life Sci. (2003) 60(5):844-53; Martino et al. J Biomed Biotechnol. (2009) 2009:410260; Grijalvo et al. Expert Opin Ther Pat. (2014) 24(7):801-19; Falzarano et al, Nucleic Acid Ther. (2014) 24(1):87-100; Shilakari et al. Biomed Res Int. (2014) 2014: 526391; Prakash et al. Nucleic Acids Res. (2014) 42(13):8796-807 and Asseline et al. J Gene Med. (2014) 16(7-8):157-65]

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)]. Such algorithms have been successfully used to implement an antisense approach in cells.

In addition, several approaches for designing and predicting efficiency of specific oligonucleotides using an in vitro system were also published (Matveeva et al., Nature Biotechnology 16: 1374-1375 (1998)].

Thus, the generation of highly accurate antisense design algorithms and a wide variety of oligonucleotide delivery systems, enable an ordinarily skilled artisan to design and implement antisense approaches suitable for down-regulating expression of known sequences without having to resort to undue trial and error experimentation.

A non-limiting example of importin α3 antisense that can be used according to some embodiments of the invention include SEQ ID NO: 22.

Nucleic acid agents can also operate at the DNA level as summarized infra.

Inhibition can also be achieved by inactivating the gene (e.g., KPNA4) via introducing targeted mutations involving loss-of function alterations (e.g. point mutations, deletions and insertions) in the gene structure.

As used herein, the phrase “loss-of-function alterations” refers to any mutation in the DNA sequence of a gene which results in down-regulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a read through protein) which affects the secondary or tertiary structure of the protein and results in a non-functional protein, devoid of the enzymatic activity of the non-mutated polypeptide; a read through mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments loss-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments loss-of-function alteration of a gene comprises both alleles of the gene. In such instances the e.g. KPNA4 may be in a homozygous form or in a heterozygous form.

Methods of introducing nucleic acid alterations to a gene of interest are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8, 129,134; 8,133,697; 8,143,015; 8,143,016; 8, 148,098; or 8, 163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fokl domains heterodimerize to create a double-stranded break. Repair of these double-stranded breaks through the non-homologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

CRISPR-Cas system—Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH—, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], To12 [Kawakami et al. PNAS (2000) 97 (21): 11403-11408] or Frog Prince [Miskey et al. Nucleic Acids Res. Dec. 1, (2003) 31(23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas To12 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

It will be appreciated that the agent can be a mutagen that causes random mutations and the cells exhibiting down-regulation of the expression level and/or activity of the target may be selected.

The mutagens may be, but are not limited to, genetic, chemical or radiation agents. For example, the mutagen may be ionizing radiation, such as, but not limited to, ultraviolet light, gamma rays or alpha particles. Other mutagens may include, but not be limited to, base analogs, which can cause copying errors; deaminating agents, such as nitrous acid; intercalating agents, such as ethidium bromide; alkylating agents, such as bromouracil; transposons; natural and synthetic alkaloids; bromine and derivatives thereof; sodium azide; psoralen (for example, combined with ultraviolet radiation). The mutagen may be a chemical mutagen such as, but not limited to, ICR191, 1,2,7,8-diepoxy-octane (DEO), 5-azaC, N-methyl-N-nitrosoguanidine (MNNG) or ethyl methane sulfonate (EMS).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosytransferase (ARPT).

According to specific embodiments, the agent enhances (up-regulates, increases) expression and/or activity of the target.

Enhancing expression and/or activity can be effected at the protein level (e.g., antibodies, small molecules, peptides and the like) but may also be effected at the genomic level (e.g., activation of transcription via promoters, enhancers, regulatory elements) and/or the transcript level using a variety of molecules which promote transcription and/or translation (e.g., correct splicing, polyadenylation, activation of translation) of a target described herein.

Non-limiting examples of agents that can function as enhancing agents are described in details hereinbelow.

Enhancement at the Polypeptide Level

According to specific embodiments, the agonist is the naturally occurring activator or a functional derivative or variant thereof which retain the ability to specifically bind to the target protein.

It will be appreciated that a functional analogue of at least a catalytic or binding portion of a target protein can be also used as an enhancing agent. Thus, according to specific embodiments, the agent is an exogenous polypeptide including at least a functional portion (e.g. catalytic or interaction) of the target protein.

According to specific embodiments, the agonist is an antibody.

According to specific embodiments the antibody is capable of specifically binding a target protein described herein.

A detailed description on antibodies that can be used according to specific embodiments of the present invention is provided hereinabove.

Another enhancing agent would be a molecule which promotes and/or increases the function (e.g. catalytic or interaction) of the target protein by binding to the target or an intermediate thereof. Such molecules can be, but are not limited to, small molecules, peptides and aptamers, wherein each possibility is a separate embodiment of the invention.

According to specific embodiments, the agent is a peptide.

According to specific embodiments, the agent is a small molecule.

Enhancement at the Nucleic Acid Level

The enhancing agent can also be a molecule which is capable of increasing the transcription and/or translation of an endogenous DNA or mRNA encoding the target protein.

Another enhancing agent may be an exogenous polynucleotide (DNA or RNA) sequence designed and constructed to express at least a functional portion of the target protein. The coding sequences information for the targets described herein is available from several databases including the GenBank database available through www(dot)ncbi (dot)nlm (dot)nih(dot)gov/, and is further described hereinabove.

To express an exogenous protein in mammalian cells, a polynucleotide sequence encoding a specific protein or a homologue thereof which exhibit the desired activity is preferably ligated into a nucleic acid construct suitable for mammalian cell expression. Such a nucleic acid construct includes a promoter sequence for directing transcription of the polynucleotide sequence in the cell in a constitutive [e.g. cytomegalovirus (CMV) and Rous sarcoma virus (RSV)] or inducible (e.g. the tetracycline-inducible promoter) manner. According to specific embodiments, the promoter utilized by the nucleic acid construct of some embodiments of the invention is active in a specific cell population.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, a typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof. The construct may also include an enhancer element which can stimulate transcription up to 1,000 fold from linked homologous or heterologous promoters. The vector may or may not include a eukaryotic replicon.

The nucleic acid construct of some embodiments of the invention can also include a signal sequence for secretion of the peptide from a host cell in which it is placed. Preferably the signal sequence for this purpose is a mammalian signal sequence or the signal sequence of the polypeptide variants of some embodiments of the invention.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

Other than containing the necessary elements for the transcription and translation of the inserted coding sequence, the expression construct of some embodiments of the invention can also include sequences engineered to enhance stability, production, or yield of the expressed peptide.

It will be appreciated that the individual elements comprised in the expression vector can be arranged in a variety of configurations.

The type of vector used by some embodiments of the invention will depend on the cell type transformed. The ability to select suitable vectors according to the cell type transformed is well within the capabilities of the ordinary skilled artisan and as such no general description of selection consideration is provided herein.

Recombinant viral vectors are useful for in vivo expression of a protein since they offer advantages such as lateral infection and targeting specificity. Viral vectors can also be produced that are unable to spread laterally.

Various methods can be used to introduce the expression vector of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986]. Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. Other vectors can be used that are non-viral, such as cationic lipids, polylysine, and dendrimers.

Agents which can be implemented in the present teachings can be identified according to the following aspect.

According to an aspect of the present invention, there is provided a method of identifying a compound for treating pain, the method comprising determining a transcriptional signature of a neuronal cell following treatment with a test compound and comparing said transcriptional signature of said neuronal cell following said treatment to a transcriptional signature of an importin alpha3 deficient neuronal cell, wherein a similar transcriptional signature indicates efficacy of said test compound for treating pain.

Determining a transcriptome signature can be effect by any method known in the art, such as, but not limited to RNA-seq.

According to specific embodiments, determining is effected in-vitro or ex-vivo.

According to specific embodiments, the importin alpha3 deficient neuronal cell is an importin alpha3 null cell.

According to specific embodiments, the neuronal cell is a sensory neuron.

According to specific embodiments, the neuronal cell is a dorsal root ganglion cell.

Typically, the agents identified by the method described herein are analgesic agents.

Thus, according to specific embodiments, the screening method further comprising providing the test agent and testing an analgesic activity of same.

Typically, the agents identified by the method described herein are suitable for treating pain.

Thus, according to specific embodiments, the screening method further comprising treating pain in a subject in need thereof with the test compound when efficacy of the test compound for treating pain is indicated.

The agents and the compounds of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent or compound accountable for the biological effect.

According to specific embodiments, the agent is the active agent in the formulation.

According to specific embodiments, the agent is the sole active agent in the formulation.

According to specific embodiments, the compound is the active agent in the formulation.

According to specific embodiments, the compound is the sole active agent in the formulation.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

According to specific embodiments, the agent is provided by intrathecal injection.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of pain (e.g., nociceptive pain, neuropathic pain) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Examples of animal models that can be used to asses an analgesic effect include, but are not limited to, animal models of nociceptive pain e.g. a response to noxious heat and chemical (e.g. capsaicin) induced acute pain such as described in the Examples section which follows; and animal models of neuropathic pain e.g. the Chung spinal segmental nerve, the Bennett chronic constriction injury, the Seltzer partial sciatic nerve injury and the spared nerve injury models.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

It will be appreciated that the agents and compositions comprising same of the instant invention can be co-administered (sequentially and/or simultaneously) with other analgesic or with other therapeutics. Thus, for example other analgesics that can be administered in combination with the agents or compounds of some embodiments of the present invention include, but not limited to, acetaminophen, NSAIDs (e.g. ibuprofen, naproxen), Corticosteroids, Opioids, Antidepressants, Lidocaine patches.

According to specific embodiments, the agent or the compound is not administered in combination with another analgesic.

According to specific embodiments, the agent or the compound is not conjugated to another therapeutic moiety.

According to specific embodiments, the agent or the compound is not conjugated to another analgesic.

According to specific embodiments, the agent or the compound is not conjugated to a targeting moiety.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Mice—All animal experiments were reviewed and approved by the Institutional Animal Care and Use Committee (IACUC) of the Weizmann Institute of Science. Importin α single gene knockouts for importin al, α3, α4, α5 and α7 were generated by conventional gene deletion strategies^{7,10-12}. C57BL/6 mice were from Envigo Ltd (Israel). All mouse strains used were bred and kept at 24° C. in a humidity-controlled room under a 12 hours light-dark cycle with free access to food and water. Experiments were carried out on animals 2-5 months old.

Pain Models—Responses to noxious heat were assessed by applying a metal probe heated to 58° C. to a forelimb paw, while holding the animal. Paw withdrawal latency was timed, typically ranging between 2-4 seconds in wild-type animals. If the paw was not withdrawn within 20 seconds the assay was terminated. The test was repeated three times for each animal, with at least 20 minute intervals between repeats. Heat sensitivity was also assessed using the hot plate test [L. Urien et al., Sci Rep 7, 43493 (2017)]. To this end, mice were placed individually in a 20 cm high Plexiglas box on a metal surface set at 52, 55 or 58° C., and the latency to initiate a nociceptive response (licking, hind paws shaking, jumping) was monitored by videotape. Mice were removed from the plate immediately after a nociceptive response.

The behavioral response to cold stimulation was tested using the acetone evaporation test [J. P. Golden et al., J Neurosci 30, 3983-3994 (2010)]. In brief, acetone (100%, 70 μl) was applied twice onto the plantar surface of the hind paw using a micropipette with an interval of 20 minutes between each application. Animals were then videotaped for one minute and the latency to initiate hind paw licking was measured.

Acute pain related behaviours induced by plantar injection of capsaicin (50 μg/kg) into the hind paw was assessed as previously described [Nakamori et al. J Nat Med 71, 105 (2017)]. Mice were placed in a transparent cylinder and video recorded for three minutes following injection. Paw licking time and latency were measured in seconds.

Chronic neuropathic pain was assessed using the spared nerve injury (SNI) model¹³. Mice were anaesthetized with Ketamine/Xylazine (10 mg/kg body weight, intraperitoneal). The skin on the lateral surface of the thigh was incised and a section was made directly through the biceps femoris muscle in order to expose the sciatic nerve and its three terminal branches: the sural, common peroneal and tibial nerves. The SNI procedure comprised an axotomy and ligation of the tibial and common peroneal nerves leaving the sural nerve intact. The peroneal and the tibial nerves were tight-ligated and sectioned distal to the ligation. The lesion resulted in a marked hypersensitivity in the lateral area of the paw innervated by the spared sural nerve. Following, mice were evaluated over a period of three months.

Behavioral Tests—All assays were performed during the “dark” active phase of the diurnal cycle under dim illumination (˜10 1×) unless otherwise stated. The ventilation system in the test rooms provided a ˜65 dB white noise background. Every daily testing session started with one hour habituation to the test room. A recovery period of at least one day was provided between the different behavioral assays. Animals were marked with transient dye labels on the tails to avoid unnecessary stress and to enable blinded testing.

Von Frey tests of sensitivity to mechanical stimuli were conducted as previously described [Marvaldi et al. Dev Neurobiol 75, 217 (2015)]. Briefly, mice were placed in acrylic chambers suspended above a wire mesh grid and allowed to habituate to the testing apparatus for one hour prior to experiments. When the mouse was calm, the von Frey filaments were pressed against the plantar surface of the paw until the filament buckled and held for a maximum of 3 seconds. A positive response was noted if the paw was sharply withdrawn on application of the filament. Testing began with filament target force 13.7 milliNewtons and progressed according to an up-down method. 2 gram Von Frey filaments were used to assess sensitivity to noxious mechanical stimulation, scoring mice responses as follows: 0, no response; 1, visible signs of discomfort without leg withdrawal; 2, withdrawal of the leg.

CatWalk gait analysis and training was carried out as previously described [Perry et al. Neuron 75, 294 (2012)]. Motivation was achieved by a combination of food restriction during the initial training and placing of palatable reward at runway ends. The test was repeated three times for each mouse. Data were collected and analyzed using the Catwalk Ethovision XT11 software (Noldus Information Technology, The Netherlands). The analyzed indices are reported for each animal as print area and print width.

Rotarod experiments to assess integrity of balance and coordination [Crawley. Neuron 57, 809 (2008)] was carried out using a ROTOR-ROD™ system (83×91×61-SD Instruments, San Diego). Mice were subjected to three trials with 20 minutes inter-trial intervals over three consecutive days, at three weeks and five weeks after AAV9 injection, calculating the daily average each time. Rotarod acceleration was set to 20 rpm in 240 seconds. Latency to fall (sec) was recorded and the average of the three or six consecutive trials was used as an index of motor coordination and balance.

The wire hanging test was used to examine motor neuromuscular impairment and motor coordination, as previously described [Rafael et al. Mamm Genome 11, 725 (2000)]. Forepaws of the tested mouse were allowed to grasp and hold the animal suspended on an elevated metal wire (diameter 2 mm, length 90 cm) 80 cm above a water-filled tank. Traction was determined as the ability not to drop from the wire and to remain stable and hanging. The time (sec) until the mouse completely released its grip was recorded.

The pole test served to assess basal ganglia-related movement functions, as described Matsuura et al. J Neurosci Methods 73, 45 (1997)]. Briefly, mice were placed head-up on top of a 50 cm-long horizontal pole (1 cm in diameter). The base of the pole was placed in the home cage. When the pole is flipped downward, animals orient themselves (turn) and descend the length of the pole back into their home cage. Mice received two days of training on the horizontal pole, consisting of five trials for each session. On the test day, animals received five trials, and time to orient downward T_{Turn} was recorded. If a mouse was not able to turn or fell, a cut-off value of 120 sec was assigned.

Motility and anxiety-like behaviors were assayed in the open field (OF) as previously described⁸. Mouse activity was tracked and recorded using VideoMot2 (TSE System, Germany). OF was performed under 120 1× for the assessment of anxiety-related behaviors. The OF raw data were further analyzed with COLORcation [Dagan et al. J Neurosci Methods 270, 9 (2016)].

Pain coping behavior was monitored by quantification of paw licking of the injured limb, recording mouse activity over a period of 10 minutes inside a transparent enclosure (15×29×12 cm) containing a ˜1 cm layer of cage litter. Recording was conducted using a high-resolution GigE camera directly connected to Noldus Media Recorder software (Noldus, Wageningen, the Netherlands), collecting both top and lateral views in the same video by positioning a 45° angle mirror above the cage. Spontaneous licking of the SNI-injured paw was quantified at one week after SNI and in both AAV-PHP.S-shCtrl and AAV-PHP.S-shα3-injected mice 12 weeks after the injury. Recordings were analyzed off-line in a blinded manner to determine accumulative paw licking duration during the recording period.

Histology—Lumbar sections of the spinal cord including Dorsal root ganglia (DRGs) were pre-fixed for 6 minutes before dissection of spinal cord and associated DRGs. Lumbar DRG (L4, unless otherwise indicated) and/or spinal cord were then fixed for six hours in 4% PFA in PBS, washed in PBS and equilibrated in 20% w/v sucrose in PBS prior to serial cryo-sectioning at a thickness of 10-20 μm. Following, one set for each DRG was processed for immunostaining. Briefly, sections were rehydrated in PBS, blocked and permeabilized with 15% Donkey Serum, 5-10% BSA, 0.3% Triton X-100 in PBS for 1-3 hours and incubated overnight at 4° C. with Mouse anti-βIII tubulin (TuJ1, abcam, ab18207), Rabbit anti-cFos (Millipore, Ab5, 4-17), Mouse anti-cFOS (Millipore Ab5 4-17, IF 1:500), Mouse anti-Jun (BD transduction Laboratories, IF 1:1000), Rabbit TRPV1 (Alomone ACC-030, 1:500), Rabbit anti-GFP (abcam, ab6556, IF & WB1:500), Goat anti-CGRP (AbD SEROTEC, 1720-9007), MBP (Abcam ab7349, IF 1:500) or Rabbit anti-importin α3 (1:2000) antibodies. On the following day, sections were washed three times in PBS prior to incubation for 2 hours with different combinations of donkey anti-chicken/rabbit/mouse secondary antibodies (Alexa Fluor 647, 594, 488; Jackson Immunoresearch, 1:1000). Following, coverslips or slides were washed and mounted with Flouromount-G™.

Image processing—Images were acquired on a confocal laser-scanning microscope (Olympus FV1000, 60× oil-immersion objective Olympus UPLSAPO-NA 1.35) using Fluoview (FV10-ASW 4.1) software. DRG sections were scanned using camera settings identical for all genotypes in a given experiment. Images were imported into the Fiji version (www(dot)//fiji(dot)sc) of ImageJ for threshold subtraction and subsequent analyses as detailed below.

Fluorescence Intensity Analysis: Line scan analysis of fluorescence intensity was carried out on high-resolution confocal z-stack from DRG sections to determine fluorescence intensity of c-FOS and importin α3 over cell regions of interest in-vivo. Briefly, the measurement (in pixels) was effected using the ImageJ drawing tool to draw a line across a neuronal segment covering both cytoplasm and nucleus. All collected traces were then averaged for each experimental group. For comparison of nuclear and cytoplasmic staining intensity 8-bit images of either DRG sections or DRG cultured neurons were processed using the Fiji software. The integrated density was calculated as the sum of the values of the pixels in both cytoplasmic and nuclear regions of interest determined on the basis of TuJ-1, CGRP, TRPV1 or DAPI staining, respectively.

Analyses of transduction efficiency: AAV9 or AAV-PHP.S-driven transduction efficiency was determined using high-resolution confocal z-stack images from DRG and Spinal cord sections from animals injected with the appropriate AAV vector expressing GFP and either shCtrl or shα3. Images were converted to 8-bit, thresholds were defined, and the number of GFP/TuJ-1 double positive neurons counted using the ImageJ cell counter plugin. Cell numbers were expressed as percentage of GFP-positive neurons in the lumbar ventral horn and L4 DRGs.

Neuronal cultures—Adult mouse DRG neurons were cultured as previously described [Perry et al. Neuron 75, 294 (2012)], with plating on poly-L-lysine and laminin coated plates or glass cover slips for 24 hours. Where required, L3-L5 DRG neurons from the uninjured side served as controls for cultures from SNI mice.

Proximity Ligation Assay (PLA) in DRG cultures—The Proximity Ligation Assay (PLA) is used to detect spatial proximity within ˜40 nm of two proteins of interest [O. Söderberg et al., Nature Methods 3, 995-1000 (2006)]. DRG neurons were cultured for 24 hours and fixed for 20 minutes in 4% PFA before blocking and permeabilization with 5% Donkey Serum, 1% BSA, 0. 1% Triton X-100 in PBS for 1 hour. They were then incubated with anti-c-Fos (mouse monocolonal 1:1000, Abcam ab208942) and anti-importin α3 (rabbit polyclonal, 1:2000, Michael Bader lab′, MDC Berlin) overnight at 4° C. PLA was performed using Duolink (Sigma: PLA probe anti-mouse minus DU092004, anti-rabbit plus DU092002 with detection using Far-Red DU092013), according to the manufacturer's instructions. Identification of PLA signal within neurons was effected by subsequent immunostaining with goat anti-CGRP (AbD SEROTEC 1720-9007, IF 1:1000) for 60 minutes at room temperature, followed by three washes and an additional 60 minutes incubation with donkey anti-goat Alexa Fluor 488 (Jackson Immunoresearch, 1:1000). Cells were then washed, mounted with Flouromount-G (ThermoFisher Scientific, cat. #00-4958-02) and imaged by confocal microscopy (Olympus FV1000, 60× oil-immersion objective Olympus UPLSAPO-NA 1.35). PLA signals were quantified by counting puncta in ImageJ.

Western blots from DRG neurons—Cultured DRG neurons were lysed directly in Laemmli buffer and analyzed by Western blot using 5% BSA for blocking and overnight incubations with the following antibodies: anti-importin alpha3 1:5000 (rabbit polyclonal, Bader group, MDC Berlin), anti-c-Fos 1:1000 (mouse monoclonal, clone 2H2, Abcam ab208942), anti-TRPV1 (rabbit polyclonal, Alomone #ACC-030, 1:500). Blots were developed using Radiance ECL (Azure) or SuperSignal™ West Femto (Thermo Scientific) chemiluminescence substrates and quantified with Fiji.

Proximity biotinylation—Proximity biotinylation [K. J. Roux et al. Journal of Cell Biology 196, 801-810 (2012)] was performed by transfecting fusion constructs with the miniTurbo enzyme [T. C. Branon et al., Nat Biotechnol 36, 880-887 (2018)] in N2a cells. Transfections were done with jetPEI™ (Polyplus-transfection), and labelling with 500 μM biotin was initiated 48 hours after transfection. Labeling was stopped after 6 hours by transferring the cells to ice and washing five times with ice-cold PBS. Lysis and streptavidin affinity purification were as previously described [K. J. Roux et al. Journal of Cell Biology 196, 801-810 (2012)].

Transcriptome and Gene Expression Analyses—

Library Construction and Sequencing: Total RNA was extracted from DRGs (embryos and adult tissue) dissected from >3 animals per group using the RNAqueous-Micro Kit (Ambion). Replicates of high RNA integrity (RIN≥6 or 7) were processed for RNA-seq at the Crown Institute for Genomics (G-INCPM, Weizmann Institute of Science). 260 ng or 500 ng of total RNA from each sample was processed using a polyA-based RNA-seq protocol (INCPM mRNA Seq). Sequencing libraries were barcoded to allow multiplexing of 16 or 18 samples on two lanes of Illumina HiSeq, using the Single-Read 60 protocol (v4). The output was ˜31 or ˜27 million reads per sample. Fastq files for each sample were generated with bc12fastq-v2.17.1.14.

Sequence Data Analysis: Poly-A/T stretches and Illumina adapters were trimmed from the reads using cutadapt; and reads shorter than 30 bp were discarded. Reads for each sample were aligned independently using TopHat2 (v2.0.10) against the mouse genome (mm10). The percentage of the reads that were aligned uniquely to the genome was ˜94% (WT vs alpha-3 KO Embryonic dataset) and ˜78% (WT vs alpha-3 KO SNI). Counting proceeded over genes annotated in RefSeq release mm10 and/or Ensembl release 92, using htseq-count (version 0.6.1p1). Only uniquely mapped reads were used to determine the number of reads falling into each gene (intersection-strict mode). Differential analysis was performed using DESeq2 package (1.6.3)[Anders & Huber. Genome Biol 11, R106 (2010)] with the betaPrior, cooksCutoff and independent filtering parameters set to False. Differentially expressed genes were determined by a FDR (p-adjusted)<0.1 (WT vs alpha-3 KO Embryonic dataset), FDR (p-adjusted)<0.05 (WT vs alpha-3 KO SNI) with absolute fold changes >1.5 and max raw counts >10 (WT vs alpha-3 KO Embryonic dataset) and max raw counts >30 (WT vs alpha-3 KO SNI). Raw p values were adjusted for multiple testing using the procedure of Benjamini and Hochberg.

Transcription Factor Binding Site (TFBS) analysis: Possible enrichment of different TFBS in datasets of regulated genes was assessed using FMatch (geneXplain) on gene sets with fold changes of two or more and their corresponding background sets. Promoter sequences from the importin α3 null dataset and a list of background genes (non-deregulated genes) were scanned from 600 base pairs (bp) upstream to 100 bp downstream of the predicted transcription start site for each gene, and TFBS were identified with the TRANSFAC FMatch tool. TFBS enrichment in test versus background sets was assessed by t test with p-value threshold of 0.05.

Gene expression analysis by RT-qPCR: Total RNA from DRG neuronal cultures and DRG tissue from SNI mice were extracted using the Ambion RNAqueous-Micro total RNA isolation kit (Life Technologies Corp.). RNA purity, integrity (RIN>8) and concentration was determined, and 100-200 ng of total RNA was then used to synthesize cDNA using SuperScript III (Invitrogen). RT-qPCR was performed on a ViiA7 System (Applied Biosystems) using PerfeCTa SYBR Green (Quanta Biosciences, Gaithersburg, USA). Forward/Reverse primers were designed for different exons and the RNA was treated with DNase H to avoid false-positives. Amplicon specificity was verified by melting curve analysis. All RT-qPCR reactions were conducted in technical triplicates and the results were averaged for each sample, normalized to Actb levels and the relevant reporter genes such as GFP and for the viruses; and analyzed using the comparative ΔΔCt method [Livak & Schmittgen. Methods 25, 402 (2001)]. The following primers (Mus musculus) were used:

(SQ ID NO: 1)
Actb - F: GGCTGTATTCCCCTCCATCG
AND
(SEQ ID NO: 2)
R: CCAGTTGGTAACAATGCCATGT,
(SEQ ID NO: 3)
Kpna4/importina3 - F: CCAGTGATCGAAATCCACCAA,
and
(SEQ ID NO: 4)
R: CGTTTGTTCAGACGTTCCAGAT,
(SEQ ID NO: 6)
GFP - F: ACGTAAACGGCCACAAGTTC
and
(SEQ ID NO: 7)
R: GTGTACTTCGTCGTGCTGAA;
(SEQ ID NO: 9)
Syngap1 - F: GGGACAAATGGATTGAGAATCTG
and
(SEQ ID NO: 10)
R: GGCGGCTGTTGTCCTTGTT;
(SEQ ID NO: 11)
Slc38a1 - F: ACTTCCTGACGGCCATCTTT
and
(SEQ ID NO: 12)
R: GTCGCCTGTGCTCTGGTACT;
(SEQ ID NO: 13)
Gpr151 - F: GCATGCTTCGCGTATGCA
and
(SEQ ID NO: 14)
R: GATGGTGCGGCTGTGGATA;
(SEQ ID NO: 15)
Rtl1 - F: CCGCTTTCGGTATCACAACA
and
(SEQ ID NO: 16)
R: CGGTCTGGCGATGGAACT.

Viral constructs—generation and validation—AAV shRNA constructs were based on AAV-shRNA-ctrl (Addgene #85741) with specific shRNA sequences cloned in using BamHI and XbaI restriction sites. The target sequence selected for importin α3 was

(SEQ ID NO: 8)
GATCCGGCTTTGACAAACATTGCATGAAGCTTGATGCAATGTTTGT
CAAAGCCTTTTTT.

Sequences of additional targets used were as follows:

shFos 1 -
(SEQ ID NO: 17)
GATCCGGCGGAGACAGATCAACTTGAAGAAGCTTGTTCAAGTTGATCT
GTCTCCGCCTTTTTTT
shFos 2 -
(SEQ ID NO: 18)
GATCCGGGACCTTACCTGTTCGTGAAACGAAGCTTGGTTTCACGAACA
GGTAAGGTCCCTTTTTTT
shJun -
(SEQ ID NO: 19)
GATCCGGCACATCACCACTACACCGACCCCCACCCGAAGCTTGGGGTG
GGGGTCGGTGTAGTGGTGATGTGCCTTTTTTT

For overexpression experiments, an AAV backbone was generated, driving expression from a human Synapsin I (hSynI) promoter to ensure neuronal specificity [M. Mahn et al., Nat Commun 9, 4125 (2018)]. The AAV backbone was modified by inserting a multiple cloning site between hSyn and WPRE, which was then used to introduce the following inserts:

1) A dominant negative A-Fos sequence [M. Olive et al., J Biol Chem 272, 18586-18594 (1997)] obtained from Addgene (plasmid #33353) was amplified with added restriction sites for AscI and EcoRV, and inserted into the AAV backbone, generating pAAV-hSyn-A-Fos-WPRE.
2) Mouse importin α3 open reading frame (ORF) was amplified from mouse brain cDNA using Phusion DNA polymerase and cloned into an AAV backbonespecified above to generate pAAV-hSyn-Importin α3-WPRE.
3) Control constructs contained an EGFP insert, designated pAAV-hSyn-EGFP-WPRE.

Transfection and Western blot analysis: Knockdown and overexpression constructs were tested in HEK or N2A cells transfected using JetPEI (Polyplus) according to manufacturer's instructions and lysed 48 hours later in RIPA buffer supplemented with protease inhibitors (Complete, Roche). For Western blot analysis, 5 μg of protein was separated on TGX Protean 5-15% gradient gels (Biorad) and transferred to nitrocellulose membranes. Membranes were blocked with 5% dried milk-TBST and probed overnight with an importin α3 antibody (1:5000) and GAPDH (MAB374, Millipore, 1:5000) as a loading control in 2% milk-TBST, followed by an anti-mouse HRP-conjugated antibody (#1706516 Biorad). Chemiluminescence was detected with Amersham Imager 600 and band intensities were quantified using the built-in software. AAV production and intrathecal injection: Purified adeno-associated virus (AAV) or the peripheral neuron specific PHP.S (AAV-PHP.S) [K. Y. Chan et al., Nat Neurosci 20, 1172-1179 (2017)] was produced in HEK 293T cells (ATCC®), with the AAVpro® Purification Kit (All Serotypes) from TaKaRa (#6666). For each construct ten 15 cm plates were transfected with 20 μg of DNA (AAV-plasmid containing the construct of interest and two AASV9 or AAV-PHP.S helper plasmids) using jetPEI® (Polyplus) in DMEM medium without serum or antibiotics. The vectors used were pAAV2/9n and pAdDeltaF6 helper vectors (Limberis M P and Wilson J M, Proc Natl Acad Sci USA. 2006 Aug. 29; 103(35):12993-8), pAAV2/9 ((addgene plasmid #112865) pPHP.S helper plasmid (Addgene, plasmid #103006). Medium (DMEM, 20% FBS, 1 mM sodium pyruvate, 100 U/mL penicillin 100 mg/mL streptomycin) was added on the following day to a final concentration of 10% FBS and extraction was effected at three days post transfection. Purification was performed according to the manufacturer's instructions. For all constructs, titers in the range of 10¹²-10¹³ viral genomes/ml were obtained, which were used undiluted for intrathecal injections into the lumbar segments of the spinal cord (5 μl/animal).

Statistical Analyses—All data underwent normality testing using the Shapiro-Wilk test. Potential outliers were discarded using the ROUT method with a Q (maximum desired false discovery rate) of 1%. Datasets that passed the normality test were subjected to parametric analysis. Unpaired Student's t-test was used for analyses with two groups, one-way ANOVA was used to compare multiple groups and two-way ANOVA was used to compare mice over time. In the follow-up analyses, all experimental conditions were compared to one control condition using Tukey's or Dunnett's multiple comparisons tests. Datasets that did not pass the normality test were subjected to nonparametric analysis using the Kruskal-Wallis test on rank for multiple group statistical evaluation followed by Dunn's multiple comparisons test. For 2-groups analyses, the Mann-Whitney test was used. The results are expressed throughout as mean±standard error of the mean (SEM). All analyses were performed using GraphPad Prism version 7.00 for Windows (GraphPad Software, La Jolla, Calif., USA, www(dot)graphpad(dot)com). Statistically significant p values are shown as * p<0.05, ** p<0.01, *** p<0.001 and **** p<0.0001.

### Example 1

### Downregulation of Importin α3 Reduces Pain Sensitivity to Noxious Heat, Chemically-Induced and Neuropathic Pain

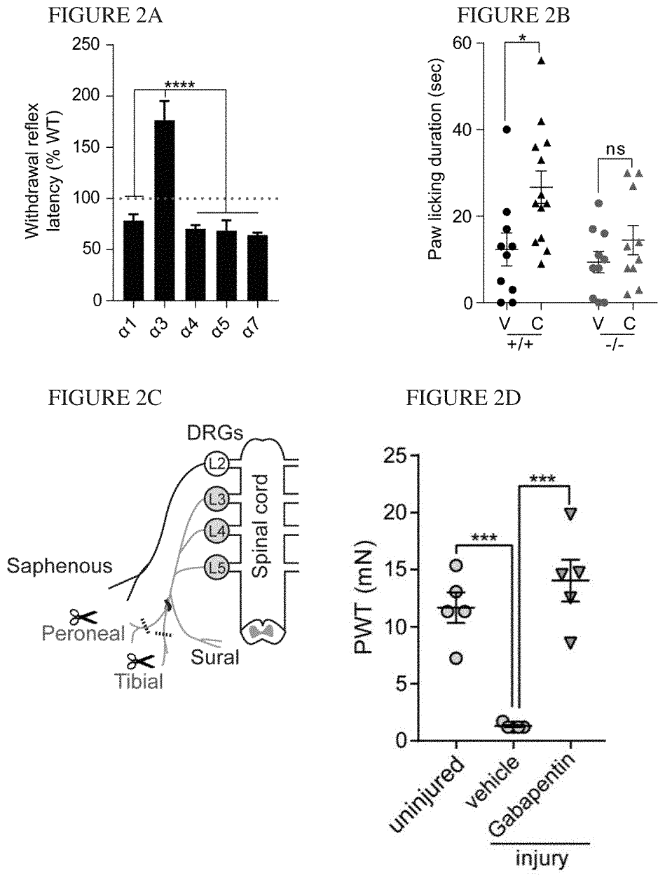

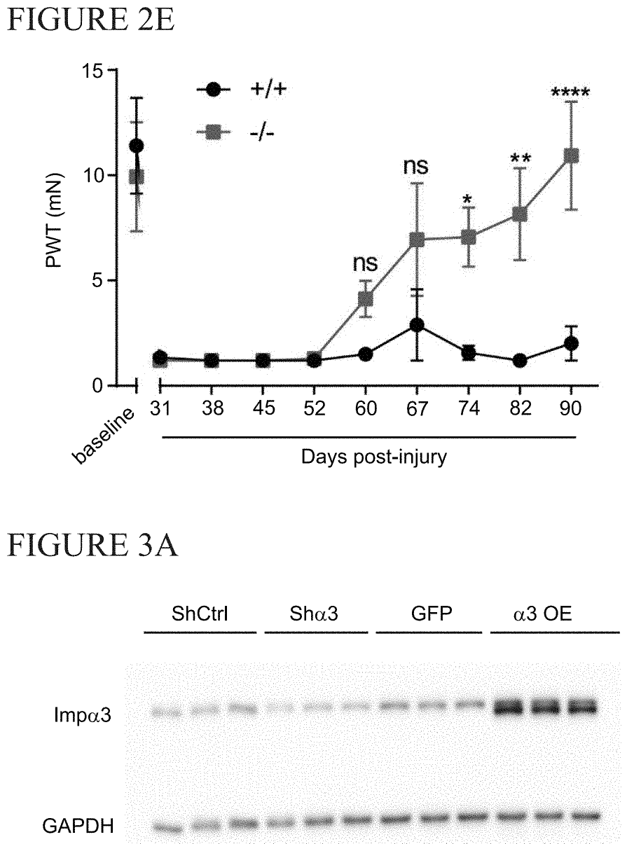

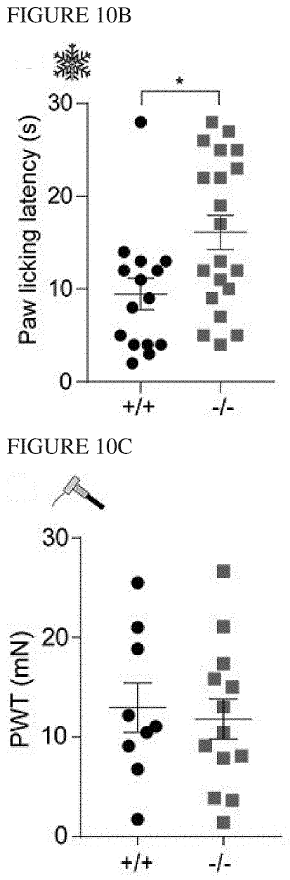

Motor and sensory functions in five different importin α knockout mouse lines^{7,10-12} were examined. Multiple lines revealed mixed sensorimotor phenotypes (FIGS. 1A-C), while only importin α3 null animals exhibited an attenuated response to noxious heat (FIGS. 2A and 10A) and mild attenuation of cold sensitivity (FIG. 10B), without any appreciable effect on basal mechanosensation (FIG. 10C). Acute pain responses were further investigated by capsaicin injection in foot pads, and reduced responses to this chemical induction of pain were observed in importin α3 null animals as compared to age-matched wild-type animals (FIGS. 2B and 1D), although capsaicin did not alter basal mechanosensitivity in these mice (FIG. 10D). In a third assay, the impact of importin α3 knockout on neuropathic pain was evaluated using the spared nerve injury (SNI) model^{13,14} (FIG. 2C). Following, SNI was established in wild-type and importin α3 animals, with periodic monitoring of mechanosensitivity for a total period of three months. As shown in FIG. 2E, responses were similar in wild-type and importin α3 nulls over the first 52 days. However, from day 60 onwards the importin α3 null animals exhibited increasing tolerance to SNI, with less hypersensitivity to touch (FIG. 2E) and reduced unevoked paw clenching (FIG. 11), while wild-type animals did not show any improvement over the entire assay period.

Thus, these data reveal reduced pain sensitivity in importin α3 null mice in three different paradigms including noxious heat, chemically-induced and neuropathic pain.

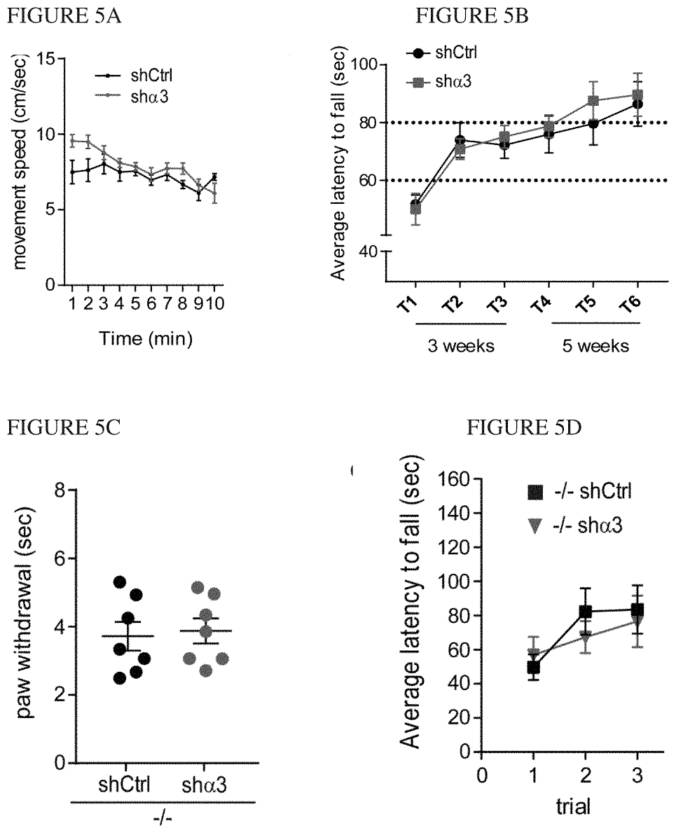

To corroborate these findings in an acute knockdown model in adult animals, intrathecal virus-mediated delivery of shRNAs was used. AAV9 vectors expressing control or anti-importin α3 shRNAs were first tested for knockdown efficacy in culture (FIGS. 3A-B). Following, responsiveness to noxious heat was examined in mice that received validated shRNA constructs. Importin α3 knockdown mice indeed revealed delayed paw withdrawal latency to noxious heat in comparison to mice that received control shRNA (FIG. 4A). Importin α3 knockdown had no effect on exploratory behaviour or motor coordination (FIGS. 5A-B). In addition, importin α3 knockdown using shRNA constructs had no further effect on responses to noxious heat in importin α3 knockout mice (FIGS. 6F-G), confirming the specificity of the findings.

In the next step, the present inventors tested if acute expression of importin α3 could change the reduced pain responsiveness in importin α3 knockout mice. Indeed, increasing importin α3 levels in sensory ganglia in the importin α3 knockout mice increased sensitivity to noxious heat, but did not affect wild type mice (FIG. 4B).

Taken together, these experiments confirm that loss of importin α3 in sensory neurons has specific effects on responses to noxious stimuli.

The effect of acute importin α3 knockdown was further evaluated in the SNI model of neuropathic pain (FIG. 4C), with monitoring using both Von Frey tests for mechanosensitivity and the Catwalk gait analysis system to assess usage of the injured limb. At 60 days post-injury, control shRNA treated SNI mice typically displayed a spontaneous clenched paw phenotype associated with reduced paw print width in the Catwalk assay, while animals treated with anti-importin α3 shRNA regained unaltered paw morphology and gait parameters (FIG. 4D). Mechanosensitivity assays showed that the neuropathic pain response developed in a similar manner in control and anti-importin α3 shRNA treated mice up to 60 days following injury. However, from day 60 onward, importin α3 knockdown animals exhibited a significant recovery of the paw withdrawal reflex, in contrast to no significant change in the control animals (FIG. 4E).

Thus, acute knockdown of importin α3 in adult sensory ganglia also phenocopies the reduced sensitivity to neuropathic pain observed in importin α3 knockouts.

### Example 2

### Mechanism of the Effects of Importin α3 Knockout on Pain Sensitivity

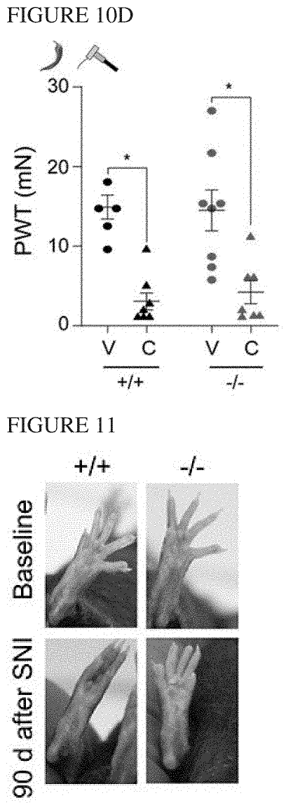

Since different cell types can participate in neuropathic pain circuits [H. Abdo et al., Science 365, 695-699 (2019); X. Yu et al., Nature Communications 11, 264 (2020)], the present inventors checked if the effects of importin α3 on neuropathic pain arise specifically in sensory neurons. To this end, viral transduction of shRNA using AAV-PHP.S, a capsid subtype developed for peripheral neuron specificity [K. Y. Chan et al., Nat Neurosci 20, 1172-1179 (2017)], were carried out. Specificity of AAV-PHP.S was verified by lumbar intrathecal injection, observing efficient transduction of DRG sensory neurons and no expression of the transduced reporter in non-neuronal cells or in central neurons within the spinal cord (FIGS. 12A-H). Following, the effects of importin α3 knockdown by AAV-PHP.S delivery of shRNA after SNI induction was tested (FIG. 13A), monitoring both evoked (FIG. 13B) and unevoked (FIGS. 13C-D) responses to neuropathic pain. Indeed, both the evoked and spontaneous parameters reveal that sensory neuron-specific knockdown of importin α3 provides relief from neuropathic pain, also when knockdown is initiated only after establishment of the pain model.

Mechanistic insight on the effects of importin α3 knockout was then sought by transcriptome analyses in dorsal root ganglia (DRG), with initial analyses on E13.5 embryonic DRG to focus on early stage nociceptors and further analyses on the role of importin α3 in chronic pain using the Spared Nerve Injury model (SNI) in adult mice. The embryo DRG datasets identify genes with changed expression in the absence of importin α3, while the adult SNI model highlights genes with changed expression at the chronic pain stage (2.5 months post-injury) versus early injury stage (7 days post-injury).

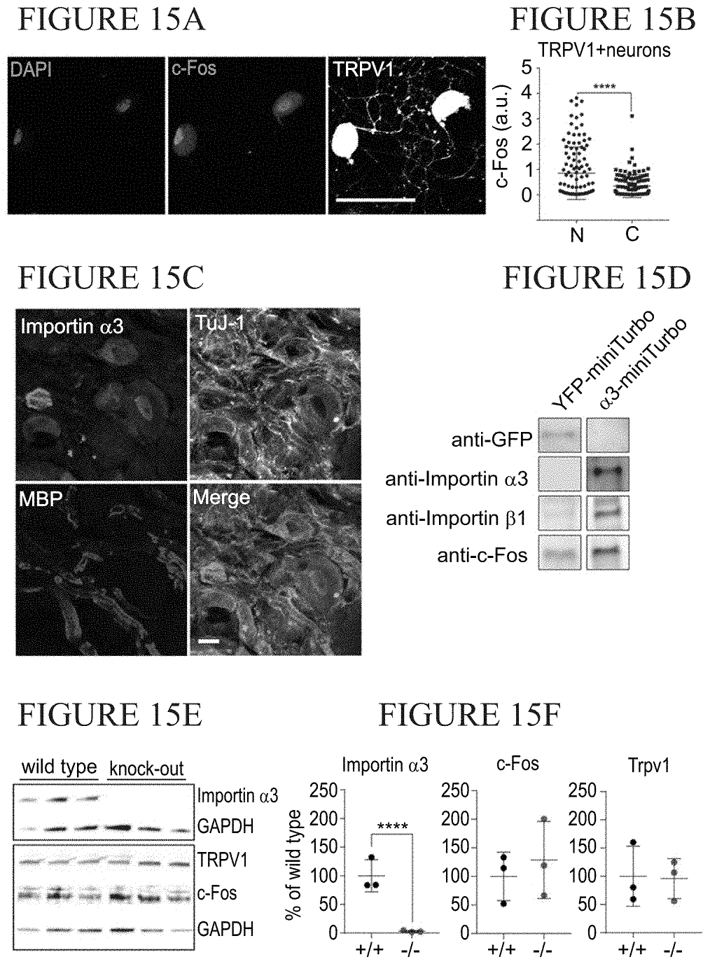

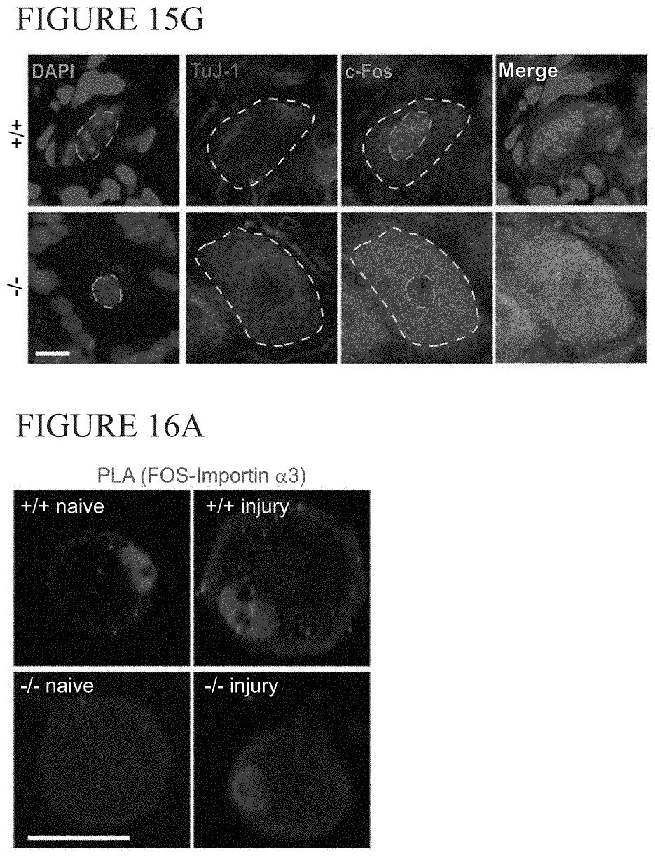

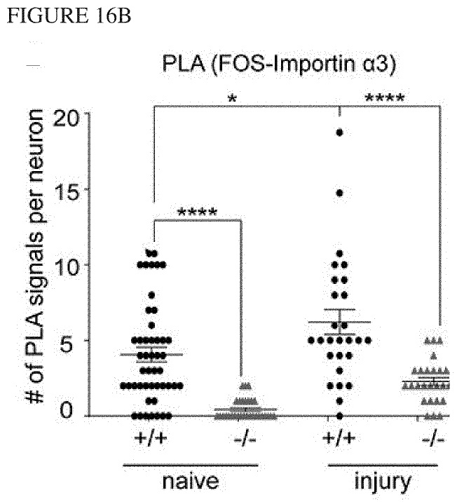

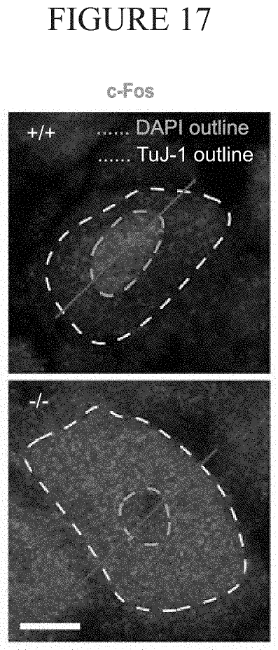

Investigation of differentially expressed gene-sets from RNA-seq (FIGS. 6A and 6C and Tables 4-5 hereinbelow) using the FMatch promoter analysis tool (TRANSFAC, geneXplain) revealed signatures for a number of transcription factors affected by depletion of importin α3 (FIG. 6B). Among these, the AP1 family was prioritized for further studies, since the transcription factor c-Fos is a well-documented marker for pain circuits^{16,17}, reported to regulate expression of the pronociceptive peptide dynorphin¹⁸. Indeed, quantitative analysis of expression regulation of four AP1 target genes following SNI revealed reduced expression of Syngap1 and RTL1 in importin α3 null DRG in comparison with wild type (FIG. 14). In this context it is interesting to note that Syngap1 was previously implicated in tactile sensory processing [S. D. Michaelson et al., Nature Neuroscience 21, 1-13 (2018)]. The transcription factor c-Fos features both a canonical importin α binding nuclear localization signal (NLS) and a binding domain for transportin, an importin β family member with independent nuclear import capability¹⁹. Multiple members of both these nuclear import factor families are widely expressed in sensory neurons [N. Sharma et al., Nature 577, 392-398 (2020)]. Importin α3 and c-Fos expression in DRG neurons was confirmed (FIGS. 15A-C), and their interaction was verified by proximity biotinylation in transfected N2a cells (FIG. 15D) and proximity ligation assay (PLA) of endogenous proteins in sensory neurons (FIGS. 16A-B). Importantly, basal c-Fos expression levels are not changed in importin α3 knockout neurons (FIGS. 15E-F).

Consequently, the subcellular localization of c-Fos in the cell bodies of wild type and importin α3 null sensory neurons was quantified using immunohistochemistry. c-Fos immunostaining appeared mostly nuclear in neuronal somata from wild type adult DRG sections, while in contrast no clear c-Fos nuclear accumulation could be observed in neuronal somata in importin α3 null sections (FIGS. 6F-G, 15G and 17). Similar results were obtained in adult DRG neuronal cultures plated for 24 hours (FIGS. 6H-I). Overall, these analyses showed that importin α3 is required for c-Fos nuclear accumulation in adult sensory neurons.

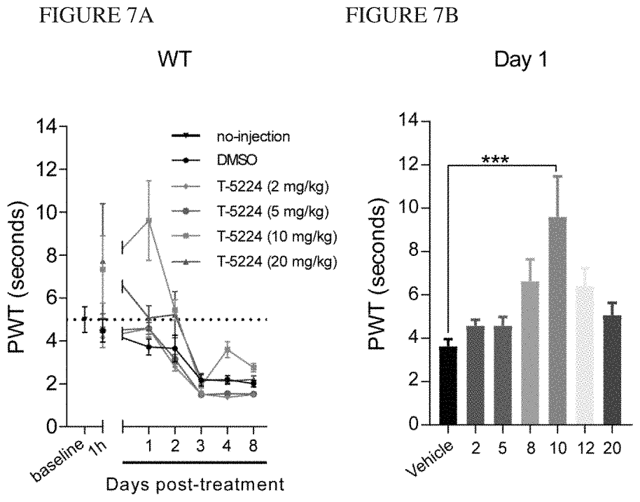

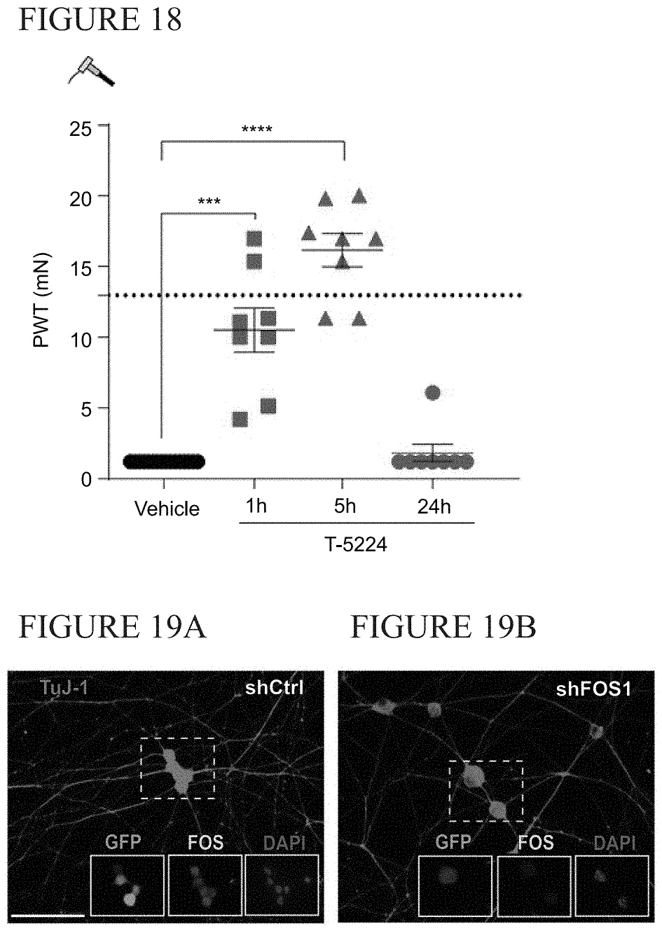

A c-Fos inhibitor termed T-5224 was identified by Aikawa and colleagues²⁰, and has been evaluated for potential analgesic efficacy in intervertebral disc degeneration associated pain²¹. To this end, the effects of T-5224 on responses to noxious heat were compared in wild type versus importin α3 null mice. T-5224 treatment reduced paw withdrawal in response to noxious heat in wild-type mice (FIGS. 7A-B), while it had no additional effect beyond the already existing attenuation in importin α3 null animals (FIGS. 6J-K). Of note, T-5224 did ameliorate the paw withdrawal latency in Von Frey tests of wild type animals one week following induction of SNI (FIG. 18), a time point where importin α3 knockout or knockdown still has no effect on SNI responses (FIGS. 2E, 4E and 13B). These findings provided further support that the analgesic effects of importin α3 depletion are likely due to perturbation of the nuclear import of c-Fos, and suggest that the role of importin α3 is critical mainly in the later maintenance stage of neuropathic pain.

To further corroborate that the AP-1 pathway is required for development of late stage neuropathic pain in the SNI model, the effects of shRNA-mediated knockdown of c-Fos or c-Jun was tested (FIGS. 19A-G). Similarly to the effects of importin α3 depletion, intrathecal delivery of AAV9 expressing c-Fos shRNAs reduced sensitivity to noxious heat (FIG. 20A) without any effect on basal mechanosensitivity (FIG. 20B). c-Jun knockdown reduced sensitivity to both noxious heat and mechanical stimuli (FIG. 20A-B). Following, the effects of c-Fos, c-Jun and importin α3 knockdowns were compared in the SNI model, by intrathecal injection of AAVs expressing the appropriate shRNAs 40 days after initiation of SNI (FIG. 20C). All three knockdowns significantly attenuated the neuropathic pain response 60-90 days post-injury (FIG. 20C).0 In order to test this finding by an independent approach, the effects of a dominant-negative form of AP-1 termed A-Fos [M. Olive et al., J Biol Chem 272, 18586-18594 (1997)], expressed by intrathecal injection of AAV9 under control of the neuron-specific SynapsinI promoter, was examined. Specific overexpression of A-Fos in sensory neurons significantly attenuated noxious heat sensitivity without affecting basal mechanosensitivity (FIGS. 20D-E). Similarly to the knockdown experiments, overexpression of A-Fos 40 days following SNI significantly reduced the neuropathic pain response in the 67-90 days assay window (FIG. 20F). Taken together, these findings confirm that AP-1 pathway inhibition attenuates neuropathic pain in the SNI model.

TABLE 4
Genes differentially expressed in importin α3 null (−/−) as
compared to WT (+/+) mice, as determined by RNA-seq using the
FMatch promoter analysis tool (TRANSFAC, geneXplain).

| | | | log2Fold | Fold | | | |
|---|---|---|---|---|---|---|---|
| Gene symbol | max | Base Mean | Change | Change | LinearFC | pvalue | padj |
| Kpna4 | 2157 | 1338.695 | −6.4622778 | 0.0113412 | −88.173781 | 0 | 0 |
| Gsx1 | 129 | 14.159552 | −5.7503948 | 0.0185763 | −53.832101 | 0.000163 | 0.0403557 |
| Gm3893 | 1255 | 310.20005 | −5.1425498 | 0.0283099 | −35.323339 | 5.88E−07 | 0.0022518 |
| Robo3 | 2175 | 319.14008 | −4.6809533 | 0.0389846 | −25.651181 | 9.56E−07 | 0.0022518 |
| Rhbg | 55 | 6.7113735 | −4.455736 | 0.0455711 | −21.943717 | 0.0009567 | 0.0943542 |
| Hoxc11 | 308 | 72.10939 | −4.4213206 | 0.0466713 | −21.426445 | 0.0001102 | 0.0354354 |
| Fgf15 | 256 | 39.611695 | −4.2555528 | 0.0523541 | −19.100689 | 4.08E−05 | 0.0232131 |
| Hotair | 128 | 31.521173 | −4.1698144 | 0.0555598 | −17.998621 | 0.0009534 | 0.0943542 |
| Hc | 602 | 51.59881 | −3.8503709 | 0.0693303 | −14.423715 | 0.0008454 | 0.0902589 |
| Slc6a5 | 120 | 20.365849 | −3.758416 | 0.0738931 | −13.533059 | 0.0001153 | 0.0363655 |
| Gulo | 453 | 41.78467 | −3.7562647 | 0.0740034 | −13.512893 | 0.000343 | 0.0590222 |
| Hoxd11 | 908 | 194.78112 | −3.6304256 | 0.0807482 | −12.384173 | 0.0002814 | 0.0534625 |
| Slc32a1 | 224 | 47.841199 | −3.5896466 | 0.0830632 | −12.039024 | 2.21E−07 | 0.0022518 |
| BC024386 | 32 | 5.7374916 | −3.5726268 | 0.0840489 | −11.897832 | 0.0009072 | 0.0927035 |
| Slc30a3 | 120 | 29.335484 | −3.5282846 | 0.0866723 | −11.537706 | 2.33E−06 | 0.0039819 |
| C130021I20Rik | 561 | 135.48943 | −3.4432661 | 0.0919335 | −10.877432 | 1.00E−06 | 0.0022518 |
| Slc27a2 | 395 | 39.438502 | −3.4233561 | 0.093211 | −10.728349 | 0.000295 | 0.054482 |
| Lbx1 | 152 | 32.793409 | −3.4096005 | 0.094104 | −10.626544 | 0.0001303 | 0.0386973 |
| Skor1 | 578 | 101.09987 | −3.4060716 | 0.0943344 | −10.600582 | 2.15E−05 | 0.0163416 |
| Gm17750 | 59 | 14.986913 | −3.3915427 | 0.0952892 | −10.494363 | 1.81E−05 | 0.015604 |
| Pax2 | 705 | 147.53674 | −3.3685371 | 0.0968209 | −10.328344 | 1.06E−06 | 0.0022518 |
| Nxph1 | 104 | 26.720284 | −3.3450072 | 0.098413 | −10.161258 | 8.02E−05 | 0.032882 |
| Gad1 | 200 | 46.364257 | −3.308904 | 0.1009068 | −9.9101304 | 1.48E−05 | 0.013777 |
| Lhx1 | 375 | 76.963115 | −3.2999136 | 0.1015376 | −9.8485657 | 8.83E−06 | 0.0120644 |
| Stra6l | 100 | 20.587278 | −3.2924198 | 0.1020664 | −9.7975415 | 1.43E−05 | 0.013777 |
| Sall3 | 905 | 155.33186 | −3.125054 | 0.1146212 | −8.7243887 | 0.0005014 | 0.0708799 |
| Lhx5 | 436 | 92.050056 | −3.0814938 | 0.1181348 | −8.4649044 | 3.11E−05 | 0.0212613 |
| Aqp6 | 58 | 14.40217 | −3.0593645 | 0.1199608 | −8.3360535 | 0.0001864 | 0.0429261 |
| Dpep1 | 26 | 6.2574072 | −2.9461875 | 0.1297505 | −7.7070967 | 0.0005418 | 0.0726536 |
| A530058N18Rik | 31 | 5.9112809 | −2.8724457 | 0.136555 | −7.3230553 | 0.001025 | 0.0972719 |
| Sst | 356 | 73.841755 | −2.839028 | 0.139755 | −7.1553782 | 2.09E−05 | 0.0163416 |
| Lmx1b | 128 | 28.739421 | −2.8134736 | 0.1422525 | −7.0297512 | 0.0006505 | 0.0813093 |
| Cacng3 | 252 | 40.793428 | −2.8044069 | 0.1431494 | −6.9857107 | 0.0007726 | 0.0846256 |
| Foxb1 | 83 | 19.751301 | −2.8039065 | 0.143199 | −6.9832883 | 0.0001412 | 0.0398753 |
| Lamp5 | 655 | 133.41583 | −2.7885079 | 0.1447356 | −6.9091486 | 0.0001039 | 0.0345891 |
| Pax8 | 292 | 58.241885 | −2.6853699 | 0.1554616 | −6.432457 | 9.96E−07 | 0.0022518 |
| Krt8 | 390 | 54.64426 | −2.6528323 | 0.1590076 | −6.2890073 | 0.000676 | 0.0815126 |
| Mab21l1 | 361 | 92.469517 | −2.6456014 | 0.1598066 | −6.2575653 | 7.14E−05 | 0.032882 |
| Myh8 | 1391 | 251.97644 | −2.6388749 | 0.1605534 | −6.2284576 | 0.0010597 | 0.0991879 |
| Lmo3 | 106 | 26.383087 | −2.6161588 | 0.1631014 | −6.1311547 | 0.0002658 | 0.0524928 |
| Slc18a3 | 255 | 46.553755 | −2.6090022 | 0.1639125 | −6.1008158 | 0.0007337 | 0.0846256 |
| Krt14 | 160 | 37.846468 | −2.6082631 | 0.1639965 | −6.0976914 | 1.12E−05 | 0.0131988 |
| Dbx1 | 55 | 13.562081 | −2.5828275 | 0.1669135 | −5.9911274 | 0.000964 | 0.0943542 |
| Cldn4 | 36 | 11.270589 | −2.5555268 | 0.1701021 | −5.878821 | 5.46E−05 | 0.0287083 |
| Irx6 | 46 | 12.100708 | −2.5514106 | 0.1705882 | −5.8620717 | 0.0008753 | 0.0910758 |
| Myh7 | 1064 | 173.39215 | −2.5206492 | 0.1742645 | −5.7384027 | 7.58E−05 | 0.032882 |
| Chrnd | 123 | 20.530166 | −2.4944712 | 0.1774555 | −5.6352172 | 0.0002353 | 0.0487234 |
| Xirp1 | 93 | 16.8619 | −2.479395 | 0.1793196 | −5.5766355 | 0.0005853 | 0.0759399 |
| Gad2 | 260 | 59.204253 | −2.4228664 | 0.1864853 | −5.3623538 | 0.0001044 | 0.0345891 |
| Gjd4 | 64 | 12.043851 | −2.4205072 | 0.1867905 | −5.3535918 | 0.0009135 | 0.0927035 |
| Mlc1 | 34 | 13.328819 | −2.3991557 | 0.1895755 | −5.2749437 | 0.0001218 | 0.0372644 |
| 5430431A17Rik | 225 | 56.063242 | −2.3777899 | 0.1924039 | −5.1973993 | 4.85E−05 | 0.0261366 |
| Tacstd2 | 38 | 14.423617 | −2.3777292 | 0.192412 | −5.1971808 | 8.72E−05 | 0.0337278 |
| Wnt7b | 408 | 98.312256 | −2.3146576 | 0.2010104 | −4.9748658 | 0.0003692 | 0.060661 |
| Cartpt | 697 | 143.25869 | −2.3116467 | 0.2014304 | −4.9644941 | 6.06E−05 | 0.0310591 |
| Pdyn | 72 | 26.089907 | −2.2720176 | 0.2070401 | −4.8299812 | 6.23E−05 | 0.0311341 |
| Ttn | 1938 | 424.42542 | −2.2354103 | 0.2123609 | −4.7089659 | 0.0009712 | 0.0943542 |
| Pax7 | 133 | 32.806011 | −2.1984021 | 0.2178788 | −4.5897071 | 0.0003804 | 0.060877 |
| Pou3f2 | 538 | 115.06346 | −2.1824231 | 0.2203054 | −4.539153 | 2.70E−05 | 0.0190929 |
| Gabrp | 91 | 32.397564 | −2.1698383 | 0.2222356 | −4.4997295 | 0.0002551 | 0.051781 |
| Lad1 | 17 | 10.231705 | −2.1600266 | 0.2237521 | −4.469231 | 6.67E−05 | 0.0325781 |
| Krt5 | 271 | 120.29861 | −2.1558177 | 0.2244059 | −4.4562116 | 3.55E−05 | 0.0221238 |
| 1110015O18Rik | 26 | 10.42051 | −2.1557783 | 0.224412 | −4.4560898 | 0.0004234 | 0.0633537 |
| Dsp | 228 | 53.671456 | −2.1500148 | 0.2253103 | −4.4383233 | 4.07E−05 | 0.0232131 |
| Trp63 | 79 | 21.771647 | −2.1499126 | 0.2253263 | −4.4380089 | 1.86E−05 | 0.015604 |
| D030068K23Rik | 35 | 12.059737 | −2.1037373 | 0.2326548 | −4.2982139 | 0.0001556 | 0.0403557 |
| Krt17 | 71 | 24.531263 | −2.0940078 | 0.2342291 | −4.2693244 | 0.0001682 | 0.0403557 |
| Barhl1 | 70 | 27.731073 | −2.0904096 | 0.234814 | −4.2586897 | 0.0001539 | 0.0403557 |
| Mybph | 614 | 118.86424 | −2.089695 | 0.2349304 | −4.2565807 | 0.000676 | 0.0815126 |
| Ecel1 | 321 | 103.41152 | −2.0636843 | 0.2392044 | −4.1805255 | 0.0003532 | 0.0593477 |
| Actn2 | 2165 | 426.88778 | −2.0351196 | 0.2439877 | −4.0985671 | 0.0001046 | 0.0345891 |
| Krt7 | 70 | 28.474025 | −2.0120208 | 0.2479256 | −4.0334679 | 0.0001999 | 0.0454535 |
| Grin2c | 94 | 20.693472 | −2.0073844 | 0.2487237 | −4.0205263 | 0.0007552 | 0.0846256 |
| Casq2 | 534 | 104.61753 | −1.9512511 | 0.2585919 | −3.8670973 | 0.0009506 | 0.0943542 |
| Arx | 60 | 20.497946 | −1.9481761 | 0.2591436 | −3.8588639 | 0.0005288 | 0.0722634 |
| Neb | 890 | 182.00594 | −1.94404 | 0.2598877 | −3.8478164 | 0.0007738 | 0.0846256 |
| Myom2 | 343 | 70.09626 | −1.9415813 | 0.2603309 | −3.8412645 | 0.0002043 | 0.0455143 |
| Esrp1 | 33 | 15.41179 | −1.9231568 | 0.2636769 | −3.7925199 | 0.0001492 | 0.0402461 |
| Kremen2 | 209 | 53.134513 | −1.9182317 | 0.2645786 | −3.7795952 | 0.000235 | 0.0487234 |
| Plk5 | 13 | 8.9602828 | −1.9029412 | 0.2673977 | −3.7397483 | 0.0007337 | 0.0846256 |
| Hspb2 | 116 | 33.74743 | −1.831164 | 0.2810378 | −3.5582404 | 8.55E−05 | 0.033722 |
| Car14 | 89 | 22.330692 | −1.8169595 | 0.2838185 | −3.5233787 | 0.0007751 | 0.0846256 |
| Tnnt2 | 679 | 233.24899 | −1.7991203 | 0.2873497 | −3.4800796 | 8.01E−05 | 0.032882 |
| Chrdl2 | 29 | 13.175163 | −1.741971 | 0.298961 | −3.3449184 | 0.0003025 | 0.0553733 |
| Fut9 | 127 | 38.401867 | −1.7417512 | 0.2990065 | −3.3444087 | 1.26E−05 | 0.01356 |
| Pkp3 | 35 | 14.698861 | −1.7385958 | 0.2996612 | −3.3371019 | 0.0001352 | 0.0390385 |
| Jsrp1 | 58 | 22.630601 | −1.7265981 | 0.3021636 | −3.3094651 | 1.90E−05 | 0.015604 |
| Slc38a5 | 352 | 97.003254 | −1.7254815 | 0.3023976 | −3.3069049 | 0.0010037 | 0.0960584 |
| Shank1 | 1274 | 431.60153 | −1.7181346 | 0.3039415 | −3.2901071 | 1.25E−06 | 0.0023203 |
| 2210039B01Rik | 162 | 49.113464 | −1.7171308 | 0.304153 | −3.2878188 | 0.0010075 | 0.0960584 |
| Sall1 | 735 | 211.47121 | −1.6934062 | 0.3091961 | −3.2341939 | 0.0002398 | 0.0491552 |
| Ldb3 | 362 | 91.880178 | −1.6296398 | 0.3231689 | −3.0943574 | 0.0002164 | 0.0467787 |
| Myh7b | 451 | 105.55606 | −1.6264212 | 0.3238907 | −3.0874615 | 0.0003385 | 0.0588114 |
| Nrap | 97 | 27.430123 | −1.6224435 | 0.3247849 | −3.0789608 | 0.0001428 | 0.0398753 |
| Smyd1 | 516 | 131.71919 | −1.6197678 | 0.3253878 | −3.0732557 | 0.0009359 | 0.0943542 |
| Nexn | 302 | 90.434334 | −1.617847 | 0.3258213 | −3.0691666 | 0.0005423 | 0.0726536 |
| Krt15 | 68 | 32.061582 | −1.6128764 | 0.3269459 | −3.0586104 | 0.0004298 | 0.0638502 |
| Rmst | 60 | 22.410994 | −1.5961927 | 0.3307487 | −3.0234437 | 0.0005076 | 0.0712635 |
| Gpr179 | 46 | 22.910704 | −1.5693867 | 0.3369516 | −2.9677853 | 9.89E−05 | 0.0345891 |
| Bhlhe22 | 125 | 47.121863 | −1.5588823 | 0.3394139 | −2.946255 | 7.49E−05 | 0.032882 |
| Alk | 161 | 55.682955 | −1.5463289 | 0.3423802 | −2.9207298 | 0.000292 | 0.0544077 |
| Tnnt1 | 1434 | 447.19097 | −1.5417447 | 0.3434698 | −2.9114639 | 0.0004809 | 0.0690524 |
| Dtx1 | 491 | 156.51556 | −1.5366016 | 0.3446965 | −2.9011032 | 8.01E−05 | 0.032882 |
| Mapk13 | 41 | 16.977328 | −1.5149021 | 0.3499202 | −2.8577943 | 0.0007515 | 0.0846256 |
| Inhbb | 60 | 23.185471 | −1.5113481 | 0.3507833 | −2.8507629 | 0.0001106 | 0.0354354 |
| Slco1c1 | 39 | 16.850328 | −1.5041303 | 0.3525426 | −2.8365363 | 0.0001713 | 0.0403557 |
| Gjd2 | 208 | 61.869477 | −1.494454 | 0.3549151 | −2.8175749 | 0.0004164 | 0.0627625 |
| Obscn | 335 | 87.683449 | −1.4451231 | 0.3672608 | −2.7228605 | 0.000594 | 0.0765844 |
| Myom1 | 611 | 152.57784 | −1.4068582 | 0.3771321 | −2.6515909 | 0.0004817 | 0.0690524 |
| Clec7a | 52 | 24.399449 | −1.3661859 | 0.3879154 | −2.5778815 | 0.0001487 | 0.0402461 |
| Ntn1 | 1268 | 311.96021 | −1.3176402 | 0.4011906 | −2.4925807 | 0.0004086 | 0.0627625 |
| Perp | 162 | 66.679039 | −1.3085828 | 0.4037173 | −2.4769811 | 1.16E−05 | 0.0131988 |
| Pak6 | 198 | 100.47759 | −1.2919394 | 0.4084017 | −2.4485699 | 0.0001664 | 0.0403557 |
| Gdf10 | 923 | 348.08299 | −1.2445194 | 0.4220485 | −2.3693962 | 0.000611 | 0.0777976 |
| Tspan11 | 254 | 104.59247 | −1.1844375 | 0.4399961 | −2.2727476 | 0.0002663 | 0.0524928 |
| Slc12a5 | 417 | 171.83163 | −1.1583462 | 0.4480258 | −2.2320142 | 0.0007644 | 0.0846256 |
| Elfn2 | 104 | 54.437502 | −1.1257528 | 0.4582628 | −2.1821539 | 0.0003729 | 0.060661 |
| Chl1 | 834 | 333.9247 | −1.1209325 | 0.4597965 | −2.1748751 | 0.0001251 | 0.0377231 |
| Tmem132d | 81 | 46.111605 | −1.1064216 | 0.4644446 | −2.1531093 | 0.0007019 | 0.0836566 |
| Ankrd35 | 46 | 29.123842 | −1.1026285 | 0.4656673 | −2.1474559 | 0.0003071 | 0.0557064 |
| Gpm6a | 1193 | 515.76714 | −1.0765632 | 0.474157 | −2.1090061 | 0.0001439 | 0.0398753 |
| Fap | 222 | 97.298817 | −1.0621382 | 0.4789217 | −2.0880239 | 0.0007487 | 0.0846256 |
| Fibcd1 | 53 | 30.825355 | −1.0616715 | 0.4790767 | −2.0873485 | 0.0001647 | 0.0403557 |
| Fndc5 | 991 | 524.44347 | −1.058794 | 0.4800332 | −2.0831893 | 0.0004806 | 0.0690524 |
| Crabp1 | 4216 | 1772.594 | −1.031988 | 0.4890358 | −2.0448401 | 0.0005407 | 0.0726536 |
| Sult5a1 | 55 | 33.560444 | −1.0175359 | 0.4939593 | −2.0244583 | 0.0007378 | 0.0846256 |
| Gramd1c | 80 | 48.601539 | −0.99737 | 0.5009123 | −1.9963574 | 0.0001697 | 0.0403557 |
| Tnfrsf11a | 52 | 31.592486 | −0.9784834 | 0.507513 | −1.9703929 | 0.0002263 | 0.047816 |
| Dlg2 | 338 | 172.30921 | −0.9585896 | 0.5145597 | −1.9434091 | 0.0010665 | 0.0993703 |
| Lrrc4b | 1509 | 708.25728 | −0.9476423 | 0.5184791 | −1.9287181 | 4.87E−06 | 0.007129 |
| Kcnj2 | 100 | 53.852086 | −0.9460044 | 0.5190681 | −1.9265296 | 0.0003226 | 0.0580051 |
| Adamts19 | 122 | 65.887877 | −0.9392914 | 0.521489 | −1.9175861 | 0.0005168 | 0.071967 |
| Reln | 1576 | 870.52234 | −0.9154714 | 0.5301706 | −1.8861853 | 0.0002168 | 0.0467787 |
| Anxa1 | 108 | 73.806111 | −0.8689037 | 0.5475628 | −1.8262746 | 0.0007676 | 0.0846256 |
| Slc10a4 | 436 | 197.96236 | −0.8614286 | 0.5504073 | −1.8168365 | 0.0004506 | 0.0664549 |
| Slc16a14 | 65 | 40.968255 | −0.8537074 | 0.5533609 | −1.8071389 | 0.0003934 | 0.0610893 |
| Adgrb2 | 1208 | 778.73169 | −0.8514134 | 0.5542415 | −1.8042677 | 0.0004145 | 0.0627625 |
| Kif26a | 1270 | 675.22829 | −0.8188332 | 0.5669002 | −1.7639788 | 4.83E−05 | 0.0261366 |
| Bhlhe40 | 118 | 68.728822 | −0.8162691 | 0.5679087 | −1.7608465 | 7.70E−05 | 0.032882 |
| Dnah2 | 95 | 55.752629 | −0.8123655 | 0.5694474 | −1.7560884 | 0.0001001 | 0.0345891 |
| AW549542 | 148 | 85.709696 | −0.8090447 | 0.5707597 | −1.7520509 | 0.0001009 | 0.0345891 |
| Fstl5 | 503 | 373.07997 | −0.8011488 | 0.573892 | −1.7424881 | 4.98E−07 | 0.0022518 |
| Fndc1 | 102 | 66.610753 | −0.7976607 | 0.5752812 | −1.7382802 | 0.000766 | 0.0846256 |
| Sorl1 | 422 | 221.24088 | −0.7852833 | 0.580238 | −1.7234308 | 0.0009703 | 0.0943542 |
| Usp29 | 177 | 90.806151 | −0.771456 | 0.5858259 | −1.7069917 | 0.0003831 | 0.060877 |
| Magi2 | 585 | 305.10956 | −0.7611536 | 0.5900244 | −1.6948453 | 3.56E−05 | 0.0221238 |
| Shroom1 | 74 | 49.42511 | −0.7580036 | 0.591314 | −1.6911488 | 0.0001666 | 0.0403557 |
| Camk2n1 | 795 | 463.86859 | −0.7464477 | 0.5960694 | −1.6776569 | 0.0007765 | 0.0846256 |
| Acbd4 | 250 | 131.28602 | −0.7458362 | 0.5963221 | −1.6769459 | 0.0005981 | 0.0766292 |
| Aldh1l1 | 316 | 149.4399 | −0.733322 | 0.6015173 | −1.6624627 | 0.0009028 | 0.0927035 |
| Fgf9 | 109 | 63.188408 | −0.6942245 | 0.6180415 | −1.6180144 | 0.000753 | 0.0846256 |
| Fcrls | 236 | 157.52295 | −0.6622193 | 0.6319055 | −1.5825152 | 3.75E−05 | 0.0226321 |
| Rftn1 | 211 | 148.58955 | −0.6441753 | 0.6398585 | −1.5628456 | 0.0002838 | 0.0534625 |
| Fgfr3 | 561 | 271.8598 | −0.6064924 | 0.6567916 | −1.5225529 | 0.0007802 | 0.0846256 |
| Fblim1 | 2224 | 1813.8662 | −0.6024512 | 0.658634 | −1.518294 | 1.10E−05 | 0.0131988 |
| H19 | 72857 | 54388.304 | −0.6008041 | 0.6593863 | −1.5165617 | 0.0001325 | 0.038813 |
| Six1 | 5837 | 3235.029 | 0.6101587 | 1.5264272 | 1.5264272 | 0.0002805 | 0.0534625 |
| Tbc1d9 | 14341 | 7858.1173 | 0.6102513 | 1.5265251 | 1.5265251 | 0.0009119 | 0.0927035 |
| Sh2d4b | 81 | 46.148682 | 0.72982 | 1.6584321 | 1.6584321 | 0.0003525 | 0.0593477 |
| Kcnmb2 | 3515 | 1816.4384 | 0.7460874 | 1.6772379 | 1.6772379 | 0.0003337 | 0.0588114 |
| Gm20752 | 26 | 10.753811 | 1.661653 | 3.1637882 | 3.1637882 | 0.0003726 | 0.060661 |
| Gm4349 | 36 | 12.567449 | 2.1403416 | 4.4086641 | 4.4086641 | 3.27E−05 | 0.0215917 |
| Atp6v1c2 | 13 | 3.7090941 | 2.1566263 | 4.4587098 | 4.4587098 | 0.0005196 | 0.071967 |
| Cd1d2 | 27 | 7.2289085 | 2.4394314 | 5.4242792 | 5.4242792 | 0.0006578 | 0.0815126 |

TABLE 5
Genes differentially expressed in DRGs of importin α3 null (−/−)
mice 7 days following SNI versus 2.5 months following injury

| | | log2 | | | | |
|---|---|---|---|---|---|---|
| Gene_Symbol | max | FC | Fold Change | pvalue | padj | Direction |
| Scpep1 | 2310 | −1.74 | 0.299369676 | 0 | 0 | down |
| Tpd52l1 | 106 | 1.32 | 2.496661098 | 0.000266 | 0.00702 | up |
| Cttnbp2 | 709 | −1.24 | 0.423372656 | 3.65E−08 | 0.00000529 | down |
| Gm38393 | 221 | −1.6 | 0.329876978 | 0.00000957 | 0.00054 | down |
| S100a4 | 285 | 1.87 | 3.655325801 | 0.000000248 | 0.0000241 | up |
| Cfp | 196 | −1.09 | 0.469761375 | 0.00129 | 0.0221 | down |
| Gabrg1 | 1230 | −1.48 | 0.358488812 | 3.72E−08 | 0.00000535 | down |
| Tubb6 | 2100 | −1.79 | 0.289172046 | 9E−10 | 0.00000021 | down |
| Gstt1 | 400 | 1.56 | 2.948538435 | 0 | 4E−10 | up |
| Gstt3 | 92 | 1.4 | 2.639015822 | 0.000134 | 0.0041 | up |
| Zim1 | 98 | −1.1 | 0.466516496 | 0.00161 | 0.0255 | down |
| Rgs20 | 197 | −1.84 | 0.279321785 | 7.44E−08 | 0.0000096 | down |
| Il17ra | 301 | −1.93 | 0.262429171 | 0.000000782 | 0.0000651 | down |
| Fosb | 129 | 2.1 | 4.28709385 | 0.000157 | 0.00466 | up |
| Man1a | 403 | −1.13 | 0.456915725 | 0.000136 | 0.00413 | down |
| Itpkc | 237 | −1.58 | 0.334481889 | 0.000000072 | 0.0000094 | down |
| Nqo1 | 250 | 1.08 | 2.114036081 | 0.00000287 | 0.000192 | up |
| Hlf | 263 | 1.57 | 2.969047141 | 0.0000631 | 0.00234 | up |
| Stat5a | 275 | −1.25 | 0.420448208 | 0.000000167 | 0.0000177 | down |
| Col5a3 | 8820 | −1.98 | 0.25348987 | 0 | 0 | down |
| Pax2 | 98 | 1.16 | 2.234574276 | 0.00336 | 0.0415 | up |
| Pde1c | 3030 | −1.29 | 0.408951029 | 6E−10 | 0.000000142 | down |
| Sst | 464 | 1.05 | 2.070529848 | 0.00213 | 0.0307 | up |
| Nes | 1190 | 1.41 | 2.657371628 | 0.0000145 | 0.000743 | up |
| Wisp1 | 244 | −1.33 | 0.397768242 | 0.000000962 | 0.0000761 | down |
| Mthfd2 | 777 | −1.24 | 0.423372656 | 0 | 0 | down |
| Tyrp1 | 310 | −1.33 | 0.397768242 | 0.00224 | 0.0318 | down |
| Crip1 | 733 | 1.14 | 2.203810232 | 0.00424 | 0.0483 | up |
| Adamts4 | 36 | −1.52 | 0.348685917 | 0.00109 | 0.0195 | down |
| Id3 | 2600 | 1.03 | 2.042024251 | 0.0000675 | 0.00246 | up |
| Phox2a | 41 | −4.69 | 0.038740866 | 0.000472 | 0.0107 | down |
| G0s2 | 396 | 1.39 | 2.620786808 | 0.00000112 | 0.000088 | up |
| Cox4i2 | 48 | 1.52 | 2.867910496 | 0.00401 | 0.0467 | up |
| Kif19a | 794 | 1.08 | 2.114036081 | 0.00259 | 0.0349 | up |
| Rdm1 | 207 | 1.05 | 2.070529848 | 0.00143 | 0.0235 | up |
| Mcoln2 | 94 | −2.32 | 0.200267469 | 0.0000603 | 0.00226 | down |
| Bcas1 | 1870 | 1.94 | 3.837056477 | 0.000000535 | 0.000047 | up |
| Csf1 | 3040 | −2.46 | 0.181746565 | 0 | 0 | down |
| Abca1 | 6500 | −1.86 | 0.275476279 | 0 | 6E−10 | down |
| Egfl8 | 631 | 1.12 | 2.173469725 | 0.0000998 | 0.0033 | up |
| Fam163a | 148 | −1.52 | 0.348685917 | 2E−10 | 0.000000054 | down |
| Fcrls | 211 | −1.3 | 0.406126198 | 0.00213 | 0.0307 | down |
| Fam184b | 158 | −1.28 | 0.411795509 | 0.00000346 | 0.000227 | down |
| Hsd11b1 | 31 | 2.03 | 4.084048503 | 0.000767 | 0.0152 | up |
| Wt1 | 51 | −2.14 | 0.226879789 | 0.000108 | 0.0035 | down |
| Phf21b | 93 | −1.27 | 0.414659773 | 0.000000663 | 0.0000562 | down |
| Il13ra1 | 476 | −1.27 | 0.414659773 | 2.87E−08 | 0.00000436 | down |
| Gsdma | 37 | −1.54 | 0.343885455 | 0.00411 | 0.0475 | down |
| Jph2 | 115 | 1.04 | 2.056227653 | 0.00181 | 0.0278 | up |
| Kcnab3 | 57 | 1.02 | 2.02791896 | 0.00213 | 0.0307 | up |
| Sema6a | 3440 | −2.26 | 0.20877198 | 0 | 0 | down |
| Crybg1 | 208 | −1.68 | 0.312082637 | 9.76E−08 | 0.0000119 | down |
| Fabp7 | 2930 | −1.14 | 0.453759578 | 0.00000189 | 0.000137 | down |
| Hal | 282 | 1.14 | 2.203810232 | 0.00208 | 0.0304 | up |
| Egfr | 214 | −1.63 | 0.323088208 | 0.0000662 | 0.00242 | down |
| Phlda1 | 1610 | −1.78 | 0.291183397 | 0 | 4E−10 | down |
| Aldh1l2 | 160 | −1.52 | 0.348685917 | 0.000372 | 0.00894 | down |
| Flt4 | 51 | 1.23 | 2.345669898 | 0.00104 | 0.0188 | up |
| Igfbp3 | 1140 | −1.25 | 0.420448208 | 0.000000103 | 0.0000122 | down |
| Smtn | 1650 | 1.56 | 2.948538435 | 0.0000723 | 0.00257 | up |
| Myo19 | 100 | −1.14 | 0.453759578 | 0.0012 | 0.0211 | down |
| Efcab10 | 51 | 1.68 | 3.20427951 | 0.0000225 | 0.00104 | up |
| Sdc1 | 2010 | −1.7 | 0.307786103 | 3.61E−08 | 0.00000529 | down |
| Pxdn | 1350 | −1.27 | 0.414659773 | 0.000428 | 0.00989 | down |
| Pimreg | 30 | −1.86 | 0.275476279 | 0.001 | 0.0184 | down |
| Slc6a4 | 608 | −3.9 | 0.066985841 | 0.0000475 | 0.00186 | down |
| Doc2b | 81 | −1.29 | 0.408951029 | 0.000986 | 0.0182 | down |
| Nr1d1 | 565 | 1.21 | 2.313376368 | 0.0000535 | 0.00206 | up |
| Ngb | 159 | 1.09 | 2.128740365 | 0.00145 | 0.0237 | up |
| Bdkrb2 | 220 | −1.23 | 0.426317446 | 1.3E−09 | 0.000000306 | down |
| Arg2 | 151 | −1.25 | 0.420448208 | 0.000153 | 0.00458 | down |
| Pcnx | 3660 | −1.23 | 0.426317446 | 9.63E−08 | 0.0000118 | down |
| Hhipl1 | 348 | −1.39 | 0.381564802 | 5.22E−08 | 0.00000698 | down |
| Meg3 | 31500 | −1.38 | 0.384218795 | 0 | 3E−10 | down |
| Nxnl2 | 58 | 1.16 | 2.234574276 | 0.00204 | 0.0301 | up |
| Gadd45g | 963 | −1.17 | 0.444421341 | 0.000122 | 0.00385 | down |
| Ahrr | 40 | −1.01 | 0.496546248 | 0.00139 | 0.0231 | down |
| Crhbp | 38 | 1.67 | 3.182145935 | 0.00153 | 0.0246 | up |
| Rasgrf2 | 2820 | −1.74 | 0.299369676 | 0 | 5.4E−09 | down |
| Fst | 1030 | −2.68 | 0.156041319 | 3E−10 | 9.25E−08 | down |
| Stmn4 | 1660 | −1.53 | 0.346277367 | 0.000000521 | 0.000046 | down |
| Bmp1 | 880 | −1.14 | 0.453759578 | 0.0000668 | 0.00244 | down |
| Fyb | 172 | −1.24 | 0.423372656 | 0.00126 | 0.0218 | down |
| Mroh2b | 96 | 1.43 | 2.694467154 | 0.000202 | 0.00566 | up |
| Myo10 | 1900 | −1.68 | 0.312082637 | 2.6E−09 | 0.000000574 | down |
| Shisa9 | 93 | −3.28 | 0.102948877 | 1.89E−08 | 0.00000315 | down |
| Ly6h | 1360 | 1.01 | 2.0139111 | 0.0000317 | 0.00136 | up |
| Gpihbp1 | 65 | 2.64 | 6.233316637 | 0.000000806 | 0.0000657 | up |
| Ly6g | 90 | −2.82 | 0.141610486 | 0.000101 | 0.00334 | down |
| Snai2 | 42 | −1.56 | 0.339151082 | 0.000494 | 0.011 | down |
| Rfc4 | 70 | 1.05 | 2.070529848 | 0.001 | 0.0184 | up |
| Adamts1 | 491 | −1.09 | 0.469761375 | 0.000182 | 0.00521 | down |
| Stfa2 | 31 | −4.91 | 0.033261568 | 0.00365 | 0.0439 | down |
| Slc4a8 | 1390 | −1.68 | 0.312082637 | 0 | 1.45E−08 | down |
| Cdkn1a | 4350 | −1.28 | 0.411795509 | 0.000000158 | 0.000017 | down |
| Slc38a1 | 1570 | −1.01 | 0.496546248 | 2.51E−08 | 0.00000398 | down |
| Kcnk16 | 308 | −2.36 | 0.194791145 | 0.0000585 | 0.00221 | down |
| Celsr3 | 1250 | −1.03 | 0.489710149 | 0.000000286 | 0.000027 | down |
| C1qtnf12 | 372 | 1.03 | 2.042024251 | 2.94E−08 | 0.00000444 | up |
| Xdh | 969 | −2.04 | 0.243163737 | 2E−10 | 5.28E−08 | down |
| Rhoq | 6630 | −1.59 | 0.332171454 | 0 | 0 | down |
| Sox8 | 373 | 1.38 | 2.602683711 | 0.000365 | 0.0088 | up |
| Pdzph1 | 112 | 1.5 | 2.828427125 | 0.00308 | 0.0392 | up |
| Epb41l4a | 972 | −1.76 | 0.295248165 | 0 | 2.5E−09 | down |
| Grp | 44 | −3.14 | 0.113439894 | 0.0000617 | 0.0023 | down |
| Anxa1 | 936 | −2.09 | 0.234880687 | 0.00000257 | 0.000177 | down |
| Fam111a | 393 | −1.52 | 0.348685917 | 0 | 0 | down |
| Slc15a3 | 151 | −2.3 | 0.203063099 | 0.00000267 | 0.000182 | down |
| Lipk | 88 | −1.65 | 0.318640157 | 0.000000181 | 0.0000185 | down |
| Rin1 | 293 | −3.22 | 0.10732068 | 0 | 0 | down |
| Slc29a2 | 453 | −1.28 | 0.411795509 | 2E−10 | 6.32E−08 | down |
| Gal | 10200 | −3.54 | 0.085971364 | 3.8E−09 | 0.000000767 | down |
| Fosl1 | 82 | −1.76 | 0.295248165 | 7.31E−08 | 0.00000949 | down |
| Cyp26a1 | 94 | −2.96 | 0.128514228 | 1.7E−09 | 0.000000394 | down |
| Rbp4 | 48 | 3.03 | 8.168097006 | 0.00195 | 0.0291 | up |
| Gfra1 | 7620 | −1.22 | 0.429282718 | 4.01E−08 | 0.00000569 | down |
| Bhlhe22 | 34 | 1.84 | 3.580100284 | 0.00143 | 0.0235 | up |
| Arhgap19 | 957 | 1.04 | 2.056227653 | 0.00314 | 0.0398 | up |
| Hps1 | 345 | −1.05 | 0.482968164 | 0.0000178 | 0.000865 | down |
| Itga7 | 10600 | −1.55 | 0.341510064 | 0 | 0 | down |
| Adam8 | 1550 | −2.18 | 0.220675749 | 0 | 2.2E−09 | down |
| Podxl | 823 | −1.03 | 0.489710149 | 0.000197 | 0.00558 | down |
| Bach1 | 944 | −1.48 | 0.358488812 | 1.62E−08 | 0.00000272 | down |
| Pfkfb4 | 805 | −1.01 | 0.496546248 | 8.4E−09 | 0.00000157 | down |
| Col7a1 | 219 | −1.87 | 0.273573425 | 0 | 2.5E−09 | down |
| Dhtkd1 | 98 | −1.13 | 0.456915725 | 0.00118 | 0.0208 | down |
| Slc9a2 | 62 | 1.05 | 2.070529848 | 0.00366 | 0.044 | up |
| Il1r1 | 434 | −1.22 | 0.429282718 | 0.00000012 | 0.0000135 | down |
| Mstn | 38 | −1.75 | 0.297301779 | 0.00189 | 0.0285 | down |
| Bard1 | 47 | −1.25 | 0.420448208 | 0.000262 | 0.00694 | down |
| Des | 194 | 2.04 | 4.112455307 | 0.0000614 | 0.00229 | up |
| Ecel1 | 2060 | −2.51 | 0.175555609 | 0.00319 | 0.0403 | down |
| Efhd1 | 578 | 1.11 | 2.158456473 | 0.00246 | 0.0338 | up |
| Bok | 293 | 1.27 | 2.411615655 | 0.00000679 | 0.000406 | up |
| Dusp27 | 90 | −1.73 | 0.301451957 | 0.000226 | 0.00615 | down |
| Pou2f1 | 370 | −1.23 | 0.426317446 | 4.52E−08 | 0.00000617 | down |
| Cr2 | 44 | −3.18 | 0.110337875 | 3E−10 | 8.65E−08 | down |
| Atf3 | 6100 | −2.27 | 0.207329886 | 0.000645 | 0.0133 | down |
| Lamb3 | 72 | −1.99 | 0.251738888 | 0.0000127 | 0.000673 | down |
| Ddr2 | 485 | −1.04 | 0.486327474 | 0.000297 | 0.0076 | down |
| Hsd17b7 | 3390 | −1.51 | 0.351111219 | 0 | 0 | down |
| Acvr1c | 84 | −2.52 | 0.174342958 | 3.54E−08 | 0.00000526 | down |
| Acvr1 | 882 | −1.17 | 0.444421341 | 2E−10 | 6.83E−08 | down |
| Cybrd1 | 209 | −1.31 | 0.40332088 | 0.0000421 | 0.0017 | down |
| Fgf7 | 154 | 1.16 | 2.234574276 | 0.00282 | 0.0368 | up |
| Syt13 | 97 | 1.13 | 2.188587403 | 0.00401 | 0.0467 | up |
| Frmd5 | 121 | −1.21 | 0.432268616 | 0.00000115 | 0.0000891 | down |
| Chad1 | 485 | −1.78 | 0.291183397 | 0 | 0 | down |
| Car1 | 207 | −2.2 | 0.217637641 | 0.0000167 | 0.00082 | down |
| Car3 | 1320 | −1.91 | 0.266092546 | 0.000425 | 0.00983 | down |
| Mrgbp | 330 | −1.06 | 0.47963206 | 1.6E−09 | 0.000000373 | down |
| Procr | 204 | −1.64 | 0.320856474 | 0.00000263 | 0.00018 | down |
| Tnik | 3310 | −1.56 | 0.339151082 | 0 | 0.000000003 | down |
| Ptx3 | 78 | −1.7 | 0.307786103 | 0.000416 | 0.0097 | down |
| Olfml3 | 721 | 1.6 | 3.031433133 | 0.0000227 | 0.00105 | up |
| S100a11 | 2430 | −1.62 | 0.325335464 | 0.000000268 | 0.0000255 | down |
| Sprr2j-ps | 45 | −7.02 | 0.007704943 | 0.00000773 | 0.000451 | down |
| Vcam1 | 384 | 1.28 | 2.428389769 | 0.000297 | 0.0076 | up |
| Rnpc3 | 306 | −1.01 | 0.496546248 | 0.000041 | 0.00165 | down |
| Alpk1 | 232 | −1.11 | 0.463294031 | 0.00197 | 0.0292 | down |
| Nudt17 | 91 | −1.14 | 0.453759578 | 0.000258 | 0.00685 | down |
| Col24a1 | 762 | −1.45 | 0.366021424 | 9.89E−08 | 0.000012 | down |
| Mmp16 | 1110 | −2.81 | 0.142595464 | 0 | 0 | down |
| Clca1 | 33 | −3.11 | 0.115823508 | 0.000000517 | 0.0000459 | down |
| Wdr31 | 47 | 1 | 2 | 0.00446 | 0.05 | up |
| Tpm2 | 558 | 1.7 | 3.249009585 | 0.0000717 | 0.00256 | up |
| Plin2 | 3070 | −1.53 | 0.346277367 | 0 | 7.3E−09 | down |
| Pde4b | 655 | −1.26 | 0.41754396 | 0 | 1.36E−08 | down |
| Tnfrsf8 | 150 | −2.98 | 0.126744935 | 0.000000368 | 0.0000337 | down |
| Ephb2 | 215 | −1.16 | 0.447512535 | 0.00000506 | 0.000312 | down |
| Cyp4b1 | 363 | −2.33 | 0.198884121 | 0 | 5.5E−09 | down |
| Stil | 30 | −1.78 | 0.291183397 | 0.00162 | 0.0256 | down |
| Tinagl1 | 278 | 1.1 | 2.143546925 | 0.0000304 | 0.00132 | up |
| Sesn2 | 271 | −1.06 | 0.47963206 | 0.00000116 | 0.0000891 | down |
| Padi2 | 356 | 1.22 | 2.329467173 | 0.0013 | 0.0222 | up |
| Per3 | 400 | 1.14 | 2.203810232 | 0.000613 | 0.0129 | up |
| Rbp7 | 39 | 3.02 | 8.111675838 | 0.000181 | 0.00521 | up |
| Draxin | 446 | −2.22 | 0.214641359 | 0 | 2.2E−09 | down |
| Nppb | 381 | 1.28 | 2.428389769 | 0.00000272 | 0.000184 | up |
| Nsg1 | 3080 | −1.02 | 0.493116352 | 0.00000115 | 0.0000891 | down |
| Slc30a3 | 38 | 9.34 | 648.0673761 | 0.000558 | 0.0121 | up |
| Cckar | 1360 | −1.11 | 0.463294031 | 0.000006 | 0.000365 | down |
| Gnpda2 | 1230 | −1.11 | 0.463294031 | 1.26E−08 | 0.0000022 | down |
| Gabra4 | 41 | 1.25 | 2.37841423 | 0.003 | 0.0386 | up |
| Pxmp2 | 147 | 1.1 | 2.143546925 | 0.00000381 | 0.000247 | up |
| Tes | 436 | −1.09 | 0.469761375 | 0.000000177 | 0.0000182 | down |
| Rasl11a | 119 | −1.91 | 0.266092546 | 0.000000104 | 0.0000122 | down |
| Mtus2 | 1180 | −1.4 | 0.378929142 | 0 | 0 | down |
| Akr1b8 | 342 | −1.19 | 0.438302861 | 0.00000105 | 0.0000825 | down |
| Dusp11 | 1400 | −1.02 | 0.493116352 | 2.09E−08 | 0.00000338 | down |
| Gkn3 | 63 | 4.49 | 22.47111801 | 0.000000193 | 0.0000194 | up |
| Chl1 | 7010 | −2 | 0.25 | 0 | 0 | down |
| Aldh1l1 | 471 | −1.22 | 0.429282718 | 0.000000195 | 0.0000196 | down |
| Grip2 | 266 | −1.19 | 0.438302861 | 0 | 5E−10 | down |
| Mfap5 | 622 | 1.28 | 2.428389769 | 0.0014 | 0.0232 | up |
| Ptpro | 561 | −1.31 | 0.40332088 | 0.00000532 | 0.000328 | down |
| Bcat1 | 3050 | −1.08 | 0.473028823 | 4.26E−08 | 0.00000589 | down |
| Camk1 | 2580 | −1.1 | 0.466516496 | 0.000000303 | 0.0000283 | down |
| Ckm | 445 | 7.26 | 153.2772742 | 0.000111 | 0.00359 | up |
| Siglece | 53 | −1.8 | 0.287174589 | 0.00154 | 0.0247 | down |
| Apba2 | 2300 | −1.01 | 0.496546248 | 7.1E−09 | 0.00000135 | down |
| Tmc5 | 157 | −1.75 | 0.297301779 | 0.000000098 | 0.0000119 | down |
| Kif22 | 456 | −1.58 | 0.334481889 | 0.00000225 | 0.000157 | down |
| Zkscan2 | 158 | −1.02 | 0.493116352 | 0.0000538 | 0.00206 | down |
| Ptpn5 | 382 | −1.83 | 0.281264621 | 0 | 0 | down |
| Cpxm2 | 85 | 1.52 | 2.867910496 | 0.000487 | 0.0109 | up |
| Dnhd1 | 105 | −1.1 | 0.466516496 | 0.000643 | 0.0133 | down |
| Cckbr | 989 | −2.2 | 0.217637641 | 0.00234 | 0.0326 | down |
| Mrgprf | 127 | 1.38 | 2.602683711 | 0.00373 | 0.0446 | up |
| Fgf3 | 375 | −2.57 | 0.168404197 | 0.000875 | 0.0167 | down |
| Tnni2 | 242 | 5.45 | 43.71328822 | 0.00267 | 0.0356 | up |
| Magix | 60 | 1.49 | 2.808889751 | 0.000638 | 0.0133 | up |
| Chrdl1 | 177 | 1.19 | 2.281527432 | 0.00337 | 0.0415 | up |
| Slc7a3 | 100 | −1.21 | 0.432268616 | 0.000000916 | 0.000073 | down |
| Il2rg | 74 | −1.2 | 0.435275282 | 0.0037 | 0.0444 | down |
| Anxa10 | 363 | −4.4 | 0.047366143 | 0.0000876 | 0.00297 | down |
| Nkd1 | 393 | 1.26 | 2.394957409 | 0 | 1.8E−09 | up |
| Smad1 | 1380 | −1.18 | 0.441351498 | 0 | 0 | down |
| Slc12a3 | 176 | −1.95 | 0.258816231 | 0.00000228 | 0.000159 | down |
| Pllp | 632 | 1.64 | 3.116658319 | 0.00000298 | 0.000199 | up |
| Comp | 227 | 1.49 | 2.808889751 | 0.000693 | 0.014 | up |
| Oaf | 415 | 1.04 | 2.056227653 | 0.0000458 | 0.00181 | up |
| Cryab | 6370 | 1.07 | 2.099433367 | 0.000639 | 0.0133 | up |
| Tagln | 104 | 1.42 | 2.67585511 | 0.000472 | 0.0107 | up |
| Aqp9 | 48 | −2.45 | 0.183010712 | 0.000247 | 0.00664 | down |
| Gsta4 | 451 | 1.04 | 2.056227653 | 0.0000846 | 0.0029 | up |
| Mlip | 651 | 1.26 | 2.394957409 | 0.000165 | 0.00481 | up |
| Nktr | 1090 | −1.25 | 0.420448208 | 0.000963 | 0.0179 | down |
| Bfsp2 | 39 | 2.9 | 7.464263932 | 0.0000829 | 0.00285 | up |
| Fhl3 | 150 | 1.08 | 2.114036081 | 0.00256 | 0.0346 | up |
| Trib3 | 143 | −1.16 | 0.447512535 | 0.000000254 | 0.0000246 | down |
| Lmo7 | 1960 | −3.59 | 0.083042863 | 0 | 0 | down |
| Galnt9 | 1430 | −1.06 | 0.47963206 | 2.5E−09 | 0.000000558 | down |
| Chst2 | 3790 | −1.33 | 0.397768242 | 0 | 1E−10 | down |
| Frrs1 | 40 | −1.41 | 0.376311687 | 0.000374 | 0.00896 | down |
| Cryzl2 | 145 | 1.47 | 2.770218936 | 0.0000491 | 0.00192 | up |
| Mapkbp1 | 757 | −1.54 | 0.343885455 | 0 | 6E−10 | down |
| Coil | 498 | −1.07 | 0.476318999 | 3E−10 | 8.02E−08 | down |
| Ano7 | 149 | −2.07 | 0.2381595 | 0.00000026 | 0.0000249 | down |
| Scx | 332 | 1.12 | 2.173469725 | 0.000304 | 0.00773 | up |
| Emid1 | 406 | 1.78 | 3.434261746 | 0.000178 | 0.00515 | up |
| Loxl2 | 724 | −1.82 | 0.283220971 | 0.0000005 | 0.0000446 | down |
| Igsf9b | 1170 | −1.55 | 0.341510064 | 3E−10 | 8.02E−08 | down |
| Cd207 | 37 | −2.84 | 0.139660892 | 0.0000757 | 0.00268 | down |
| Cpne7 | 51 | 1.79 | 3.458148925 | 0.0014 | 0.0232 | up |
| Tet3 | 877 | −1.1 | 0.466516496 | 0.00000602 | 0.000365 | down |
| Tbxa2r | 117 | 1.37 | 2.584705661 | 0.000471 | 0.0107 | up |
| Tpbg | 65 | −2.66 | 0.158219574 | 0.000000365 | 0.0000336 | down |
| Ccl12 | 129 | −2.21 | 0.216134308 | 0.00209 | 0.0305 | down |
| Kcnh4 | 32 | −3.16 | 0.111878134 | 0.0000297 | 0.00129 | down |
| Nfkbiz | 135 | −1.38 | 0.384218795 | 0.000124 | 0.0039 | down |
| Sstr1 | 34 | −1.59 | 0.332171454 | 0.00131 | 0.0222 | down |
| Plk5 | 326 | 1.36 | 2.566851795 | 0.00077 | 0.0152 | up |
| Leng8 | 4650 | −1.03 | 0.489710149 | 0.000207 | 0.00576 | down |
| Chrna5 | 92 | −3.26 | 0.10438599 | 6.19E−08 | 0.00000818 | down |
| Sbno2 | 2640 | −1.6 | 0.329876978 | 0 | 0 | down |
| Nme5 | 95 | 1.18 | 2.265767771 | 0.0000994 | 0.00329 | up |
| Zfp57 | 93 | −1.46 | 0.363493129 | 0.000000829 | 0.0000672 | down |
| Gadd45a | 2640 | −1.74 | 0.299369676 | 2.7E−09 | 0.000000582 | down |
| Mag | 593 | 1.24 | 2.361985323 | 0.00033 | 0.0082 | up |
| Proser3 | 113 | −1.06 | 0.47963206 | 0.0000963 | 0.00321 | down |
| Arhgap33 | 1630 | −1.43 | 0.371130893 | 0 | 5.7E−09 | down |
| Inhbb | 790 | −3.08 | 0.118257206 | 0 | 0 | down |
| Galnt6 | 308 | −1.64 | 0.320856474 | 0.00000205 | 0.000146 | down |
| Serpine1 | 141 | −2.21 | 0.216134308 | 0.000135 | 0.00412 | down |
| Vgf | 11900 | −1.15 | 0.450625231 | 0.000129 | 0.00401 | down |
| Vash2 | 231 | −1.13 | 0.456915725 | 0.00000232 | 0.000161 | down |
| Cdkn1c | 186 | 1.6 | 3.031433133 | 0.000282 | 0.00735 | up |
| Tecta | 662 | −1.46 | 0.363493129 | 1.95E−08 | 0.00000322 | down |
| Egr2 | 404 | 1.27 | 2.411615655 | 0.00289 | 0.0375 | up |
| Smim3 | 1270 | −2.08 | 0.236514412 | 0 | 7.9E−09 | down |
| Hspb2 | 135 | 1.54 | 2.907945035 | 0.0000541 | 0.00207 | up |
| Prdm1 | 76 | −2.89 | 0.13490353 | 1.5E−09 | 0.000000354 | down |
| Slc22a23 | 2460 | −1.08 | 0.473028823 | 0.00000304 | 0.000202 | down |
| Egr1 | 1910 | 2 | 4 | 0.00159 | 0.0254 | up |
| 1700003F12Rik | 93 | −2.09 | 0.234880687 | 0.0000162 | 0.000809 | down |
| Ciart | 101 | 1.45 | 2.732080514 | 0.000343 | 0.0084 | up |
| Cyp24a1 | 61 | −4.43 | 0.046391362 | 1.7E−09 | 0.000000386 | down |
| Atp5l | 57 | −1.24 | 0.423372656 | 0.00143 | 0.0235 | down |
| Irs2 | 3450 | −1.46 | 0.363493129 | 5E−10 | 0.000000119 | down |
| Prc1 | 214 | −1.9 | 0.267943366 | 0.000000121 | 0.0000135 | down |
| Srrm2 | 8970 | −1.06 | 0.47963206 | 0.000138 | 0.0042 | down |
| Rnf122 | 687 | −1.38 | 0.384218795 | 1E−10 | 2.21E−08 | down |
| Ajap1 | 65 | −4.85 | 0.034674046 | 0 | 8.3E−09 | down |
| Thap3 | 226 | 1.01 | 2.0139111 | 0.00000282 | 0.00019 | up |
| Lrrc17 | 144 | 1.28 | 2.428389769 | 0.0000116 | 0.000624 | up |
| Cited2 | 824 | −1.52 | 0.348685917 | 0 | 0 | down |
| Cacna2d1 | 11300 | −1.47 | 0.360982299 | 5E−10 | 0.000000119 | down |
| Fmo2 | 409 | 1.54 | 2.907945035 | 0.0000287 | 0.00126 | up |
| Plekha4 | 6180 | 1.08 | 2.114036081 | 0.00287 | 0.0372 | up |
| Gstm2 | 225 | 1.17 | 2.250116969 | 0.0000793 | 0.00275 | up |
| Ildr2 | 433 | −1.41 | 0.376311687 | 0.00000151 | 0.000113 | down |
| Clcf1 | 88 | −1.39 | 0.381564802 | 0.00203 | 0.0299 | down |
| Myh6 | 130 | −2.08 | 0.236514412 | 0.000796 | 0.0155 | down |
| Dlk1 | 56 | 1.97 | 3.91768119 | 0.00334 | 0.0413 | up |
| Tmco4 | 168 | 1.3 | 2.462288827 | 0.000222 | 0.00608 | up |
| Hectd2 | 71 | −1.74 | 0.299369676 | 0.0000322 | 0.00137 | down |
| Chrnb1 | 64 | −1.25 | 0.420448208 | 0.0000128 | 0.000673 | down |
| Pla2g5 | 42 | 1.73 | 3.317278183 | 0.000771 | 0.0152 | up |
| Cldn5 | 714 | 1.33 | 2.514026749 | 0.000309 | 0.00785 | up |
| Tmtc4 | 1240 | −1.19 | 0.438302861 | 3.9E−09 | 0.000000779 | down |
| Mbp | 55100 | 1.51 | 2.848100391 | 0.00095 | 0.0178 | up |
| Zmym6 | 326 | −1.02 | 0.493116352 | 0.000148 | 0.00445 | down |
| Reln | 1100 | 1.1 | 2.143546925 | 0.00303 | 0.0387 | up |
| Fam227a | 51 | −1.08 | 0.473028823 | 0.000101 | 0.00332 | down |
| Mapk6 | 1060 | −1.09 | 0.469761375 | 5E−10 | 0.000000139 | down |
| Id1 | 521 | 1.59 | 3.010493495 | 0.000155 | 0.00463 | up |
| Gpr151 | 1430 | −3.34 | 0.098755164 | 0.00000136 | 0.000103 | down |
| Fgf11 | 1190 | −1.1 | 0.466516496 | 0.0000028 | 0.000188 | down |
| Tuba1c | 238 | −1.03 | 0.489710149 | 0.0000274 | 0.00122 | down |
| Olfr78 | 51 | −1.23 | 0.426317446 | 0.0013 | 0.0222 | down |
| Zbtb37 | 187 | −1.36 | 0.38958229 | 0.000381 | 0.00905 | down |
| Mdga1 | 1950 | −1.48 | 0.358488812 | 6.8E−09 | 0.00000131 | down |
| Clec4a3 | 35 | −1.69 | 0.309926925 | 0.00202 | 0.0299 | down |
| Ncmap | 2330 | 1.6 | 3.031433133 | 0.000027 | 0.0012 | up |
| Adgrd1 | 887 | −2.06 | 0.23981603 | 4E−10 | 9.38E−08 | down |
| Cdkn2a | 324 | 2.2 | 4.59479342 | 0.00000597 | 0.000364 | up |
| Snhg11 | 19200 | −1.17 | 0.444421341 | 3.6E−09 | 0.000000743 | down |
| Tnfaip8l1 | 105 | 1.75 | 3.363585661 | 0.00117 | 0.0207 | up |
| Lncpint | 35 | −1.38 | 0.384218795 | 0.00159 | 0.0253 | down |
| Liph | 90 | −1.56 | 0.339151082 | 0.000185 | 0.00529 | down |
| Pard6b | 161 | −1.25 | 0.420448208 | 1E−10 | 2.17E−08 | down |
| Plppr4 | 360 | −1.68 | 0.312082637 | 3E−10 | 8.02E−08 | down |
| Slc38a6 | 206 | −1.19 | 0.438302861 | 1E−10 | 0.00000003 | down |
| Serpinb1a | 4180 | −1.09 | 0.469761375 | 0.0000327 | 0.00139 | down |
| Pcdhb8 | 30 | −1.44 | 0.368567304 | 0.00123 | 0.0214 | down |
| D830044D21Rik | 34 | −1.55 | 0.341510064 | 0.00235 | 0.0327 | down |
| Chrm3 | 67 | −1.01 | 0.496546248 | 0.000684 | 0.0139 | down |
| Cd109 | 244 | −1.17 | 0.444421341 | 0.0000342 | 0.00144 | down |
| Plaur | 391 | −1.05 | 0.482968164 | 0.000011 | 0.000597 | down |
| Fam178b | 905 | 1.42 | 2.67585511 | 0.0000625 | 0.00232 | up |
| Lrrc75a | 263 | 1.24 | 2.361985323 | 0.00000465 | 0.000292 | up |
| Nexmif | 109 | −1 | 0.5 | 0.00223 | 0.0317 | down |
| Tifa | 956 | −1.33 | 0.397768242 | 0.000000168 | 0.0000177 | down |
| Fam46b | 44 | 1.52 | 2.867910496 | 0.0000761 | 0.00268 | up |
| Prr15l | 57 | 1.24 | 2.361985323 | 0.0016 | 0.0254 | up |
| Chst9 | 30 | 1.72 | 3.294364069 | 0.000639 | 0.0133 | up |
| Gap43 | 15000 | −1.19 | 0.438302861 | 0.000000868 | 0.0000698 | down |
| Kcne4 | 31 | 1.31 | 2.4794154 | 0.00156 | 0.0249 | up |
| Tgif1 | 251 | −1.08 | 0.473028823 | 0.00288 | 0.0374 | down |
| Flrt2 | 441 | −1.47 | 0.360982299 | 1.29E−08 | 0.00000221 | down |
| Gjb1 | 379 | 1.16 | 2.234574276 | 0.00188 | 0.0284 | up |
| 8430408G22Rik | 130 | 1.13 | 2.188587403 | 0.000318 | 0.008 | up |
| Nrip1 | 2100 | −1.29 | 0.408951029 | 5E−10 | 0.000000139 | down |
| Fndc9 | 53 | −2.86 | 0.137738139 | 0.000000021 | 0.00000338 | down |
| Pthlh | 129 | −1.04 | 0.486327474 | 0.00327 | 0.0407 | down |
| Ccr2 | 98 | −1.41 | 0.376311687 | 0.00108 | 0.0194 | down |
| Adamts16 | 55 | −1.61 | 0.327598351 | 0.00279 | 0.0366 | down |
| Crh | 322 | −4.99 | 0.031467361 | 8.36E−08 | 0.0000105 | down |
| Rasd1 | 116 | 1.88 | 3.680750602 | 0.00000557 | 0.000341 | up |
| Grem2 | 199 | −1.74 | 0.299369676 | 8.5E−09 | 0.00000158 | down |
| Vwa3b | 35 | 1.5 | 2.828427125 | 0.00345 | 0.0422 | up |
| Mchr1 | 252 | −1.18 | 0.441351498 | 0.0000967 | 0.00322 | down |
| Prag1 | 507 | −1.39 | 0.381564802 | 0.0000269 | 0.0012 | down |
| Abca12 | 57 | −2.51 | 0.175555609 | 0.00000019 | 0.0000192 | down |
| Neto1 | 1000 | −2.17 | 0.22221067 | 0 | 0 | down |
| Sprr1a | 40500 | −3.4 | 0.094732285 | 0.00325 | 0.0406 | down |
| Prokr2 | 856 | −2.16 | 0.223756268 | 0 | 0 | down |
| Arhgap42 | 646 | −1.06 | 0.47963206 | 7E−10 | 0.000000177 | down |
| Trim15 | 76 | −3.12 | 0.115023456 | 2.4E−09 | 0.000000541 | down |
| Htr1f | 124 | −1.11 | 0.463294031 | 0.0000453 | 0.0018 | down |
| Serpinb1b | 325 | 1.47 | 2.770218936 | 0 | 5E−10 | up |
| Flrt3 | 2630 | −2.08 | 0.236514412 | 0 | 8.5E−09 | down |
| Pcdhb11 | 44 | −1.65 | 0.318640157 | 0.0000106 | 0.000586 | down |
| Kctd19 | 70 | 1.34 | 2.531513188 | 0.0022 | 0.0315 | up |
| AU021092 | 32 | 1.65 | 3.138336392 | 0.00108 | 0.0194 | up |
| Kcnf1 | 160 | 1.15 | 2.219138944 | 0.000363 | 0.00878 | up |
| Prdm9 | 102 | −1.57 | 0.336808394 | 0.0000259 | 0.00117 | down |
| Igfn1 | 176 | −3.53 | 0.086569342 | 0.000252 | 0.00673 | down |
| Slc6a7 | 32 | 2.34 | 5.063026376 | 0.000699 | 0.0141 | up |
| Cx3cr1 | 262 | −1.99 | 0.251738888 | 0.000165 | 0.00482 | down |
| Tchh | 60 | −1.62 | 0.325335464 | 0.000326 | 0.00816 | down |
| Epha3 | 87 | −2.11 | 0.231647015 | 0.000000294 | 0.0000276 | down |
| Nav2 | 897 | −1.56 | 0.339151082 | 0.000000261 | 0.0000249 | down |
| Hist1h1d | 98 | −1.7 | 0.307786103 | 0.000000596 | 0.0000516 | down |
| Pcdh15 | 71 | −1.04 | 0.486327474 | 0.0000808 | 0.0028 | down |
| Glis3 | 197 | −2.18 | 0.220675749 | 0.00000154 | 0.000115 | down |
| Socs3 | 414 | −1.13 | 0.456915725 | 0.00000268 | 0.000182 | down |
| Prx | 7710 | 1.54 | 2.907945035 | 0.00162 | 0.0256 | up |
| Styx | 74 | −1.32 | 0.400534939 | 0.000331 | 0.0082 | down |
| Aldh1a1 | 359 | 1.28 | 2.428389769 | 0.0000353 | 0.00147 | up |
| H2-T24 | 141 | −1.6 | 0.329876978 | 0.0000292 | 0.00128 | down |
| Adm2 | 47 | −2.85 | 0.138696184 | 0.00000842 | 0.000482 | down |
| Ceacam10 | 236 | 1.32 | 2.496661098 | 0.00126 | 0.0218 | up |
| 2900041M22Rik | 73 | 1.05 | 2.070529848 | 0.00296 | 0.0382 | up |
| Oscar | 30 | −1.08 | 0.473028823 | 0.00274 | 0.0362 | down |
| Gabra5 | 271 | −1.02 | 0.493116352 | 0.0000572 | 0.00217 | down |
| Arntl | 116 | −1.61 | 0.327598351 | 0.0000386 | 0.00158 | down |
| Klf2 | 593 | 1.4 | 2.639015822 | 0.000793 | 0.0155 | up |
| C1ra | 74 | 1.66 | 3.160165247 | 0.000645 | 0.0133 | up |
| Soga1 | 394 | −1.2 | 0.435275282 | 0.00312 | 0.0397 | down |
| Mettl4 | 111 | −1.01 | 0.496546248 | 0.000342 | 0.00839 | down |
| Myh1 | 195 | 4.65 | 25.10669113 | 0.00131 | 0.0222 | up |
| Mpz | 71300 | 1.28 | 2.428389769 | 0.000528 | 0.0115 | up |
| Capg | 954 | 1.11 | 2.158456473 | 0.0000184 | 0.000883 | up |
| Nfil3 | 373 | −1.52 | 0.348685917 | 2.1E−09 | 0.000000482 | down |
| Myh4 | 508 | 7.68 | 205.0738887 | 0.00124 | 0.0215 | up |
| Scn3a | 113 | −1.1 | 0.466516496 | 0.000245 | 0.00659 | down |
| Mettl7a3 | 1090 | −1.18 | 0.441351498 | 1.55E−08 | 0.00000262 | down |
| Sez6l | 4370 | −2.5 | 0.176776695 | 0 | 3E−10 | down |
| Pde1a | 581 | −1.56 | 0.339151082 | 0.000000157 | 0.000017 | down |
| Reg1 | 48 | −5.23 | 0.02664484 | 0.00223 | 0.0317 | down |
| Dbp | 593 | 2.13 | 4.377174805 | 9.43E−08 | 0.0000117 | up |
| Tmem40 | 91 | 1.15 | 2.219138944 | 0.00143 | 0.0235 | up |
| Plekhh1 | 600 | −1.09 | 0.469761375 | 0.000643 | 0.0133 | down |
| Mcpt4 | 105 | 2.32 | 4.993322196 | 0.00102 | 0.0187 | up |
| Rps7 | 907 | 1.05 | 2.070529848 | 0.000000101 | 0.0000121 | up |
| Tnnt3 | 499 | 6.42 | 85.62736351 | 0.000392 | 0.00925 | up |
| Ppef1 | 3240 | −1.07 | 0.476318999 | 1E−10 | 2.67E−08 | down |
| Cys1 | 306 | 1.42 | 2.67585511 | 2E−10 | 7.19E−08 | up |
| Phactr2 | 4850 | −1.17 | 0.444421341 | 0.000000417 | 0.0000378 | down |
| Sox7 | 149 | −2.09 | 0.234880687 | 0 | 1.8E−09 | down |
| Sox11 | 1840 | −2.17 | 0.22221067 | 9.8E−09 | 0.00000176 | down |
| Srrm4 | 837 | −1.44 | 0.368567304 | 0.000000243 | 0.0000237 | down |
| Klk8 | 72 | 1.79 | 3.458148925 | 0.000218 | 0.00601 | up |
| Cldn19 | 1840 | 1.54 | 2.907945035 | 0.000102 | 0.00334 | up |
| Fxyd6 | 3290 | 1 | 2 | 0.0000582 | 0.0022 | up |
| Syngap1 | 986 | −1.31 | 0.40332088 | 1E−10 | 0.00000003 | down |
| Myl9 | 613 | 1.46 | 2.751083636 | 0.00000499 | 0.000309 | up |
| Flnc | 785 | −1.71 | 0.305660069 | 0.00000688 | 0.000409 | down |
| Trp53i11 | 932 | −2.61 | 0.163799175 | 1E−10 | 0.000000034 | down |
| Gm5152 | 64 | −6.16 | 0.013984767 | 1.18E−08 | 0.00000209 | down |
| Slfn9 | 294 | −2.66 | 0.158219574 | 0 | 1.7E−09 | down |
| Tmem132b | 213 | −1.13 | 0.456915725 | 0.000172 | 0.00499 | down |
| Gm12966 | 33 | −1.17 | 0.444421341 | 0.000378 | 0.00901 | down |
| Tia1 | 633 | −1.31 | 0.40332088 | 0 | 0 | down |
| Reg3b | 31 | −5.61 | 0.020474897 | 0.00321 | 0.0404 | down |
| Klf12 | 152 | −1.44 | 0.368567304 | 0.000302 | 0.00769 | down |
| 5930412G12Rik | 71 | −1.03 | 0.489710149 | 0.00169 | 0.0263 | down |
| Slfn10-ps | 41 | −3.37 | 0.096722812 | 0.000112 | 0.00361 | down |
| Serpina1e | 82 | 1.19 | 2.281527432 | 0.00408 | 0.0472 | up |
| 9030624G23Rik | 49 | −1.46 | 0.363493129 | 0.00068 | 0.0138 | down |
| Sh2d1b2 | 53 | −4.45 | 0.045752678 | 0.000000393 | 0.0000359 | down |
| Dok6 | 68 | −1.7 | 0.307786103 | 0.00186 | 0.0282 | down |
| Papp a2 | 630 | −1.13 | 0.456915725 | 0.00105 | 0.019 | down |
| Tmem88b | 528 | −2.79 | 0.144586023 | 0 | 0 | down |
| Fam196a | 202 | −2.06 | 0.23981603 | 7.8E−09 | 0.00000147 | down |
| 4632427E13Rik | 36 | −1.31 | 0.40332088 | 0.00281 | 0.0367 | down |
| Plekhf1 | 188 | 1.43 | 2.694467154 | 0.000149 | 0.00445 | up |
| Cox7a1 | 98 | 1.11 | 2.158456473 | 0.00126 | 0.0217 | up |
| Mex3a | 68 | −1.5 | 0.353553391 | 0.000203 | 0.00567 | down |
| Gm10800 | 5200 | 6.38 | 83.28587875 | 0.000681 | 0.0138 | up |
| Ccdc162 | 105 | −1.07 | 0.476318999 | 0.0015 | 0.0242 | down |
| Scn2a | 279 | −1.16 | 0.447512535 | 0 | 1.47E−08 | down |
| Wfdc3 | 180 | −2.01 | 0.248273124 | 0.00269 | 0.0357 | down |
| Fyb2 | 93 | −1.14 | 0.453759578 | 0.000378 | 0.00901 | down |
| 5830417I10Rik | 103 | −1.38 | 0.384218795 | 0.000000794 | 0.0000655 | down |
| Ccr5 | 76 | −2.1 | 0.233258248 | 0.00000966 | 0.000543 | down |
| St6galnac4 | 471 | −1.06 | 0.47963206 | 9.4E−09 | 0.00000171 | down |
| Spaca6 | 261 | −1.01 | 0.496546248 | 0.000127 | 0.00396 | down |
| Hist2h3c2 | 105 | −1.31 | 0.40332088 | 0.000544 | 0.0118 | down |
| Bambi-ps1 | 34 | −2.24 | 0.211686328 | 0.0000101 | 0.000561 | down |
| Gm16185 | 55 | −2.38 | 0.192109398 | 0.0000154 | 0.000777 | down |
| Abhd11os | 98 | 1.6 | 3.031433133 | 0.000000156 | 0.0000169 | up |
| Ril1 | 105 | −1.04 | 0.486327474 | 0.0000441 | 0.00176 | down |
| Gm13571 | 31 | −3.56 | 0.08478777 | 0.000114 | 0.00366 | down |
| Ftx | 133 | −1.14 | 0.453759578 | 0.000122 | 0.00384 | down |
| Gm13446 | 74 | 1.12 | 2.173469725 | 0.00106 | 0.0191 | up |
| Gm15500 | 63 | 1.07 | 2.099433367 | 0.000887 | 0.0168 | up |
| A730081D07Rik | 31 | −1.18 | 0.441351498 | 0.00274 | 0.0362 | down |
| Gm15751 | 118 | −1.21 | 0.432268616 | 0.00247 | 0.0339 | down |
| Tbx3os2 | 166 | 1.09 | 2.128740365 | 0.000637 | 0.0133 | up |
| Hopxos | 30 | 1.33 | 2.514026749 | 0.00319 | 0.0403 | up |
| 4732440D04Rik | 60 | −1.42 | 0.373712312 | 0.00000189 | 0.000137 | down |
| Pou3f1 | 432 | 2.27 | 4.823231311 | 2.01E−08 | 0.00000329 | up |
| Gm38399 | 128 | −2.14 | 0.226879789 | 0.0000782 | 0.00273 | down |
| Gypc | 313 | 1.1 | 2.143546925 | 0.000418 | 0.00972 | up |
| Apold1 | 45 | 1.23 | 2.345669898 | 0.000743 | 0.0148 | up |
| Lrrc32 | 123 | 1.76 | 3.386981249 | 0.00000432 | 0.000275 | up |
| 9130204L05Rik | 65 | 2.08 | 4.228072162 | 0.00218 | 0.0313 | up |
| Malat1 | 11700 | −1.76 | 0.295248165 | 0.000362 | 0.00878 | down |
| Methig1 | 30 | −1.74 | 0.299369676 | 0.00377 | 0.0449 | down |
| Gm9866 | 385 | −1.06 | 0.47963206 | 0.0000207 | 0.000971 | down |
| Gm38394 | 58 | −1.83 | 0.281264621 | 0.000783 | 0.0153 | down |
| Gm21781 | 145 | −1.29 | 0.408951029 | 0.0000691 | 0.00249 | down |
| Gm9821 | 56 | −1.76 | 0.295248165 | 0.000577 | 0.0123 | down |
| A630023A22Rik | 47 | −3.55 | 0.085377516 | 0.000000169 | 0.0000178 | down |
| Gm10717 | 112 | 26.1 | 71925499.54 | 0.0000037 | 0.000242 | up |
| Gm26788 | 30 | −2.31 | 0.20166044 | 0.000572 | 0.0123 | down |
| Gm2694 | 65 | 1.03 | 2.042024251 | 0.00164 | 0.0258 | up |
| Gm17275 | 44 | −1.45 | 0.366021424 | 0.00139 | 0.0231 | down |
| Gm16793 | 38 | −1.33 | 0.397768242 | 0.000973 | 0.018 | down |
| Gm26783 | 30 | 2.29 | 4.890561111 | 0.000432 | 0.00993 | up |
| Rian | 7510 | −1.01 | 0.496546248 | 0 | 6E−10 | down |
| Mir124a-1hg | 1320 | −1.05 | 0.482968164 | 0.000000113 | 0.0000129 | down |
| 9530059O14Rik | 87 | −1.27 | 0.414659773 | 0.0000122 | 0.000651 | down |
| 9630001P10Rik | 38 | −1.58 | 0.334481889 | 0.000481 | 0.0108 | down |
| Gm26917 | 4660 | −1.44 | 0.368567304 | 0.00372 | 0.0445 | down |
| Gm26945 | 33 | −2.52 | 0.174342958 | 0.00118 | 0.0208 | down |
| Gm29374 | 201 | −3.96 | 0.064257114 | 0 | 8E−10 | down |
| Gm4208 | 140 | −3.24 | 0.105843164 | 0.000000336 | 0.0000311 | down |
| Gm28875 | 190 | 1.66 | 3.160165247 | 0.0000132 | 0.00069 | up |
| Gm37233 | 30 | −2.18 | 0.220675749 | 0.00117 | 0.0207 | down |
| Pcdhgb2 | 68 | −1.13 | 0.456915725 | 0.0034 | 0.0418 | down |
| Gm7694 | 195 | −1.48 | 0.358488812 | 0.00000016 | 0.0000171 | down |
| Pcdhgb1 | 64 | −1.95 | 0.258816231 | 0.00147 | 0.0239 | down |
| Gm37092 | 124 | −1.09 | 0.469761375 | 0.0000241 | 0.0011 | down |
| Pcdha12 | 57 | −1.28 | 0.411795509 | 0.00265 | 0.0354 | down |
| 2210017I01Rik | 37 | −1.72 | 0.303548721 | 0.0042 | 0.048 | down |
| Pcdhgb4 | 40 | −1.58 | 0.334481889 | 0.000315 | 0.00794 | down |
| Gm38020 | 34 | −2.52 | 0.174342958 | 0.000457 | 0.0104 | down |
| Gm20045 | 51 | −1.08 | 0.473028823 | 0.00246 | 0.0338 | down |
| Gm31831 | 31 | −6.54 | 0.01074642 | 0.0000557 | 0.00213 | down |
| Gm6204 | 57 | 1.33 | 2.514026749 | 0.000694 | 0.014 | up |
| 4932422M17Rik | 56 | −1.29 | 0.408951029 | 0.000482 | 0.0108 | down |
| AI506816 | 141 | −1.11 | 0.463294031 | 0.000000962 | 0.0000761 | down |
| Gm42621 | 60 | 1.44 | 2.713208655 | 0.00162 | 0.0256 | up |
| Gm42664 | 32 | −2.43 | 0.185565446 | 0.00263 | 0.0353 | down |
| Gm43843 | 43 | −1.86 | 0.275476279 | 0.00196 | 0.0292 | down |
| Gm2762 | 40 | −2.36 | 0.194791145 | 9.62E−08 | 0.0000118 | down |
| Gm34583 | 256 | −2.17 | 0.22221067 | 0.000105 | 0.00344 | down |
| Gm42788 | 37 | 1.61 | 3.052518418 | 0.00115 | 0.0204 | up |
| Gm43980 | 91 | −1.85 | 0.277392368 | 0.000407 | 0.00952 | down |
| Gm44168 | 32 | −1.44 | 0.368567304 | 0.00439 | 0.0495 | down |
| Gm5886 | 39 | 1.42 | 2.67585511 | 0.00439 | 0.0495 | up |
| Gm7972 | 117 | −3.4 | 0.094732285 | 6.3E−09 | 0.00000123 | down |
| Gm45867 | 38 | −1.46 | 0.363493129 | 0.0013 | 0.0221 | down |
| Gm30085 | 53 | −2.14 | 0.226879789 | 0.000127 | 0.00396 | down |
| C78859 | 2090 | −1.49 | 0.356012549 | 0 | 6.9E−09 | down |
| Gm47133 | 41 | −2.34 | 0.197510328 | 0.0000948 | 0.00318 | down |
| Btbd8 | 825 | −1.01 | 0.496546248 | 0.000000147 | 0.0000161 | down |
| AC160637.1 | 161 | −1.73 | 0.301451957 | 0.00000151 | 0.000113 | down |
| Gm47593 | 147 | −2.25 | 0.210224104 | 0.000000617 | 0.0000532 | down |
| Gm47595 | 106 | −2.3 | 0.203063099 | 0.000000729 | 0.0000611 | down |
| Gm35721 | 90 | 1.04 | 2.056227653 | 0.00103 | 0.0187 | up |
| Gm47903 | 47 | −4.77 | 0.036651092 | 0.00000813 | 0.00047 | down |
| E430024I08Rik | 75 | −1.18 | 0.441351498 | 0.00125 | 0.0216 | down |
| Tubb2a-ps2 | 47 | −1.08 | 0.473028823 | 0.004 | 0.0466 | down |
| CT030170.4 | 75 | −1.27 | 0.414659773 | 0.00000239 | 0.000165 | down |
| Gm48239 | 49 | −2.09 | 0.234880687 | 0.000117 | 0.00374 | down |

### Example 3

### Identifying Drugs for Treating Pain

In order to identify new drugs for the treatment of pain, the present inventors sought to identify drug leads that might mimic or target the importin α3-c-Fos pathway. To this end the differentially expressed gene-sets from importin α3 null DRG were used query the Connectivity Map (CMap), a database of transcriptional signatures of numerous approved drugs and drug leads^{22,23}. This analysis revealed several compounds with CMap scores consistent with similar transcriptional effects as the importin α3 knockout (Table 3 hereinabove). Interestingly, gabapentin was not highly ranked in this CMap analysis, indicating that if the newly identified compounds indeed affect pain, their mode of action should likely be distinct from that of gabapentin.

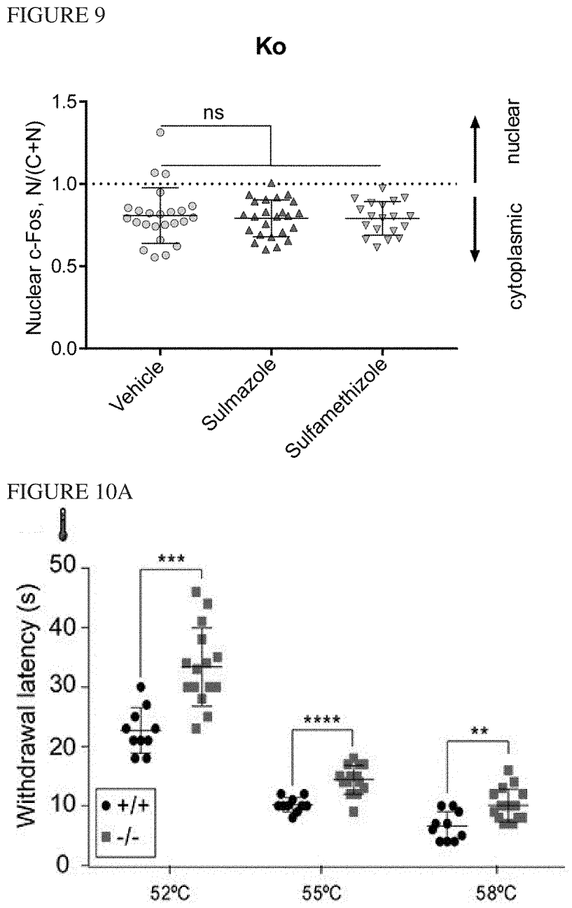

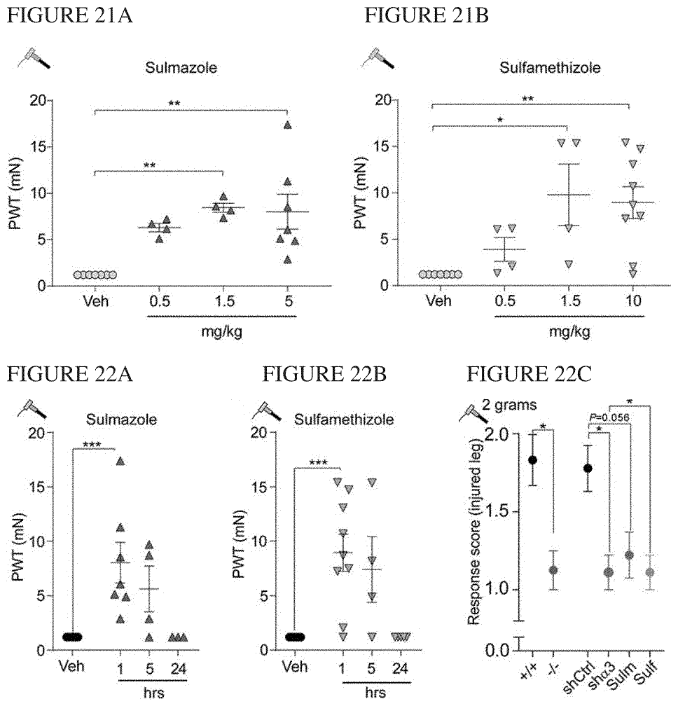

Two compounds from the topmost ranked subset were selected for further analysis, namely sulmazole, a cardiotonic agent, and sulfamethizole, an antibiotic. In a first test for reduced responsiveness to noxious heat both sulmazole and sulfamethizole showed efficacy in the assay (FIG. 8A). Strikingly, both sulmazole and sulfamethizole also provided time and dose-dependent relief in the SNI model of neuropathic pain when tested by intraperitoneal injection at both early and late stages of chronic pain (FIGS. 8B, 21A-B and 22A-B). The effects of the drugs on response of SNI injured animals to a noxious mechanical stimulus was also determined. Both drugs provided relief in this assay that was comparable to that provided by knockout or knockdown of importin α3 (FIG. 22C). Following, the impact of both compounds on the subcellular localization of c-Fos in cultured DRG neurons was assessed. Both sulmazole and sulfamethizole significantly reduced c-Fos nuclear accumulation in wild type neurons (FIGS. 8C-D). Notably, neither drug had any further effect on c-Fos nuclear accumulation in importin α3 null neurons, beyond that already induced by the knockout (FIG. 9). Thus, drugs mimicking the transcriptional signature of loss of the importin α3 can phenocopy both the analgesic and c-Fos localization effects observed in the importin α3 mutant animals.

### Example 4

### Inhibition of Importin α3 at the Protein Level Reduces Pain Sensitivity to Noxious Heat, Chemically-Induced and Neuropathic Pain

Several importin α 3 inhibitory agents are designed, namely:

1) A dominant negative peptide comprising c-Fos NLS sequence: QLSPEEEEKRRIRRERNKMAAAKCR (SEQ ID NO: 20), or a portion thereof;

2) A dominant negative peptide comprising c-Jun NLS sequence: RIKAERKRMRNRIAASKCRKRKLERIARLEEKVKTLKAQNSE (SEQ ID NO: 21) or a portion thereof;

3) An antibody raised against importin α3 that e.g. prevents interactions of c-Fos and/or c-Jun with importin α 3.

Following, these agents are tested for their effect on sensitivity to noxious heat, chemically-induced and neuropathic pain using the methods described hereinabove.

According to specific embodiments, a nucleic acid encoding the agent is introduced into the model animal.

To this end, a nucleic acid sequence encoding the agent is cloned into an AAV vector, using serotypes appropriate for the specific objective. Sensory neurons are transduced by AAV serotypes 9 or PhP.s, as described hereinabove. For all constructs, a titer in the range of 10¹²-10¹³ viral genomes/ml can be used for intrathecal injections into the lumbar spinal cord.

Alternatively, or additionally, according to specific embodiments, the agent is produced synthetically (e.g. using solid phase) or recombinantly, purified and administered to the lumbar spinal cord or DRGs by e.g. minipumps. In a specific variation of this approach the agent may be fused or mixed with membrane penetrating agents such as the Transactivating transcriptional activator (TAT) peptide, Antennapedia, penetratin, and other agents of this class.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

1. Miyamoto et al. Importin alpha: a key molecule in nuclear transport and non-transport functions. J Biochem 160, 69 (2016).
2. Christie et al. Structural Biology and Regulation of Protein Import into the Nucleus. J Mol Biol 428, 2060 (2016).
3. Panayotis et al. Macromolecular transport in synapse to nucleus communication. Trends Neurosci 38, 108 (2015).
4. Terenzio et al. Compartmentalized signaling in neurons: from cell biology to neuroscience. Neuron 96, 667 (2017).
5. Friedrich et al. Nuclear localization signal and protein context both mediate importin alpha specificity of nuclear import substrates. Mol Cell Biol 26, 8697 (2006).
6. Mackmull et al. Landscape of nuclear transport receptor cargo specificity. Mol Syst Biol 13, 962 (2017).
7. Dohner et al. Importin alpha1 is required for nuclear import of herpes simplex virus proteins and capsid assembly in fibroblasts and neurons. PLoS Pathog 14, e1006823 (2018).
8. Panayotis et al. Importin alpha5 Regulates Anxiety through MeCP2 and Sphingosine Kinase 1. Cell Rep 25, 3169 (2018).
9. Sowa et al. Karyopherin alpha-3 is a key protein in the pathogenesis of spinocerebellar ataxia type 3 controlling the nuclear localization of ataxin-3. Proc Natl Acad Sci USA 115, E2624 (2018).
10. Shmidt et al. Normal brain development in importin-alpha5 deficient-mice. Nat Cell Biol 9, 1337 (2007).
11. Rother et al. Importin alpha7 is essential for zygotic genome activation and early mouse development. PloS One 6, e18310 (2011).
12. Gabriel et al. Differential use of importin-alpha isoforms governs cell tropism and host adaptation of influenza virus. Nat Commun 2, 156 (2011).
13. Decosterd & Woolf. Spared nerve injury: an animal model of persistent peripheral neuropathic pain. Pain 87, 149 (2000).
14. Pertin et al. The spared nerve injury model of neuropathic pain. Methods Mol Biol 851, 205 (2012).
15. Shiers et al. Neuropathic Pain Creates an Enduring Prefrontal Cortex Dysfunction Corrected by the Type II Diabetic Drug Metformin But Not by Gabapentin. J Neurosci 38, 7337 (2018).
16. Coggeshall. Fos, nociception and the dorsal horn. Prog Neurobiol 77, 299 (2005).
17. Santos et al. Fos Protein as a Marker of Neuronal Activity: a Useful Tool in the Study of the Mechanism of Action of Natural Products with Analgesic Activity. Mol Neurobiol 55, 4560 (2018).
18. Lai et al. Dynorphin A activates bradykinin receptors to maintain neuropathic pain. Nat Neurosci 9, 1534 (2006).
19. Arnold et al. Transportin is a major nuclear import receptor for c-Fos: a novel mode of cargo interaction. J Biol Chem 281, 5492 (2006).
20. Aikawa et al. Treatment of arthritis with a selective inhibitor of c-Fos/activator protein-1. Nat Biotechnol 26, 817 (2008).
21. Makino et al. A selective inhibition of c-Fos/activator protein-1 as a potential therapeutic target for intervertebral disc degeneration and associated pain. Sci Rep 7, 16983 (2017).
22. Lamb et al. The Connectivity Map: using gene-expression signatures to connect small molecules, genes, and disease. Science 313, 1929 (2006).
23. Mears et al. Mining the transcriptome for rare disease therapies: a comparison of the efficiencies of two data mining approaches and a targeted cell-based drug screen. NPJ Genom Med 2, 14 (2017).
24. Huang et al. Identifying the pathways required for coping behaviours associated with sustained pain. Nature published online, www(dot)//doi(dot)org/10(dot)1038/s41586 (2018).
25. Ray et al. Comparative transcriptome profiling of the human and mouse dorsal root ganglia: an RNA-seq-based resource for pain and sensory neuroscience research. Pain 159, 1325 (2018).
26. Ben-Yaakov et al. Axonal transcription factors signal retrogradely in lesioned peripheral nerve. The EMBO journal 31, 1350 (2012).
27. Moriyama et al. Targeted disruption of one of the importin alpha family members leads to female functional incompetence in delivery. FEBS J 278, 1561 (2011).
28. Yekkirala et al. Breaking barriers to novel analgesic drug development. Nat Rev Drug Discov 16, 545 (2017).
29. Skolnick & Volkow. Re-energizing the Development of Pain Therapeutics in Light of the Opioid Epidemic. Neuron 92, 294 (2016).

What is claimed is:

1. A method of treating nociceptive or neuropathic pain in a subject in need thereof, the method comprising administering to the subject a therapeutically effective amount of an agent which binds importin α3 or a polynucleotide encoding same and inhibits expression and/or activity of said importin α3, thereby treating the nociceptive or neuropathic pain in the subject.

2. The method of claim 1, wherein said agent binds an importin α3-C-Fos complex, interferes with formation of said importin α3-C-Fos complex or disintegrates said importin α3-C-Fos complex.

3. The method of claim 1, wherein said agent is a small molecule.

4. The method of claim 1, wherein said agent is an RNA silencing agent.

5. The method of claim 1, wherein said agent is an inhibitory peptide.

6. The method of claim 5, wherein said peptide comprises a portion of C-Fos comprising an amino acid sequence of a nuclear localization sequence (NLS) of C-Fos or a portion of C-Jun comprising an amino acid sequence of a nuclear localization sequence (NLS) of C-Jun.

7. A method of treating pain in a subject in need thereof, the method comprising administering to the subject:
(i) a therapeutically effective amount of an agent capable of inhibiting expression and/or activity of a target selected from the targets listed in Table 1; or
(ii) a therapeutically effective amount of an agent capable of enhancing expression and/or activity of a target selected from the targets listed in Table 2.

8. The method of claim 7, wherein said target is Syngap1 or RTL1.

9. The method of claim 7, wherein said agent binds said target or a polynucleotide encoding same.

10. A method of treating pain in a subject in need thereof, the method comprising administering to the subject a therapeutically effective amount of a compound selected from the compounds listed in Table 3, thereby treating pain in the subject.

11. The method of claim 10, wherein said compound is selected from the group consisting of sulmazole, sulfamethizole, ajmaline, pramocaine, prasterone, MK-886, diphenylpyraline, vitexin, ciclacillin, sulfamidine, ceftazidime and profenamine.

12. The method of claim 10, wherein said compound is sulmazole or sulfamethizole.

13. The method of claim 7, wherein said pain is nociceptive or neuropathic pain.

14. The method of claim 1, wherein said pain is acute pain.

15. The method of claim 1, wherein said pain is chronic pain.

16. The method of claim 1, wherein said neuropathic pain is peripheral neuropathic pain.

17. The method of claim 1, wherein said neuropathic pain is central neuropathic pain.

18. The method of claim 1, wherein said pain is a peripheral denervation neuropathic pain.

19. The method of claim 1, wherein said pain is an acute thermal nociceptive pain or acute mechanical nociceptive pain.

20. The method of claim 1, wherein said pain is an acute chemically-induced pain.


SEQUENCE LISTING (22 sequences)

SEQ ID NO: 1 - DNA, 20 bp, Artificial sequence
  Single strand DNA oligonucleotide
ggctgtattcccctccatcg

SEQ ID NO: 2 - DNA, 22 bp, Artificial sequence
  Single strand DNA oligonucleotide
ccagttggtaacaatgccatgt

SEQ ID NO: 3 - DNA, 21 bp, Artificial sequence
  Single strand DNA oligonucleotide
ccagtgatcgaaatccaccaa

SEQ ID NO: 4 - DNA, 22 bp, Artificial sequence
  Single strand DNA oligonucleotide
cgtttgttcagacgttccagat

SEQ ID NO: 5 - DNA, 20 bp, Artificial sequence
  Single strand DNA oligonucleotide
acgtaaacggccacaagttc

SEQ ID NO: 6 - DNA, 20 bp, Artificial sequence
  Single strand DNA oligonucleotide
gtgtacttcgtcgtgctgaa

SEQ ID NO: 7 - DNA, 59 bp, Artificial sequence
  target sequence for importin alpha-3 shRNA
gatccggctttgacaaacattgcatgaagcttgatgcaatgtttgtcaaagcctttttt

SEQ ID NO: 8 - DNA, 59 bp, Artificial Sequence; identical to SEQ ID NO: 7
  simportin a3 hRNA target sequence

SEQ ID NO: 9 - DNA, 23 bp, Artificial sequence
  Single strand DNA oligonucleotide
gggacaaatggattgagaatctg

SEQ ID NO: 10 - DNA, 19 bp, Artificial sequence
  Single strand DNA oligonucleotide
ggcggctgttgtccttgtt

SEQ ID NO: 11 - DNA, 20 bp, Artificial sequence
  Single strand DNA oligonucleotide
acttcctgacggccatcttt

SEQ ID NO: 12 - DNA, 20 bp, Artificial sequence
  Single strand DNA oligonucleotide
gtcgcctgtgctctggtact

SEQ ID NO: 13 - DNA, 18 bp, Artificial sequence
  Single strand DNA oligonucleotide
gcatgcttcgcgtatgca

SEQ ID NO: 14 - DNA, 19 bp, Artificial sequence
  Single strand DNA oligonucleotide
gatggtgcggctgtggata

SEQ ID NO: 15 - DNA, 20 bp, Artificial sequence
  Single strand DNA oligonucleotide
ccgctttcggtatcacaaca

SEQ ID NO: 16 - DNA, 18 bp, Artificial sequence
  Single strand DNA oligonucleotide
cggtctggcgatggaact

SEQ ID NO: 17 - DNA, 64 bp, Artificial sequence
  shFos shRNA target sequence
gatccggcggagacagatcaacttgaagaagcttgttcaagttgatctgtctccgccttttttt

SEQ ID NO: 18 - DNA, 66 bp, Artificial sequence
  shFos 2  shRNA target sequence
gatccgggaccttacctgttcgtgaaacgaagcttggtttcacgaacaggtaaggtcccttttttt

SEQ ID NO: 19 - DNA, 80 bp, Artificial sequence
  shJun shRNA target sequence
gatccggcacatcaccactacaccgacccccacccgaagcttggggtgggggtcggtgtagtggtgatgtgccttttttt

SEQ ID NO: 20 - protein, 25 aa, Artificial Sequence
  NLS AMINO ACID SEQUENCE OF C-FOS
QLSPEEEEKRRIRRERNKMAAAKCR

SEQ ID NO: 21 - protein, 42 aa, Artificial Sequence
  NLS AMINO ACID SEQUENCE OF C-JUN
RIKAERKRMRNRIAASKCRKRKLERIARLEEKVKTLKAQNSE

SEQ ID NO: 22 - DNA, 21 bp, Artificial Sequence
  Single strand DNA oligonucleotide
gccctctcttaccttactgat